(19) United States
(12) Patent Application Publication
(10) Pub. No.: US 2009/0112089 A1
(43) Pub. Date: Apr. 30, 2009

(54) SYSTEM AND METHOD FOR MEASURING BLADDER WALL THICKNESS AND PRESENTING A BLADDER VIRTUAL IMAGE

(76) Inventors: Bill Barnard, Woodinville, WA (US); Stephen Dudycha, Bothell, WA (US); Vikram Chalana, Mill Creek, WA (US); Jongtae Yuk, Redmond, WA (US); Gerald McMorrow, Kirkland, WA (US)

Correspondence Address: BLACK LOWE & GRAHAM, PLLC, 701 FIFTH AVENUE, SUITE 4800, SEATTLE, WA, 98104, US

(21) Appl. No.: 11/925,850

(22) Filed: Oct. 27, 2007

(51) Int. Cl. A61B 8/00

(52) U.S. Cl. 600/443; 600/449

(57) ABSTRACT

An ultrasound transceiver scans a bladder in a three dimensional array to measure the thickness and surface area of the bladder to determine bladder mass. The bladder wall thickness and masses may be determined for anterior, posterior, and lateral locations of the bladder.

### FIELD OF THE INVENTION

This invention relates generally to using ultrasound in diagnosing bladder condition or dysfunction.

### BACKGROUND OF THE INVENTION

The following applications are incorporated by reference as if fully set forth herein: U.S. application Ser. No. 10/704,996 filed Nov. 10, 2003; Ser. No. 11/061,867 filed Feb. 17, 2005 and Ser. No. 11/295,043 filed Dec. 6, 2005.

A variety of techniques have been used to evaluate bladder dysfunction. Such techniques typically attempt to determine the size of the bladder or bladder volume, meaning the amount of urine in the bladder. As one example, U.S. Pat. No. 6,110,111 to Barnard discloses a system for assessing bladder distension by using ultrasound to compare the bladder surface area with the surface area of a sphere. According to Barnard, the closer the bladder is to a spherical shape, the greater the pressure within the bladder.

Bladder mass measurements can also be used to diagnose several different clinical conditions. Bladder wall thickness and bladder mass can be used to indicate bladder outlet obstruction and bladder distension. An outlet obstruction will cause a higher pressure in the urine, against which the bladder muscle must contract. That higher pressure causes the muscle to exert more force, resulting in hypertrophy of the bladder muscle. Symptoms of bladder muscle hypertrophy include increased wall thickness and increased mass. The use of bladder wall thickness as an indicator of detrusor hypertrophy has been noted for many years (see Matthews P N, Quayle J B, Joseph A E A, Williams J E, Wilkinson K W, Riddle P R, The use of ultrasound in the investigation of prostatism, British Journal of Urology, 54:536-538, 1982; and Cascione C J, Bartone F F, Hussain M B, Transabdominal ultrasound versus excretory urography in preoperative evaluation of patients with prostatism, Journal of Urology, 137:883-885, 1987). Converting bladder wall thickness to bladder wall volume (or bladder mass by multiplying bladder wall volume by the specific gravity of bladder tissue) yields a single number, which is independent of bladder volume. While the bladder wall thins as volume increases, the total bladder wall volume (or bladder mass) remains unchanged.

Another key parameter of bladder functionality is bladder distension. As the bladder volume and bladder pressure increases, the bladder walls stretch and thin. Two prominent maladies associated with bladder distension are incontinence and hyperdistension.

Incontinent episodes frequently occur if the bladder sphincter muscles are unable to retain urine as bladder pressure and bladder distension increases. In many individuals, this incontinent point occurs at a consistent volume. Consequently, if this volume is known and if the bladder volume can be measured over time, then incontinent events can be prevented. Furthermore, research has shown that it is possible to increase both the bladder capacity and the bladder volume incontinent point through a variety of methods. This technique has been used effectively on enuretic patients.

Hyperdistension refers to the case in which the bladder is allowed to fill to such an extreme that excessive bladder pressure builds which can cause potential renal damage, renal failure and even patient death from autonomic dysreflexia if the patient has spinal cord damage. As with incontinence, hyperdistension has been successfully prevented using non-invasive bladder volume measuring.

At small bladder volumes, bladder response is quite constant across humanity. Normal adult humans typically have no trouble voiding and leaving less than 50 ml of urine. Thus, it has been relatively easy to establish post-void-residual (PVR) volumes that are normal and PVR volumes that are potential medical problems. At low bladder volumes, bladder distension information is not as useful. However, normal humans have widely variant bladder capacities. Thus, it is more difficult to establish a volume threshold at which over-distension occurs or when incontinence occurs. As the bladder fills, quantization of bladder distension becomes more useful. This is especially true since it is thought that a bladder distension metric would better indicate hyperdistension and bladder capacity.

Current methods to measure bladder wall thickness rely on one-dimensional (A-mode) and two-dimensional (B-mode) ultrasound and are greatly susceptible to operator error, time consuming, and inaccurate. The operator using one or two-dimensional ultrasound has to repeatedly reposition the ultrasound probe until a bladder wall image is sufficiently visible, usually the more anterior portion of the bladder. Furthermore, the limitations of one and two-dimensional ultrasound require inaccurate spherical model assumptions for the bladder. Presumably for these and other reasons, the industry has concluded that measuring bladder wall thickness is an unreliable or ineffective means to quantize bladder distension. See, e.g., Barnard, U.S. Pat. No. 6,110,111 at column 1, lines 50-59.

Thus, there is a need for a system to accurately measure bladder wall thickness for use in evaluating bladder distension.

A variety of ultrasound methods may be used to evaluate a bladder dysfunction. In general, such methods estimate a bladder volume containing an amount of urine. For example, U.S. Pat. No. 6,110,111 to Barnard discloses an ultrasound system for estimating bladder pressure by comparing the estimated bladder surface area with the surface area of a comparable sphere. According to Barnard, as the bladder surface area approaches the surface area of the comparable sphere, a greater pressure within the bladder is inferred.

Other bladder measurements are possible using ultrasound methods, and are similarly useful in the diagnosis of several different bladder conditions. For example, a bladder wall thickness and bladder mass may be estimated using ultrasound, and may be used to indicate a bladder outlet obstruction and/or a bladder distension. In general, a bladder outlet obstruction results in an elevated internal pressure in the bladder that must be overcome by the surrounding muscle as the bladder contracts during urination. Accordingly, an undesired hypertrophy of the bladder muscle often results. Symptoms of bladder muscle hypertrophy generally include increased bladder wall thickness and increased bladder wall mass. See, for example, P. N. Matthews, J. B. Quayle, A. E. A. Joseph, J. E. Williams, K. W. Wilkinson and P. R. Riddle; “The Use of Ultrasound in the Investigation of Prostatism”, British Journal of Urology, 54:536-538, 1982; and C. J. Cascione, F. F. Bartone and M. B. Hussain; “Transabdominal Ultrasound Versus Excretory Urography in Preoperative Evaluation of Patients with Prostatism”, Journal of Urology, 137:883-885, 1987). Using an estimated bladder wall thickness to infer a bladder wall volume, or, alternately, a bladder wall mass (obtained by multiplying the estimated bladder wall volume by a specific gravity of the bladder tissue) yields a value that is generally independent of the bladder volume. While the bladder wall thins as the volume increases, the total bladder wall volume (or the bladder wall mass) remains generally unchanged.

Another indicator of the bladder condition is bladder distension. As the bladder volume increases in response to increased internal bladder pressure, the bladder walls elongate and decrease in thickness, resulting in the distention. Bladder distention is generally associated with numerous bladder ailments, including incontinence and hyperdistension. Incontinence occurs when sphincter muscles associated with the bladder are unable to retain urine within the bladder as the bladder pressure and bladder distension increases. In many individuals, incontinence occurs when the bladder volume achieves a consistent maximum volume in the individual. Consequently, if the maximum volume is known, and if the bladder volume can be measured while the volume is approaching the maximum value, incontinence may be prevented. When hyperdistension occurs, the bladder fills with an excessive amount urine and generates an internal bladder pressure that may cause serious adverse effects, including renal damage, renal failure, or even death of the patient from autonomic dysreflexia if the patient has spinal cord damage.

It is further observed that normal bladder response is relatively constant at small bladder volumes in typical adult humans. Accordingly, normal healthy adults encounter little physical difficulty voiding, and typically leave less than about 50 milliliters (ml) of urine in the bladder. Thus at the present time, it is relatively easy to distinguish a normal post-void-residual (PVR) volume from an abnormal PVR volume that may be indicative of a potential medical problem. At low bladder volumes, bladder distension information is not typically useful since normal humans have widely varying bladder capacities. Thus, it is more difficult to establish a volume threshold at which over-distension occurs or when incontinence occurs for a selected individual. Consequently, as the bladder fills, measurement of bladder distension becomes more useful as an indicator of hyperdistension and bladder capacity in an individual.

Current ultrasound methods measure bladder wall thicknesses using one-dimensional (A-mode) and two-dimensional (B-mode) ultrasound modes. Unfortunately, the application of these current methods to determine bladder wall thickness are susceptible to operator error, are time consuming, and generally lead to inaccurate estimations of the bladder wall thickness. For example, in one known ultrasound method, an operator applies an ultrasound probe to an external portion of the patient and projects ultrasound energy into the patient to image a bladder region. Since the operator must repeatedly reposition the ultrasound probe until a bladder wall image is sufficiently visible, inaccuracies may be introduced into the ultrasound data. Consequently, current ultrasound methods to determine bladder wall thickness is an unreliable or ineffective means to measure bladder distension.

Thus, there is a need for an ultrasound method and system that permits a bladder wall thickness to be accurately measured.

Benign prostate hyperplasia (BPH) and other disorders can cause mechanical bladder outlet obstruction (BOO). A marker for predicting BOO is determining the weight of the bladder wall. Using probing ultrasound, an ultrasound estimated bladder wall weight (UEBW) might be obtained in a non-invasive way. Existing methods for acquiring UEBW assumes that the bladder is spherically shaped and that the thickness of the bladder wall is relatively constant in near empty to nearly full bladders. Moreover, the existing 2D methods are manually based, utilizing leading edge-to-leading edge of opposing bladder walls laboriously executed upon a series of two-dimensional images, and are fraught with analytical inaccuracies (H. Miyashita, M. Kojima, and T. Miki, “Ultrasonic measurement of bladder weight as a possible predictor of acute urinary retention in men with lower urinary tract symptoms suggestive of benign prostate hyperplasia”, Ultrasound in Medicine and Biology 2002, 28(8): 985-990; M. Oelke, K. Hofner, B. Wiese, V. Gruneweld, and U. Jonas, “Increase in detrusor wall thickness indicates bladder outlet obstruction in men,” World J. of Urology, 2002, 19(6), 443-452; L. Muller, T. Bergstrom, M. Hellstrom, E. Svensson, and B. Jacobson, “Standardized ultrasound method for assessing detrusor muscle thickness in children,” J. Urol., 200, 164: 134-138; and Naya, M. Kojima, H. Honjyo, A. Ochiai, O. Ukimura, and H. Watanabe, “Intraobserver and interobserver variance in the measurement of ultrasound-estimated bladder weight,” Ultrasound in Med. & Biol., 1999, 24(5): 771-773).

There is a need to accurately and non-invasively determine bladder wall weight by accurately measuring bladder wall volume to avoid incurring the errors invoked by the fixed bladder shape assumptions and those generated by the manual image processing methods of 2D acquired ultrasound images.

### SUMMARY OF THE INVENTION

The present invention incorporates a three-dimensional ultrasound device to scan a patient's bladder. Data collected in the ultrasound scan are presented in an array of 2D scanplanes and in a substantially bas-relief 2D presentation of bladder hemispheres showing the bladder wall. The collected data is analyzed to calculate bladder thickness and mass. Bladder mass information is then used to assess bladder dysfunction.

In accordance with the preferred embodiment of the invention, a microprocessor-based ultrasound apparatus, placed on the exterior of a patient, scans the bladder of the patient in multiple planes with ultrasound pulses, receives reflected echoes along each plane, transforms the echoes to analog signals, converts the analog signals to digital signals, and downloads the digital signals to a computer system.

Although a variety of scanning and analysis methods may be suitable in accordance with this invention, in a preferred embodiment the computer system performs scan conversion on the downloaded digital signals to obtain a three-dimensional, conically shaped image of a portion of the bladder from mathematical analysis of echoes reflecting from the inner (submucosal) and outer (subserosal) surfaces of the bladder wall. The conical image is obtained via three-dimensional C-mode ultrasound pulse echoing using radio frequency (RF) ultrasound (approximately 2-10 MHz) to obtain a 3D array of 2D scanplanes, such that the scanplanes may be a regularly spaced array, an irregular spaced array, or a combination of a regularly spaced array and irregularly spaced array of 2D scanplanes. The 2D scanplanes, in turn, are formed by an array of one-dimensional scanlines (ultrasound A-lines), such that the scanlines may be regularly spaced, irregularly spaced, or a combination of regularly spaced and irregularly spaced scanlines. The 3D array of 2D scanplanes results in a solid angle scan cone.

Alternatively, a solid angle scan cone is obtained by 3D data sets acquired from a three-dimensional ultrasound device configured to scan a bladder in a 3D scan cone of 3D distributed scanlines. The 3D scan cone is not a 3D array of 2D scanplanes, but instead is a solid angle scan cone formed by a plurality of internal and peripheral one-dimensional scanlines. The scanlines are ultrasound A-lines that are not necessarily confined within a scanplane, but would otherwise occupy the inter-scanplane spaces that are in the 3D array of 2D scanplanes.

The solid angle scan cones, either as a 3D array of 2D scanplanes, or as a 3D scan cone of 3D distributed scanlines, provides the basis to locate bladder wall regions or surface patches of the inner and outer surfaces of the bladder wall. The location of each surface patch is determined using fractal analytical methods and the distance or thickness between the inner and outer surface patches is measured. The bladder wall mass is calculated as a product of the surface area of the bladder, the bladder wall thickness, and the specific gravity of the bladder wall. The entire bladder wall or various regions, including anterior, posterior, and lateral portions of the bladder, may be measured for thickness and mass.

An alternate embodiment of the invention configures the downloaded digital signals to be compatible with a remote microprocessor apparatus controlled by an Internet web-based system. The Internet web-based system has multiple programs that collect, analyze, and store organ thickness and organ mass determinations. The alternate embodiment thus provides an ability to measure the rate at which internal organs undergo hypertrophy over time. Furthermore, the programs include instructions to permit disease tracking, disease progression, and provide educational instructions to patients.

Another embodiment of the invention presents the bladder, obtained from the 3D array of 2D scanplanes or the 3D scan cone of 3D distributed scanlines, in a substantially 2D bas relief image. The effect is to have the three-dimensional ultrasound device function as a virtual cystoscope. The bas-relief image presents the bladder in cross sectional hemispheres, where the bladder, the bladder wall thickness, and structures in the bladder and bladder wall are visible as a virtual 3D-like image. The virtual bas-relief image is obtained by remote, non-intrusive ultrasound scans processed to present a similar image that would otherwise be obtained by an intrusive, visible light cystoscope.

Systems and methods for ultrasound imaging an abdominal region in a patient to detect and measure underlying organ structures, and in particular, to image a bladder to determine the thickness, volume and mass of the bladder detrussor are disclosed. In an aspect of the invention, echogenic data is obtained by scanning the abdominal region to obtain a three-dimensional scancone assembly comprised of two-dimensional scanplanes, or an array of three-dimensional distributed scanlines. Selected two-dimensional and one-dimensional algorithms are then applied to the echogenic data to measure the bladder wall thickness and surface area.

The pixel location of initial wall loci are determined in two-dimensional scanplanes via B-mode echo signal processing algorithms applied to scanlines crossing the organ wall. The pixel location of the initial wall loci serve as an initial approximation of wall location from which more exacting algorithms are applied to either reconfirm the initially selected wall loci, or more likely, to select other loci positions. The reconfirmed or newly selected loci positions are achieved by the application of higher resolving, echo signal processing algorithms to define final wall loci pixel locations. Thereafter, verification of the final wall loci pixel locations are established by cost function analysis using neighboring final pixel locations of scanlines within the same scanplane.

The final wall pixel loci as determined include the organ outer-wall and the organ inner-wall pixel locations. The distance separating the organ outer-wall and inner-wall final pixel loci determines the thickness of the organ wall. B-mode algorithms applied to the final outer-wall loci pixel locations, as determined by the A-mode algorithms, determine the outer boundary of the organ wall within a given scanplane. Surface area of the inner-wall boundary is determined by analysis of the scanplane arrays within the scancone. Organ wall volume is calculated as a product of organ wall surface area and thickness. Organ wall mass is determined as a product of organ wall volume and density. When the organ is a bladder, the bladder wall thickness and wall mass is calculated to provide information to assess bladder dysfunction.

The collection of two-dimensional and one-dimensional algorithms includes ultrasound B-mode based segmentation and specialized snake algorithms to determine the surface area of the organ wall and to provide an initial front wall location. The initial front wall location determined by the B-mode algorithms is sufficiently precise to be further processed by the one-dimensional algorithms. The one-dimensional algorithms are unique sequences of A-mode based algorithms applied to the echogenic ultrasound scanlines to further improve the accuracy and precision of wall location loci as initially determined by the B-mode algorithms.

In accordance with the preferred embodiment of the invention, a microprocessor-based ultrasound apparatus, placed on the exterior of a patient, scans the bladder of the patient in multiple planes with ultrasound pulses, receives reflected echoes along each plane, transforms the echoes to analog signals, converts the analog signals to digital signals, and downloads the digital signals to a computer system.

Although a variety of scanning and analysis methods may be suitable in accordance with this invention, in a preferred embodiment the computer system performs scan conversion on the downloaded digital signals to obtain a three-dimensional, conically shaped image of a portion of the bladder from mathematical analysis of echoes reflecting from the inner (submucosal) and outer (subserosal) surfaces of the bladder wall. The conical image is obtained via ultrasound pulse echoing using radio frequency (RF) ultrasound (approximately 2-10 MHz) to obtain a three-dimensional array of two-dimensional scanplanes, such that the scanplanes may be a regularly spaced array, an irregular spaced array, or a combination of a regularly spaced array and irregularly spaced array of two-dimensional scanplanes. The two-dimensional scanplanes, in turn are formed by an array of one-dimensional scanlines (ultrasound A-lines), such that the scanlines may be regularly spaced, irregularly spaced, or a combination of regularly spaced and irregularly spaced scanlines. The three-dimensional array of two-dimensional scanplanes results in a solid angle scan cone.

Alternatively, a solid angle scan cone is obtained by three-dimensional data sets acquired from a three-dimensional ultrasound device configured to scan a bladder in a three-dimensional scan cone of three-dimensional distributed scanlines. The three-dimensional scan cone is not a three-dimensional array of two-dimensional scanplanes, but instead is a solid angle scan cone formed by a plurality of internal and peripheral one-dimensional scanlines. The scanlines are ultrasound A-lines that are not necessarily confined within a scanplane, but would otherwise occupy the inter-scanplane spaces that are in the three-dimensional array of two-dimensional scanplanes.

The solid angle scan cones, either as a three-dimensional array of two-dimensional scanplanes, or as a three-dimensional scan cone of three-dimensional distributed scanlines, provides the basis to locate bladder wall regions or surface patches of the inner and outer surfaces of the bladder wall. The location of each surface patch is determined and the distance or thickness between the inner and outer surface patches is measured. The bladder wall mass is calculated as a product of the surface area of the bladder, the bladder wall thickness, and the specific gravity of the bladder wall. The entire bladder wall or various regions, including anterior, posterior, and lateral portions of the bladder, may be measured for thickness and mass. Preferred embodiments of the programs to analyze scanline or scanplane data to determine bladder thickness and mass employ algorithms.

An alternate embodiment of the invention configures the downloaded digital signals to be compatible with a remote microprocessor apparatus controlled by an Internet web-based system. The Internet web-based system has multiple programs that collect, analyze, and store organ thickness and organ mass determinations. The alternate embodiment can measure the rate at which internal organs undergo hypertrophy over time. The programs can include instructions to permit disease tracking, disease progression, and provide educational instructions to patients.

A method and system to acquire an ultrasound-estimated organ wall mass or weight from three dimensional ultrasound echo information is disclosed.

### BRIEF DESCRIPTION OF THE DRAWINGS

The preferred and alternative embodiments of the present invention are described in detail below with reference to the following drawings.

FIG. 1 is a microprocessor-controlled transceiver;

FIG. 2 is a representation of scanlines sharing a common rotational angle to form a plane;

FIG. 3 is a side view representation of a collection of scanplanes that are separated by approximately 7.5 degrees from each other;

FIG. 4 is a top view representation of a collection of planes, each rotated 7.5 degrees from each other;

FIG. 5 is a graphical representation of a plurality of 3D distributed scanlines emanating from the transceiver forming a scan cone;

FIG. 6 is an algorithm for measuring bladder thickness and mass;

FIG. 7 is a representation of four surface patch elements, each constructed from the sixteen neighboring points that surround the patch;

FIG. 8 is a representation of three scanlines passing through the subserosal and submucosal wall locations of the bladder; and

FIG. 9 depicts a substantially bas-relief 2D presentation volume rendering of the left and right half bladder hemisphere views of a bladder.

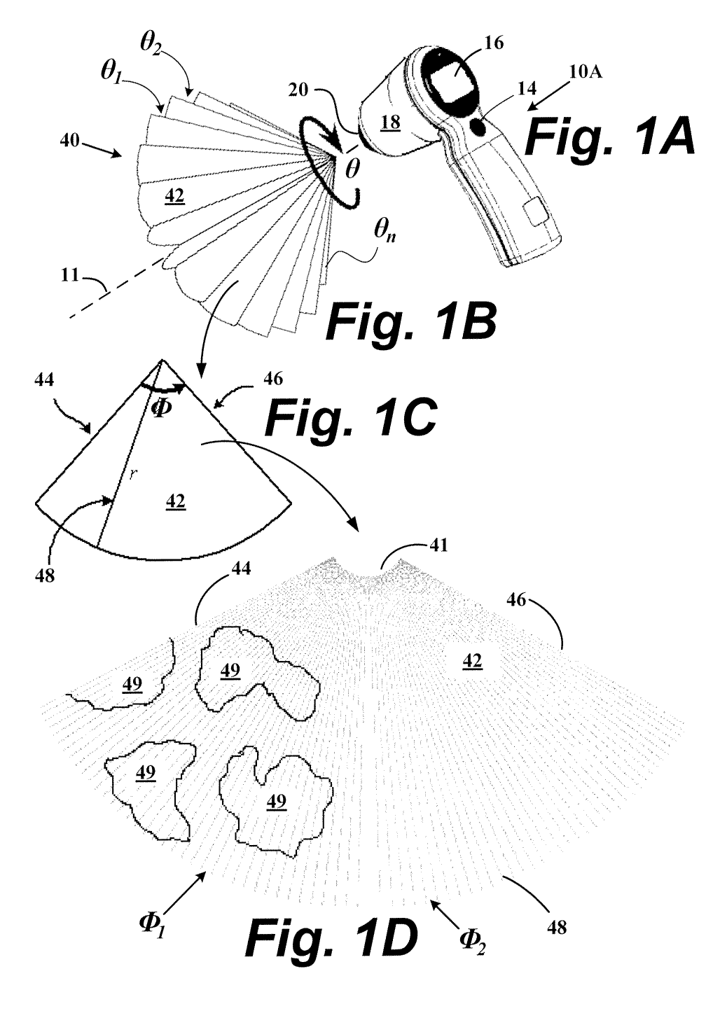

FIG. 1 is a side elevational view of an ultrasound transceiver according to an embodiment of the invention;

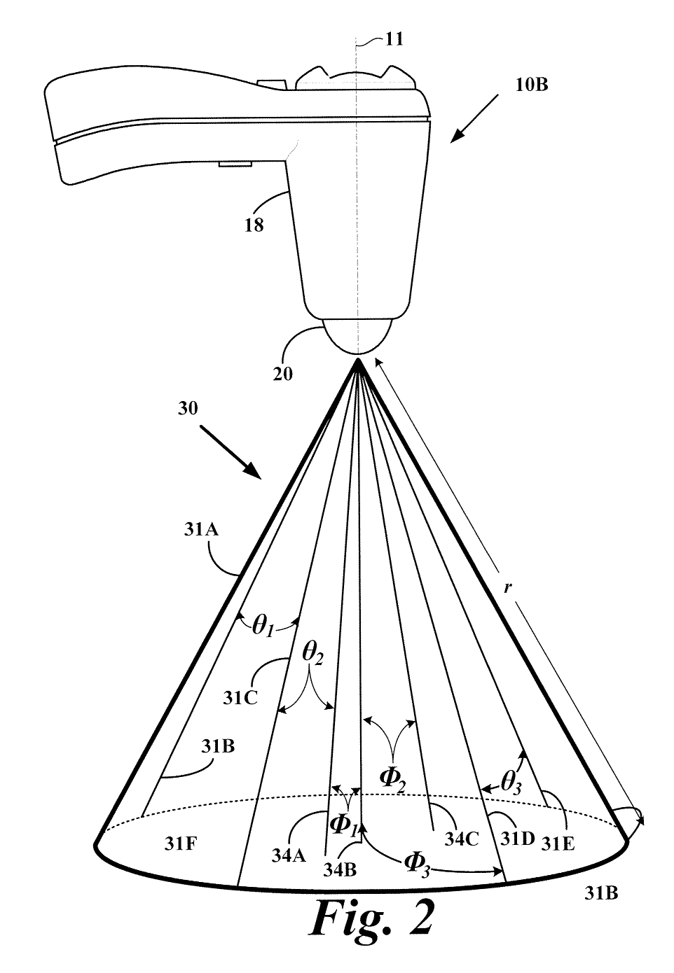

FIG. 2 is an isometric view of an ultrasound scancone that projects outwardly from the transceiver of FIG. 1;

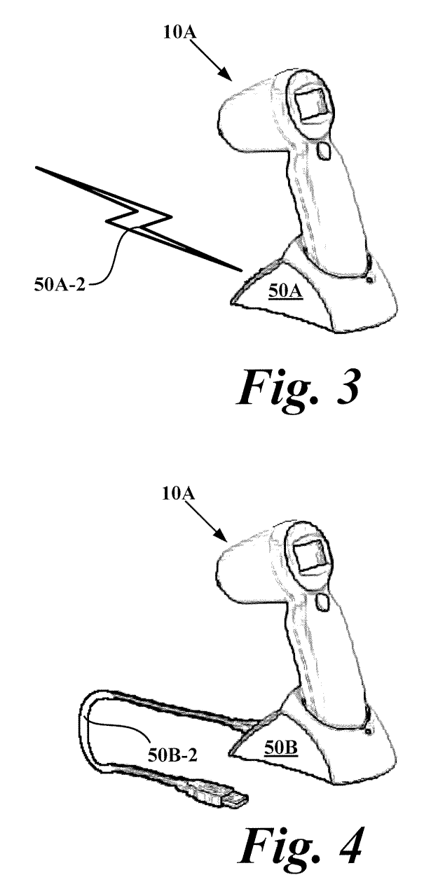

FIG. 3A is a plan view of an ultrasound scanplane representation of an ultrasound scancone that projects outwardly from the transceiver of FIG. 1;

FIG. 3B is an isometric view of an ultrasound scancone that projects outwardly from the transceiver of FIG. 1;

FIG. 3C is a scancone that is generated by the transceiver of FIG. 1;

FIG. 3D is a plan view of the scancone of FIG. 3C;

FIG. 3E is side-elevational view of the scanplane of FIG. 3C and FIG. 3D;

FIG. 4A is an isometric view of the transceiver of FIG. 1 applied to an abdominal region of a patient;

FIG. 4B is a perspective view of the transceiver of FIG. 1 positioned in a communication cradle according to another embodiment of the invention;

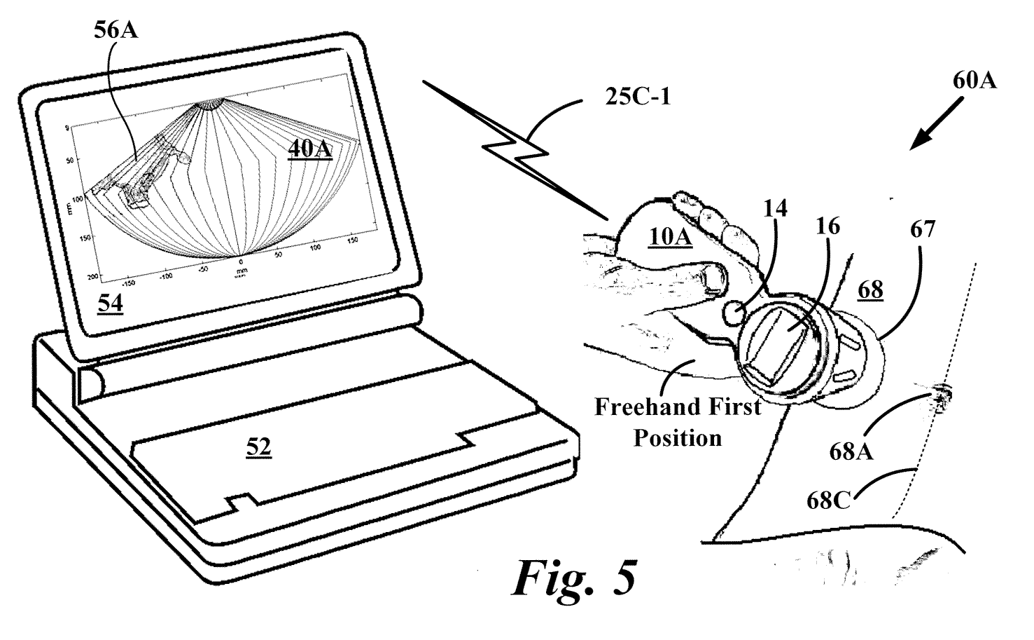

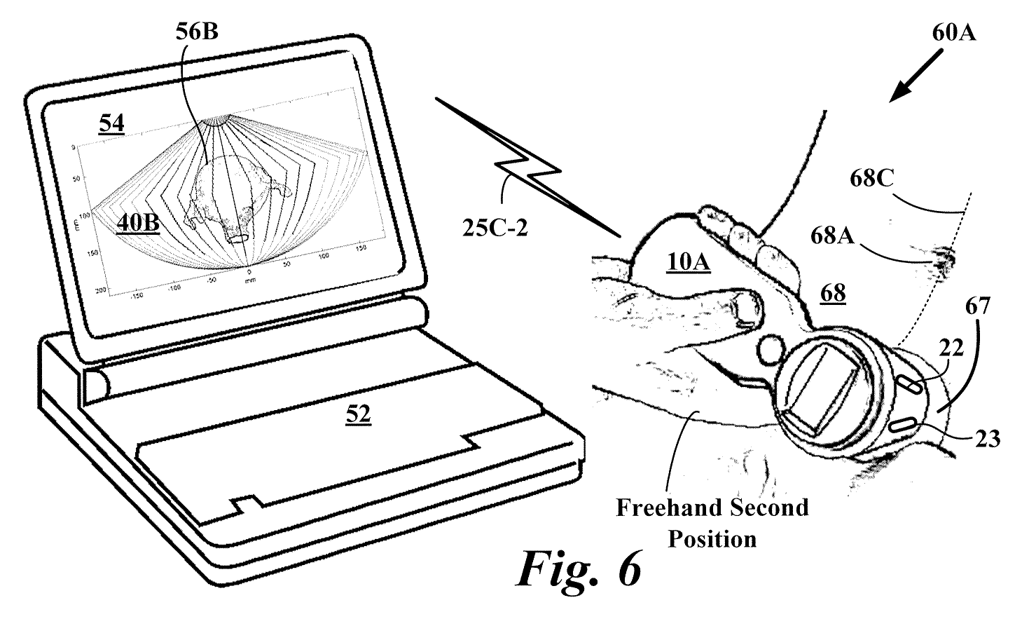

FIG. 5 is a partially-schematic view of an imaging system according to another embodiment of the invention;

FIG. 6 is a partially-schematic view of a networked imaging system according to still another embodiment of the invention;

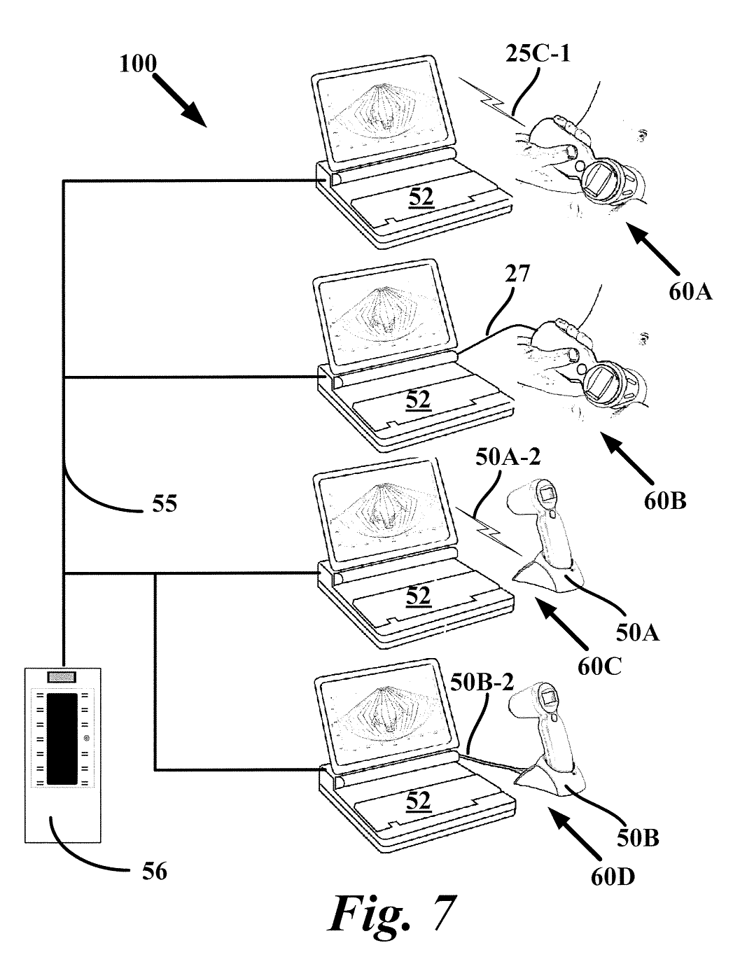

FIG. 7 is a cross sectional view of a selected anatomical portion that will be used to further describe the various embodiments of the present invention;

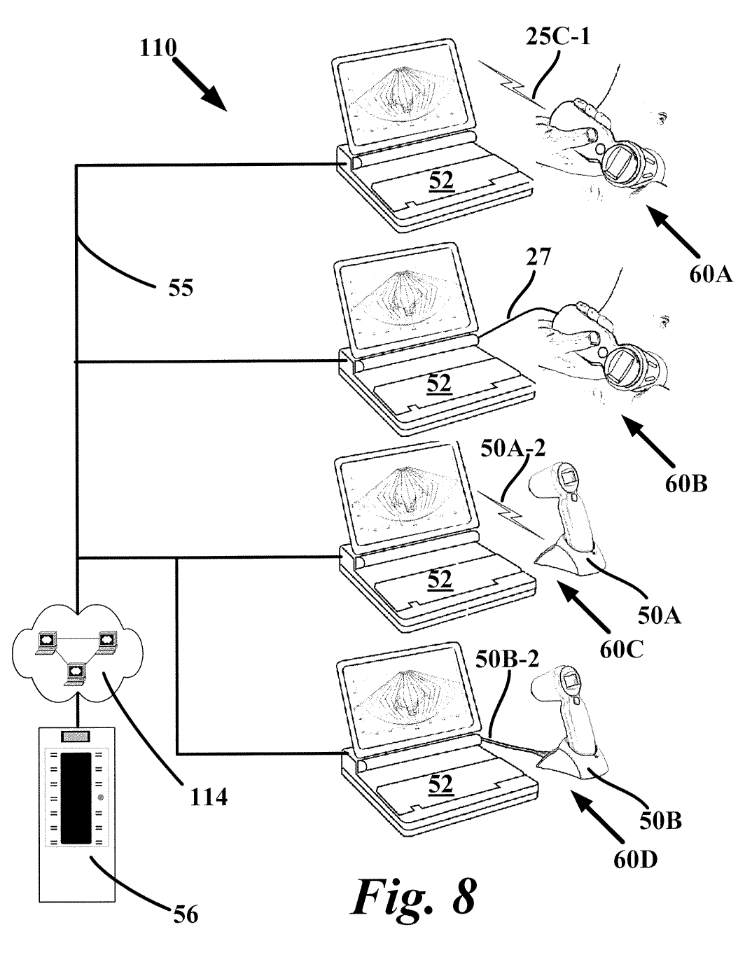

FIG. 8 is a cross sectional view of the anatomical region of FIG. 7 as the region is imaged by the transceiver of FIG. 1;

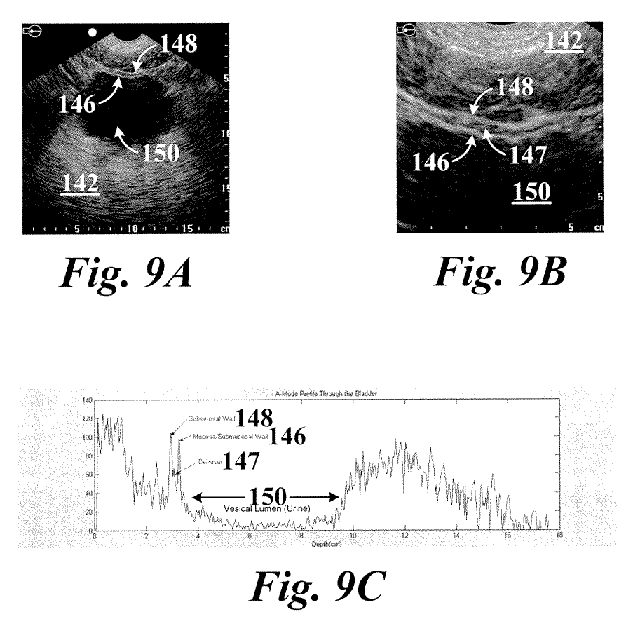

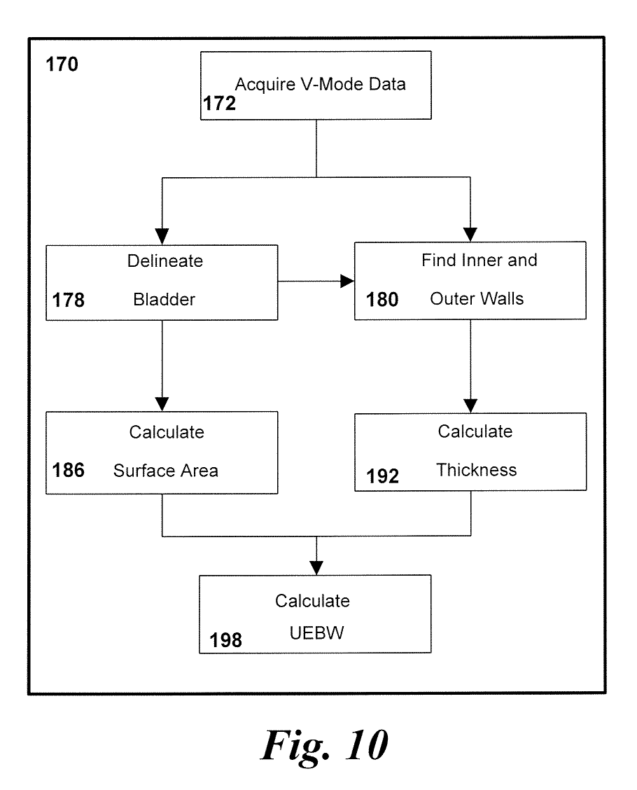

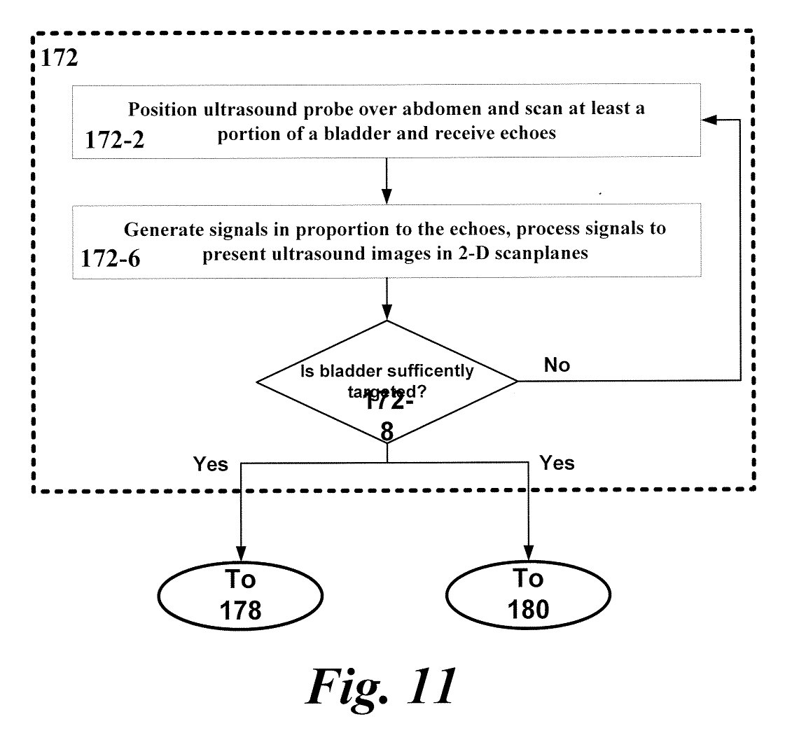

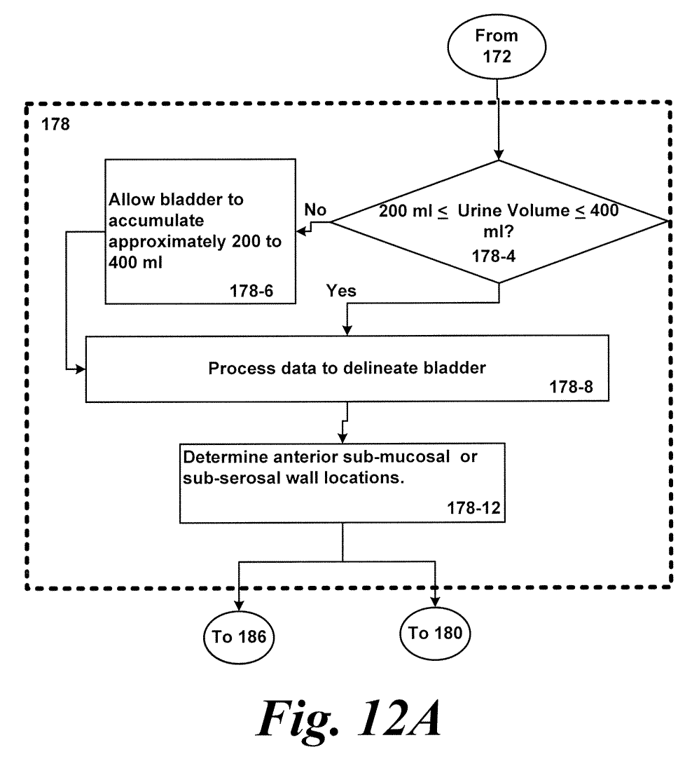

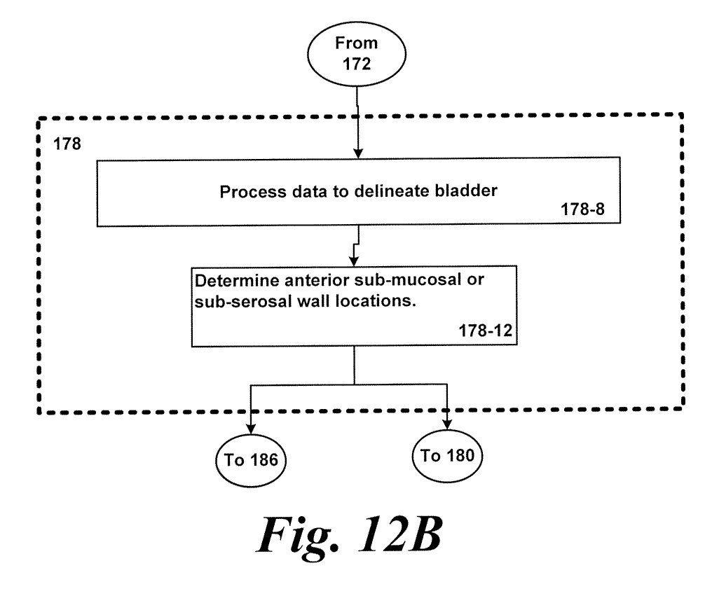

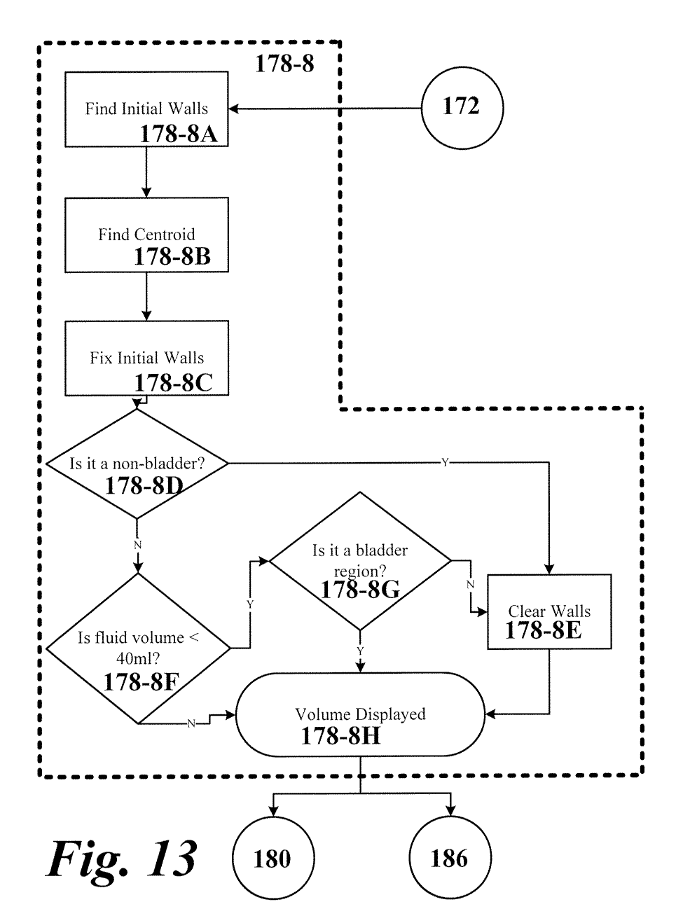

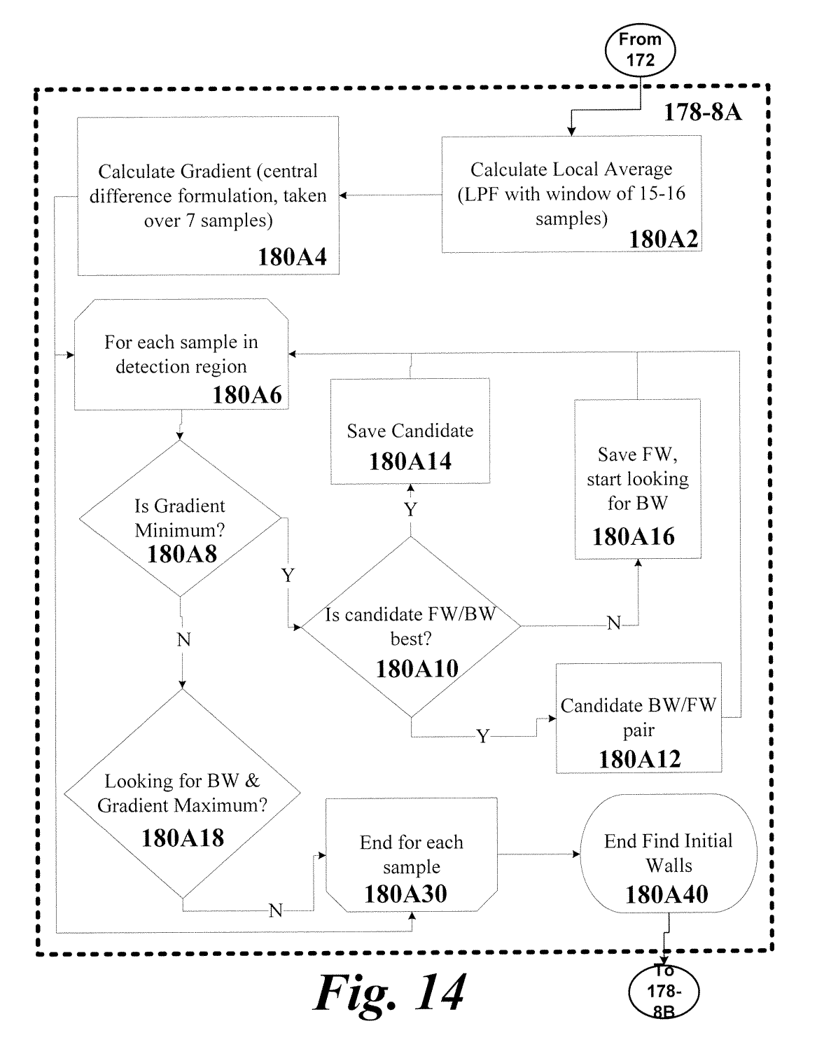

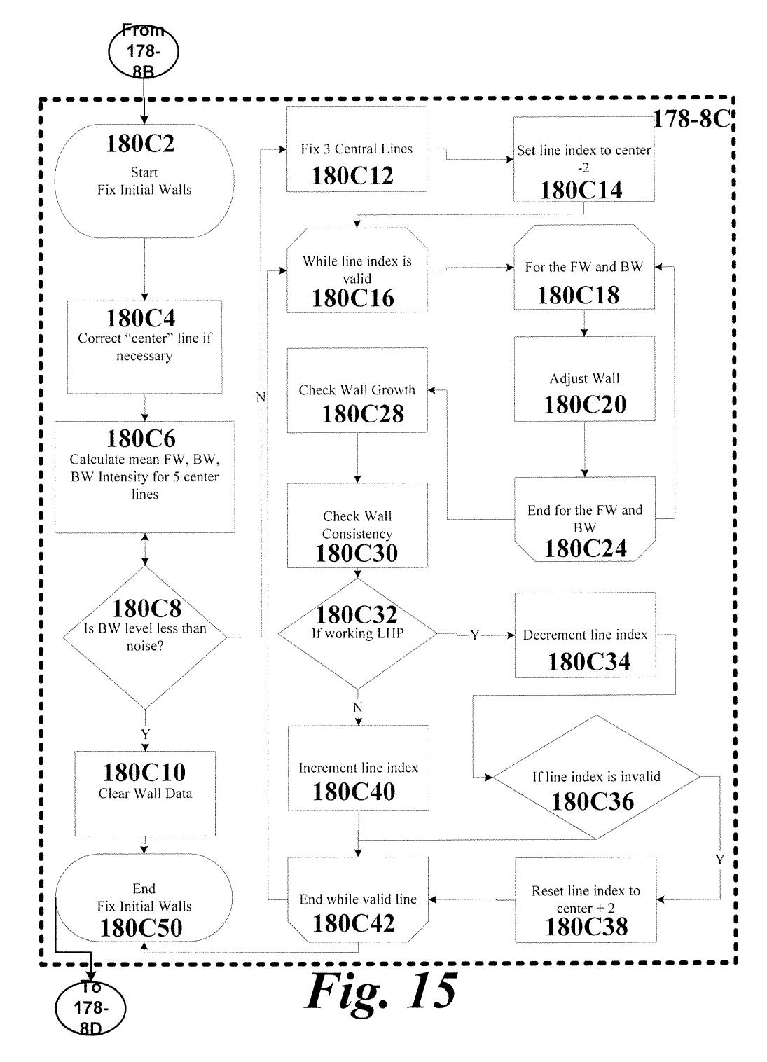

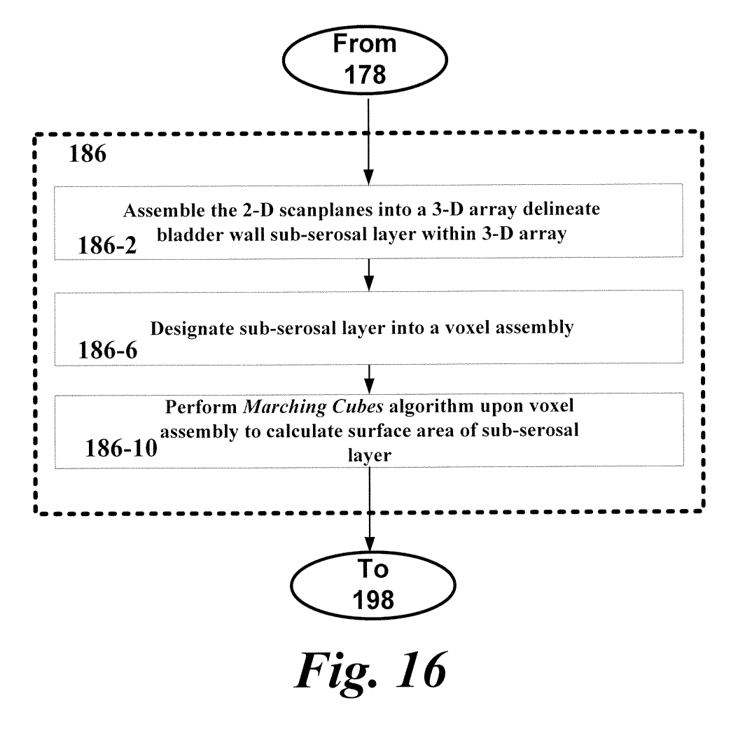

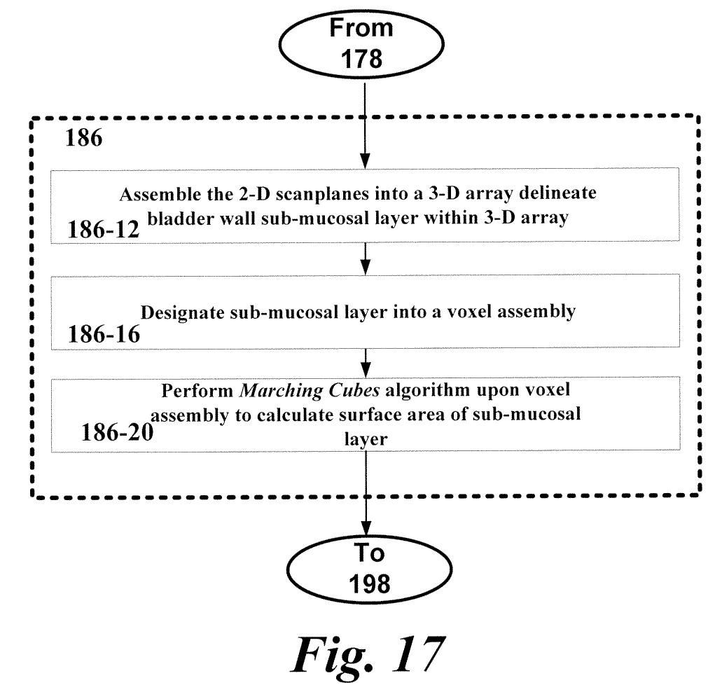

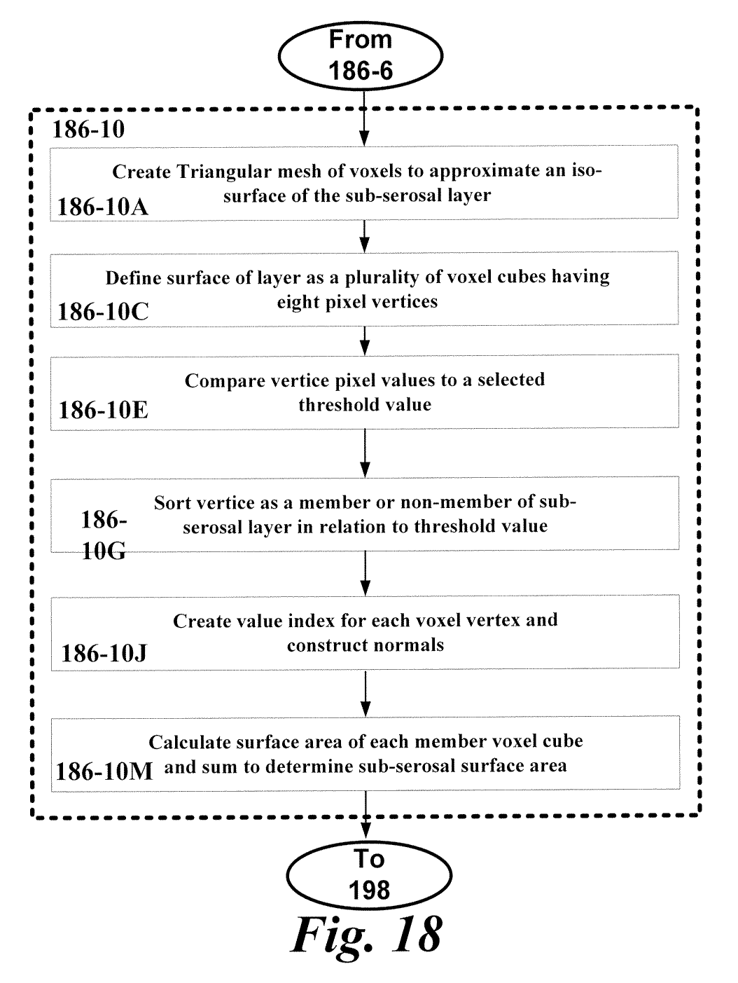

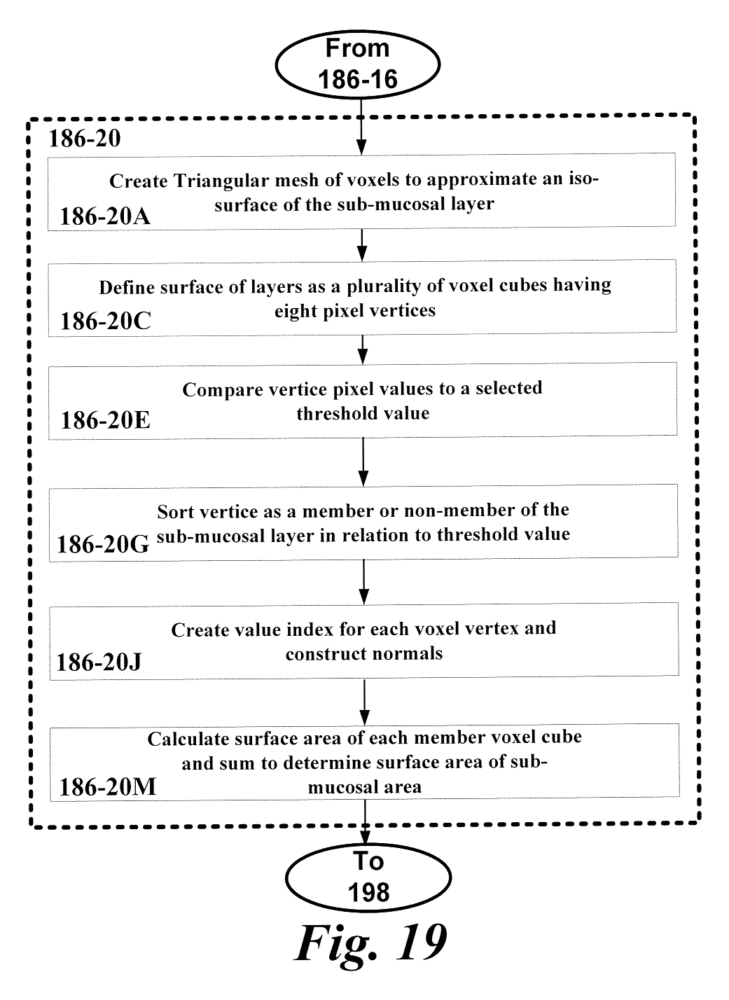

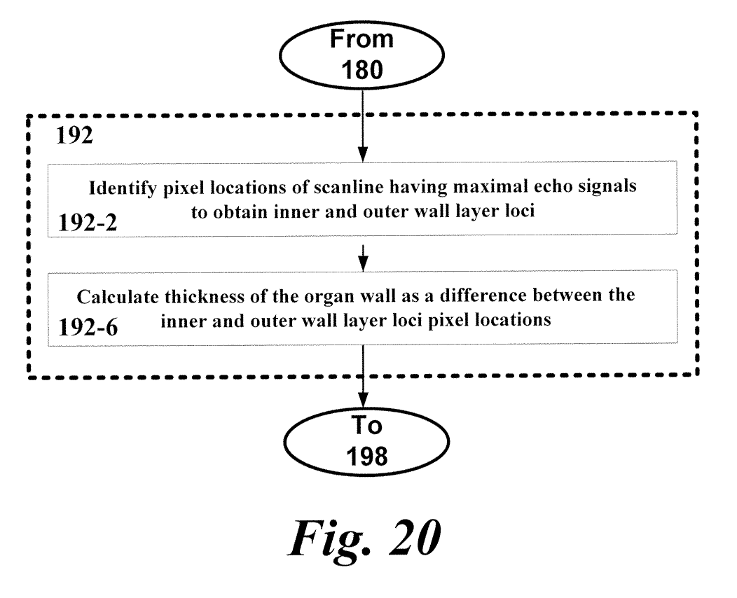

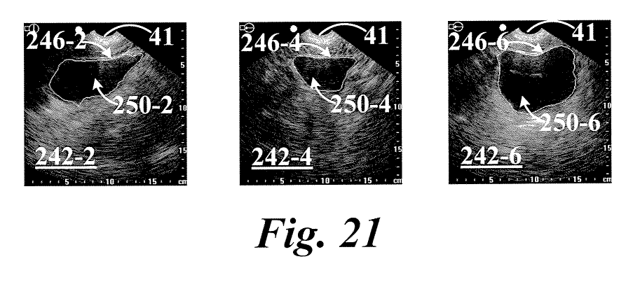

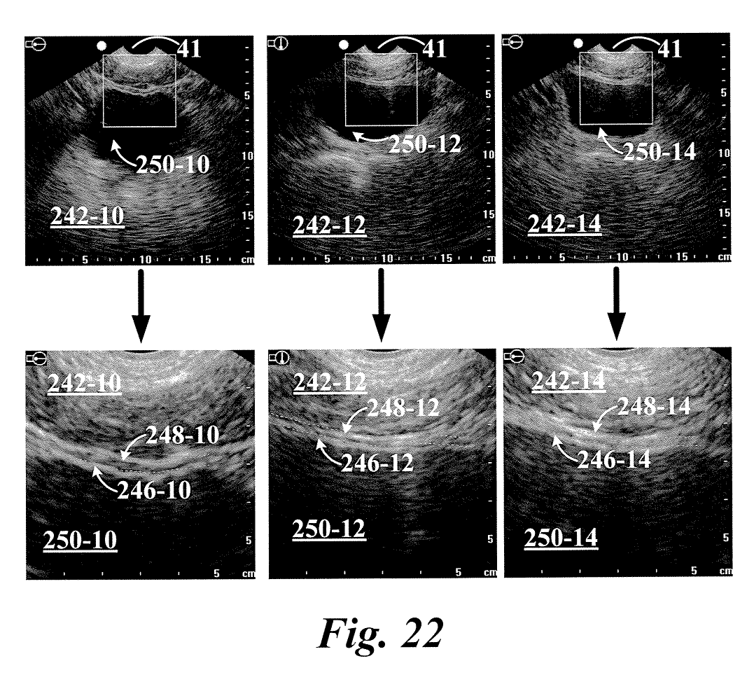

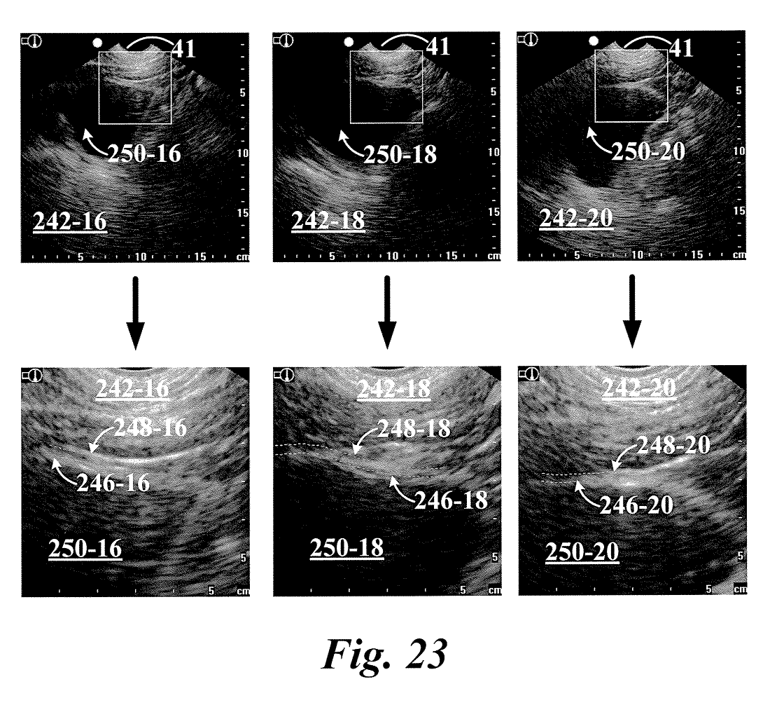

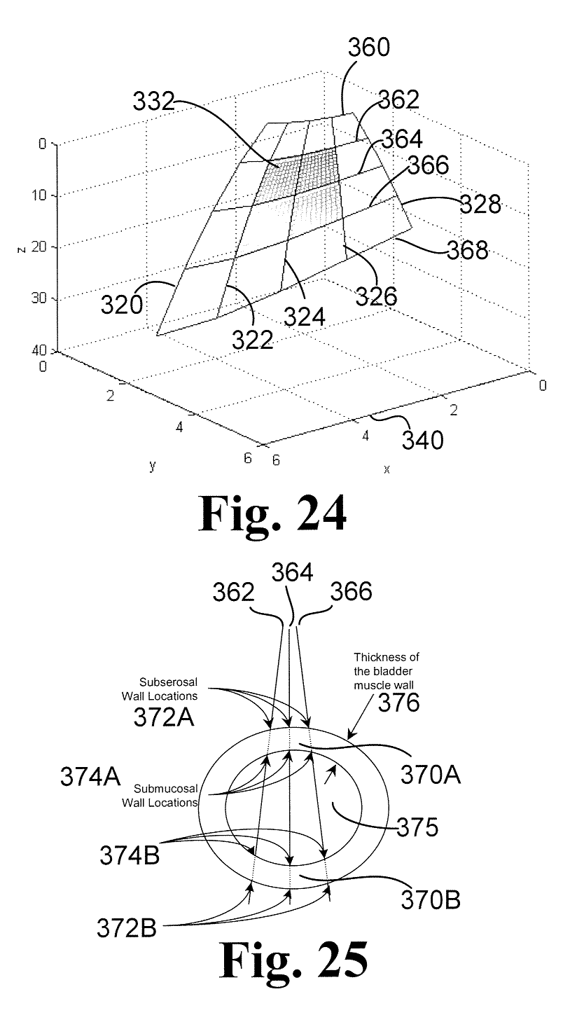

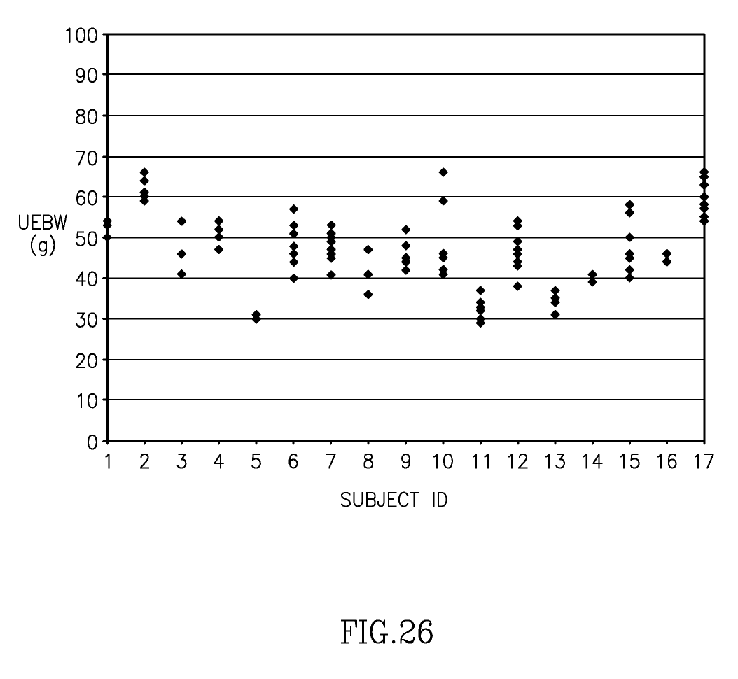

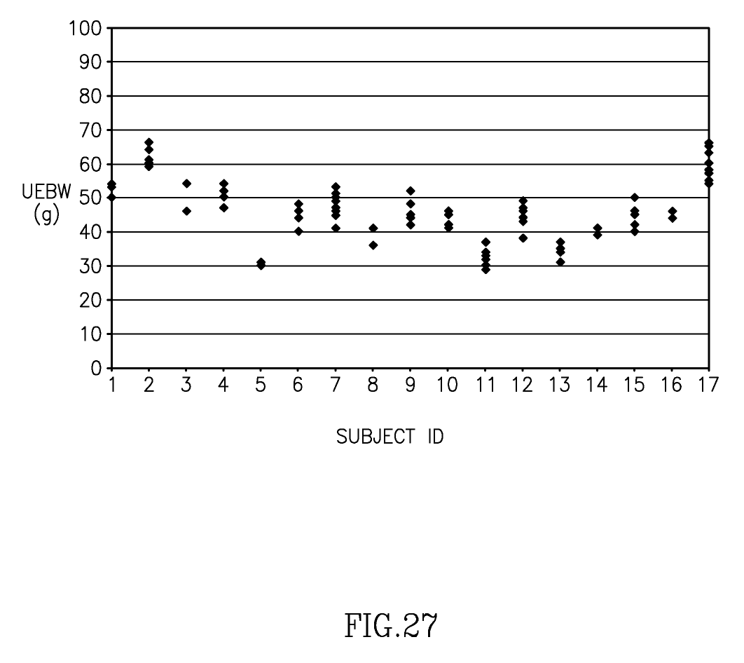

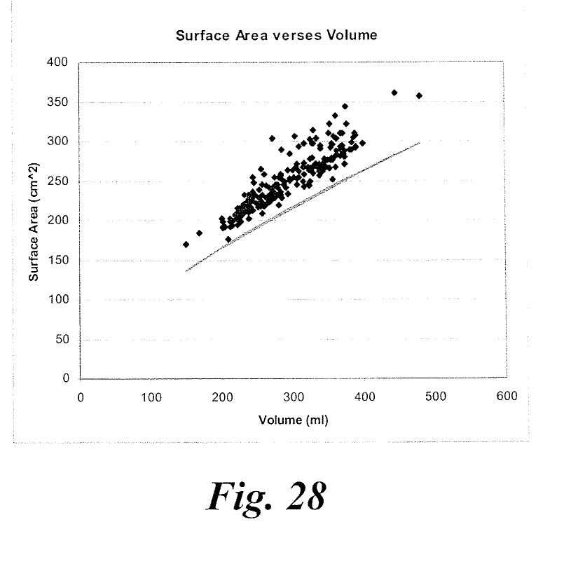

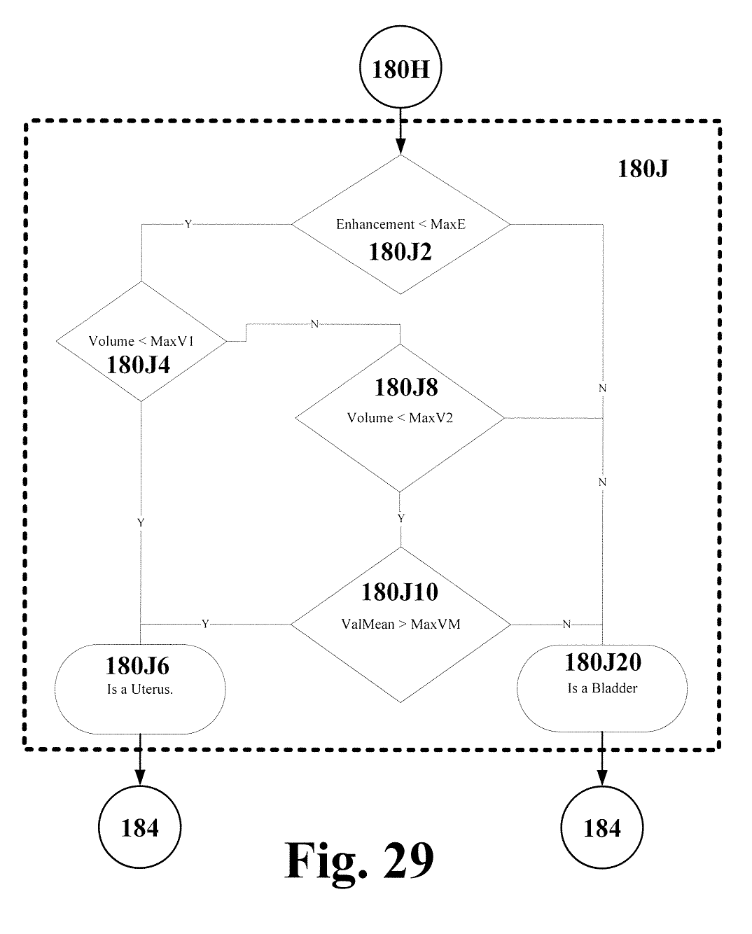

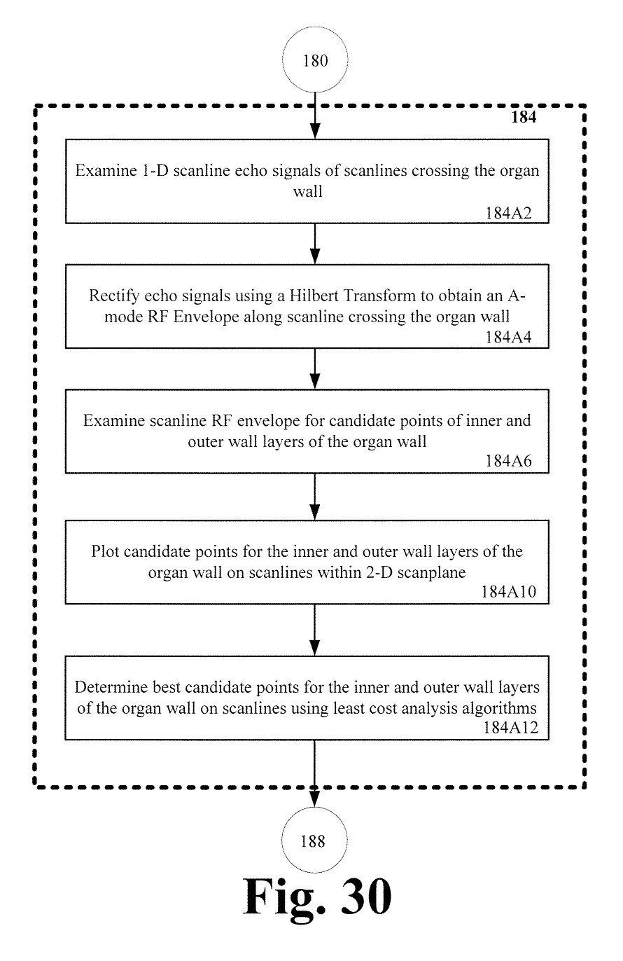

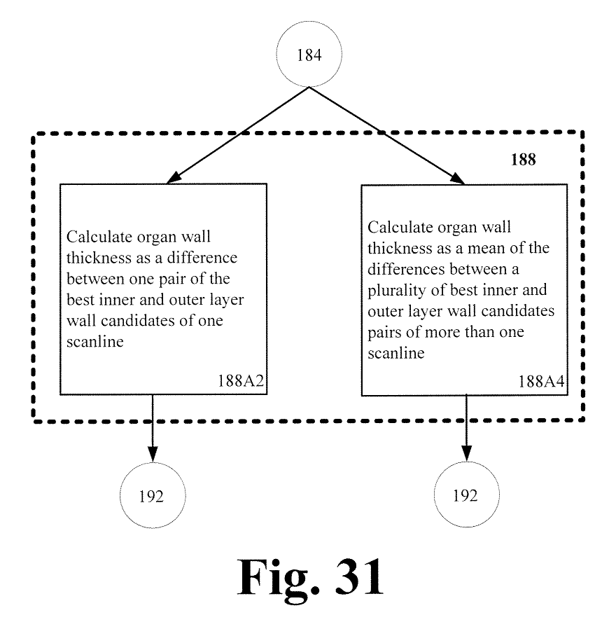

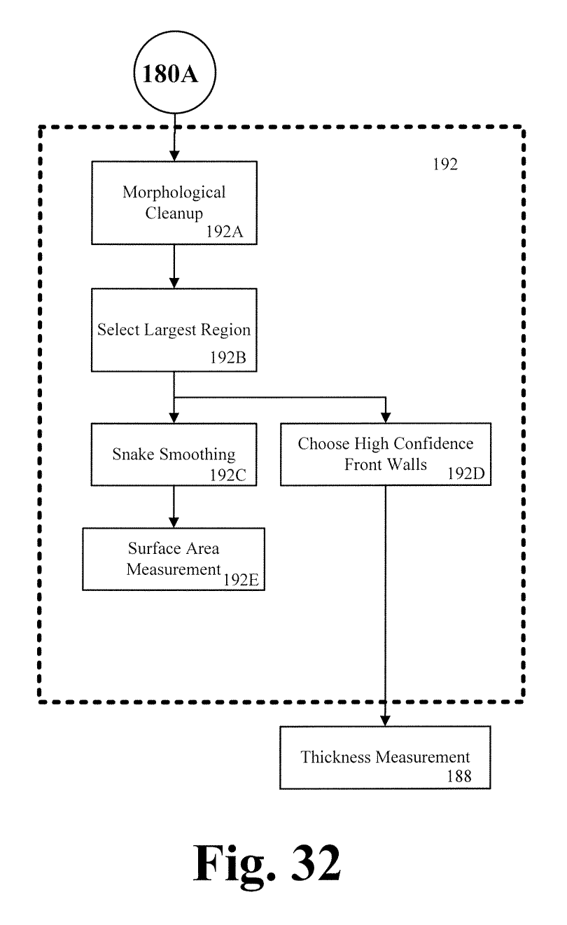

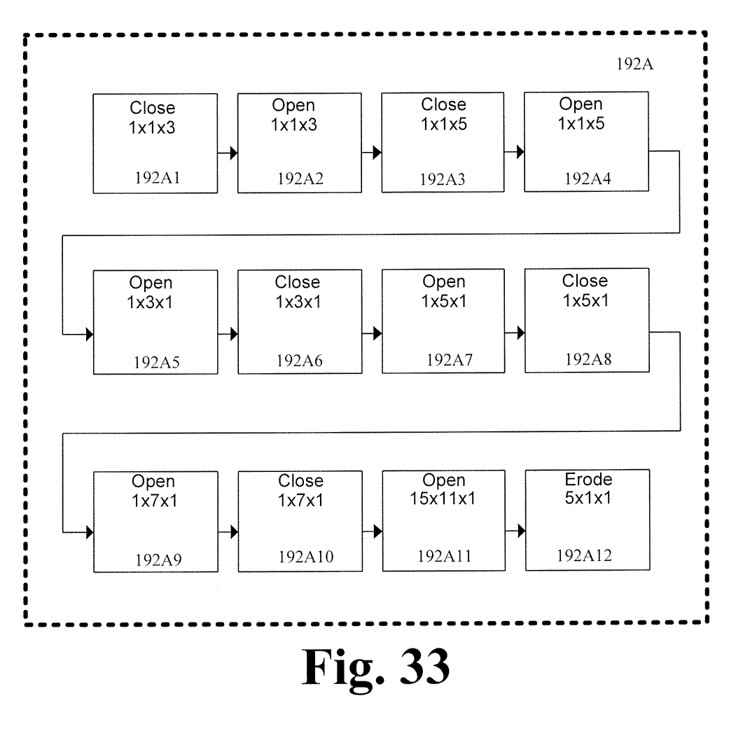

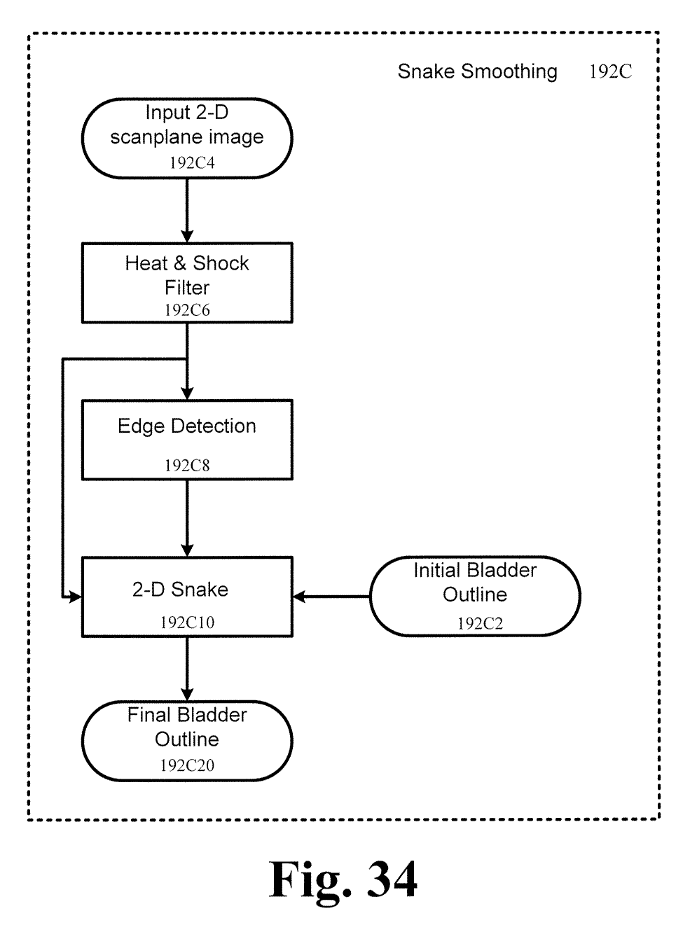

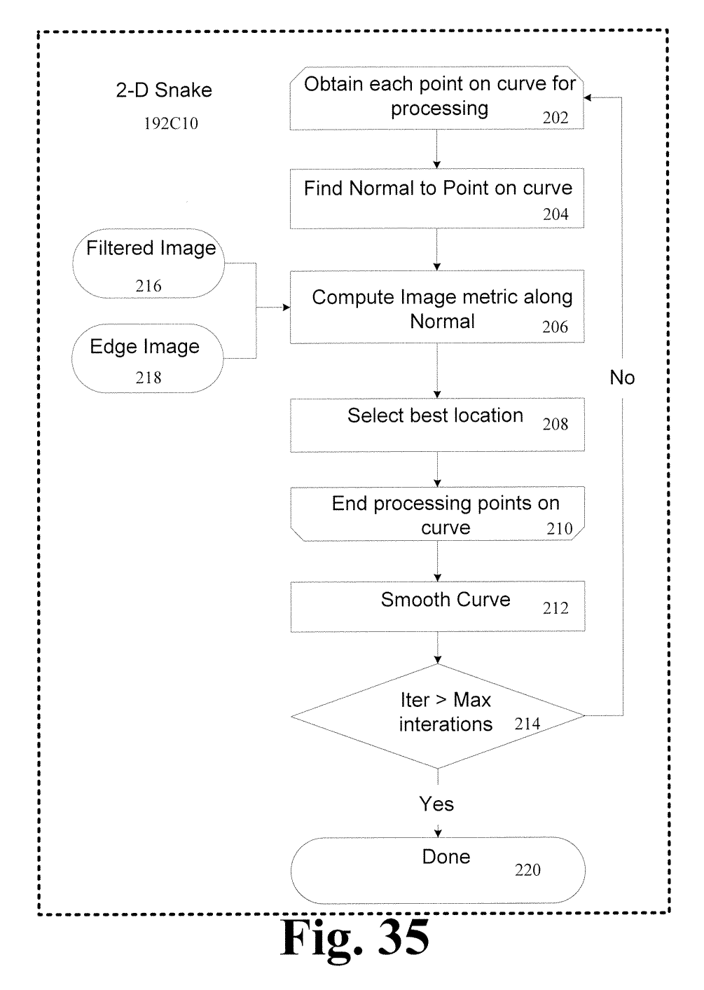

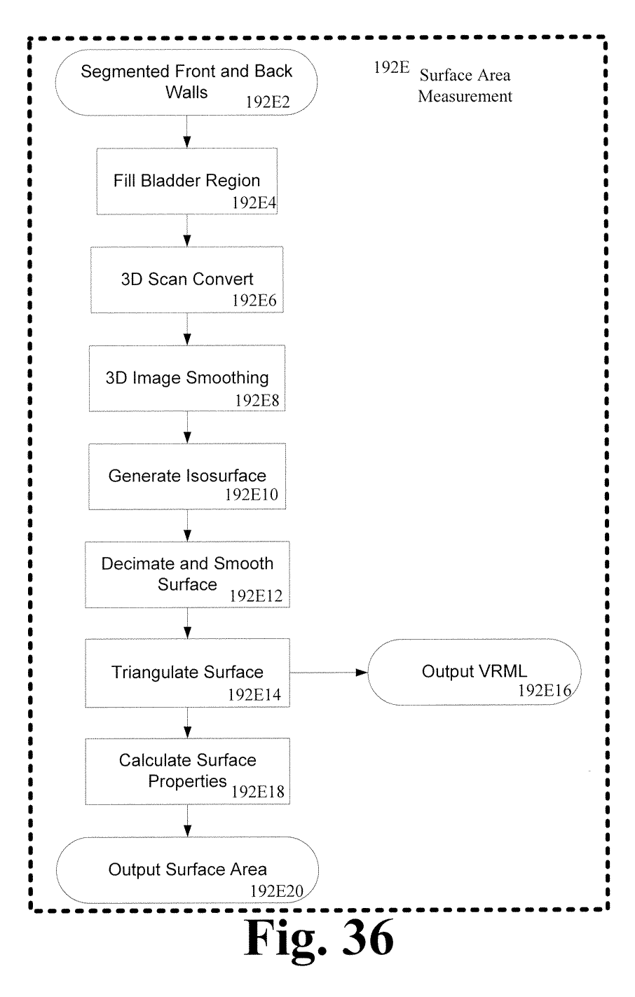

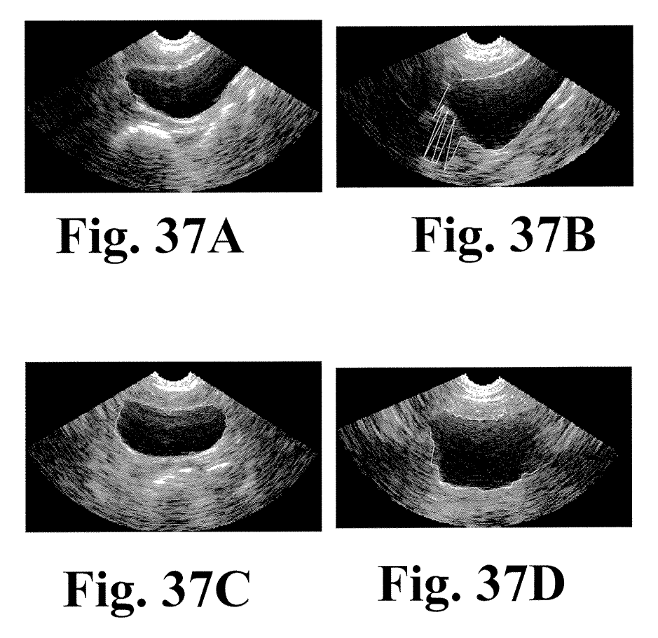

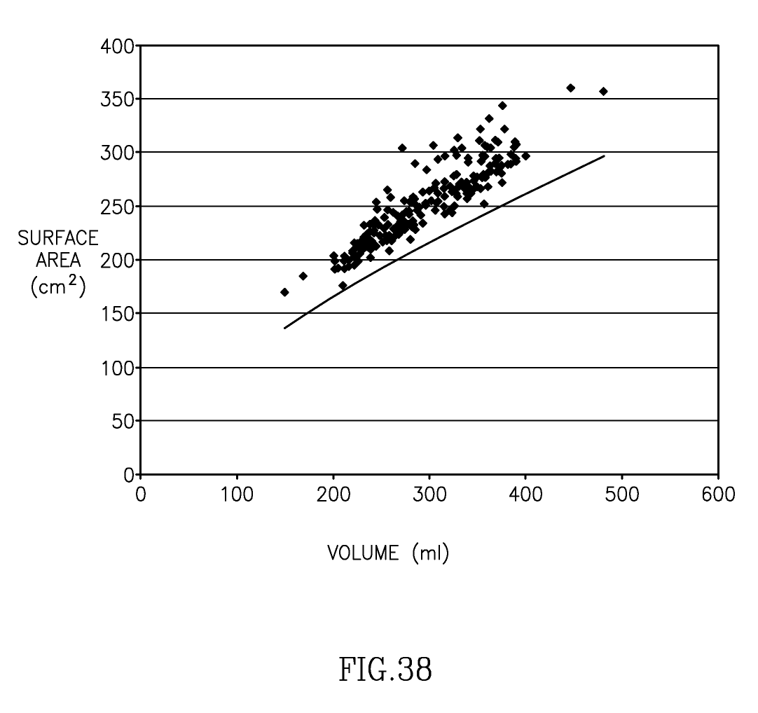

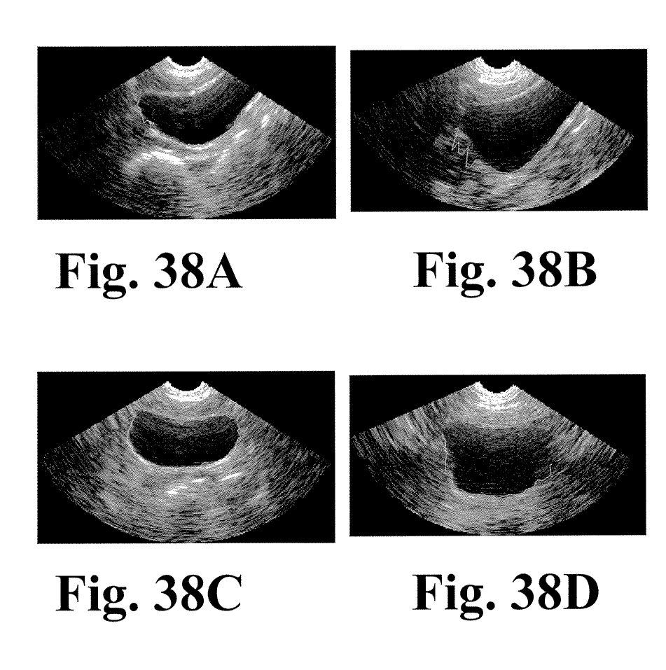

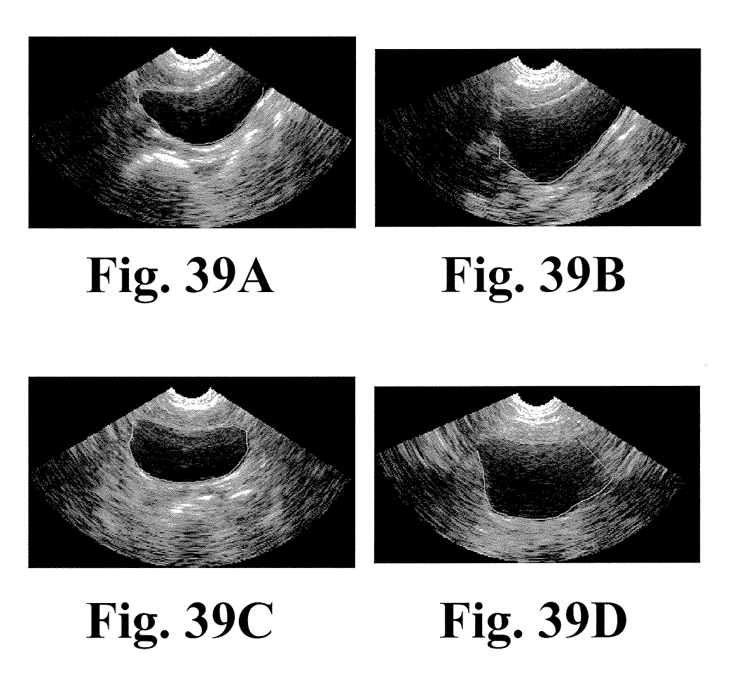

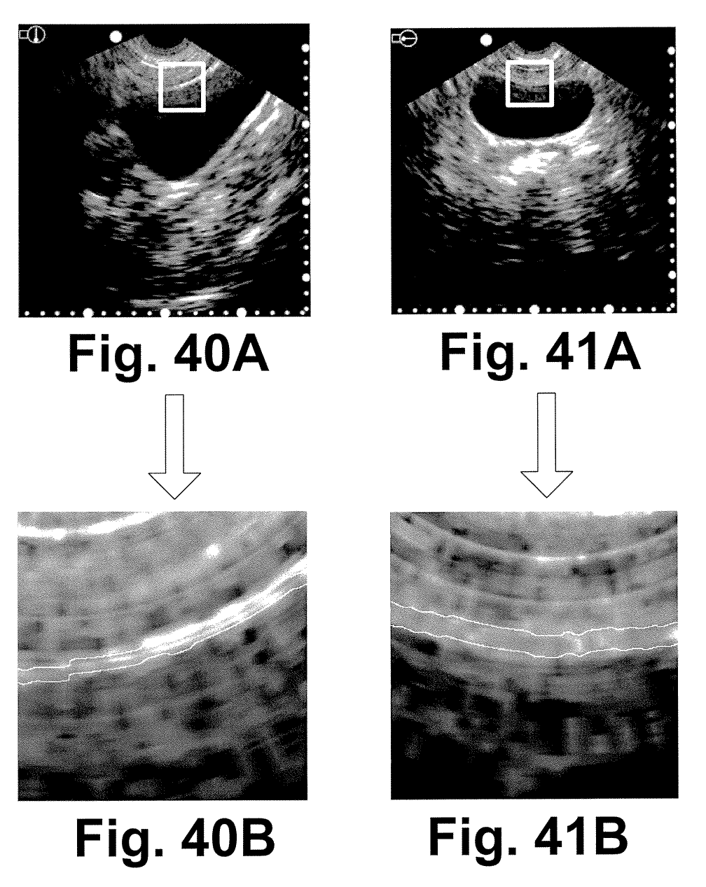

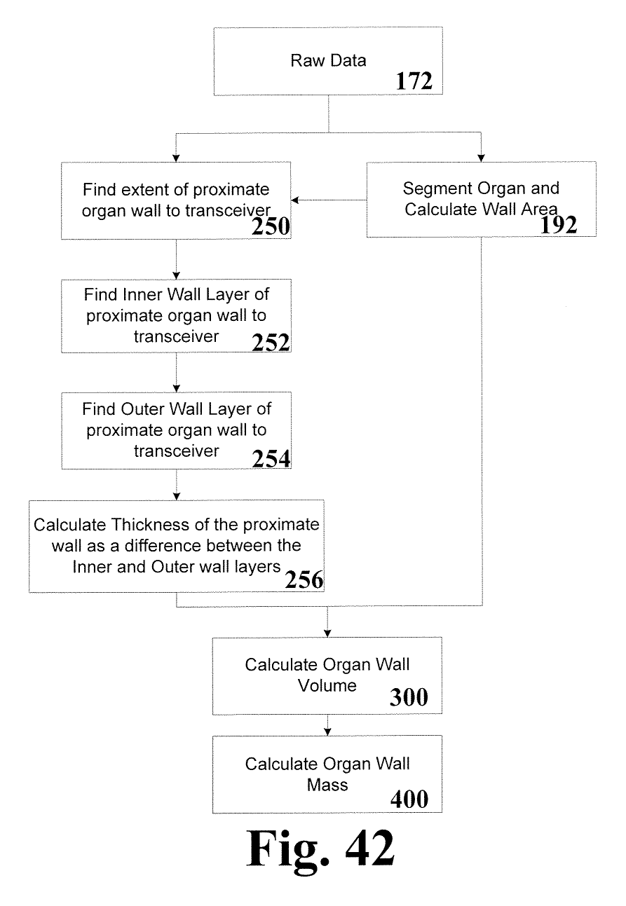

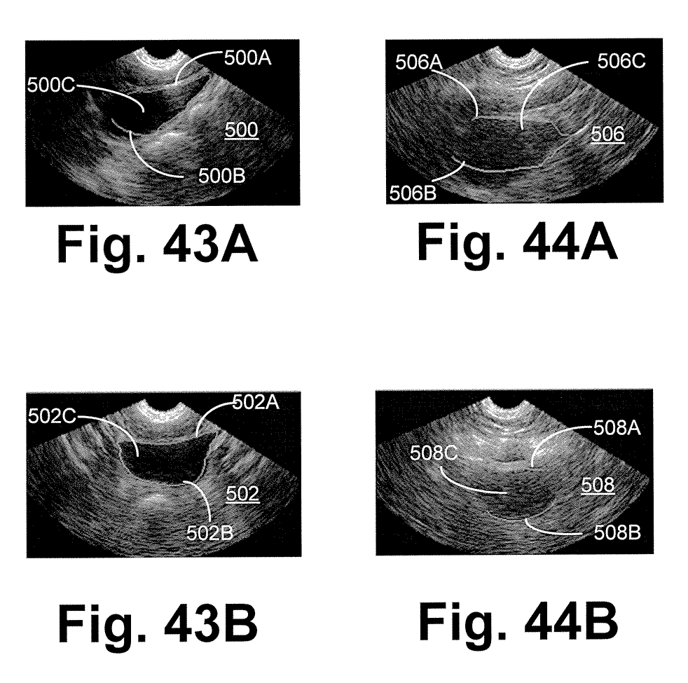

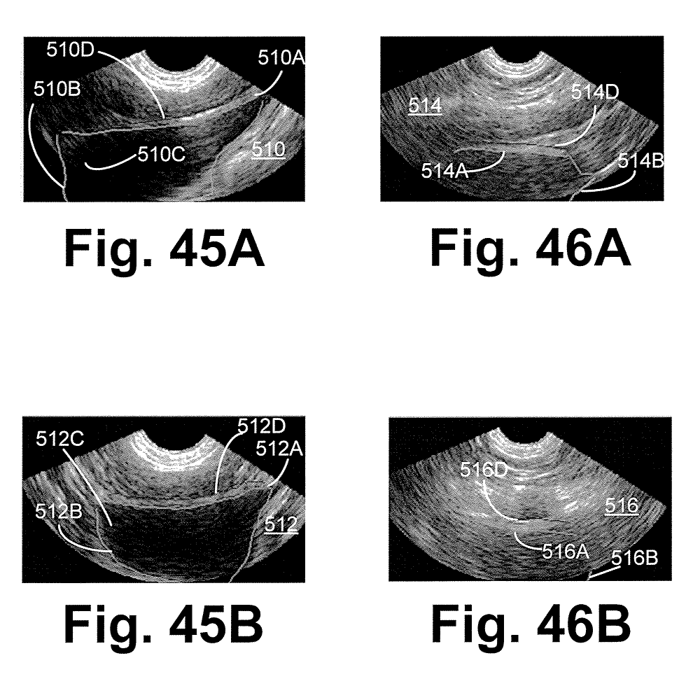

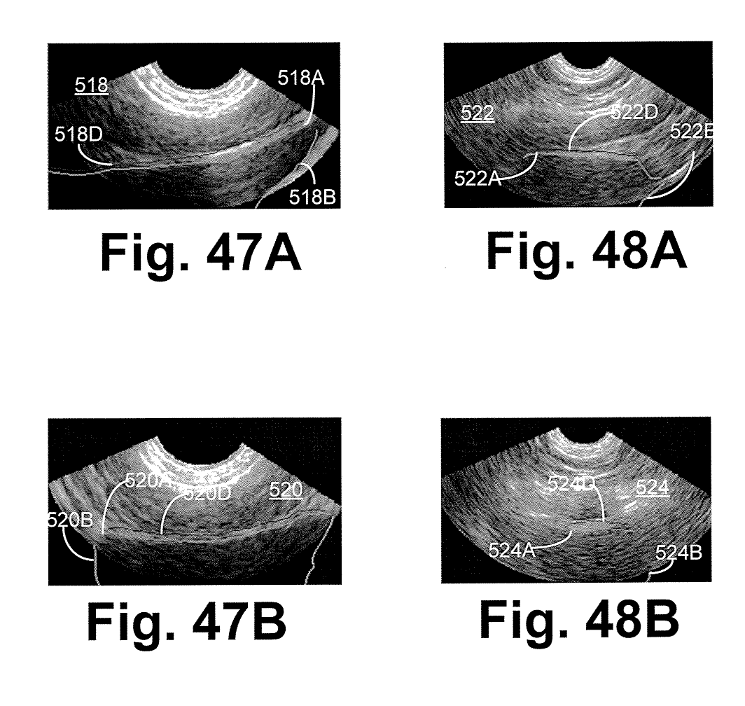

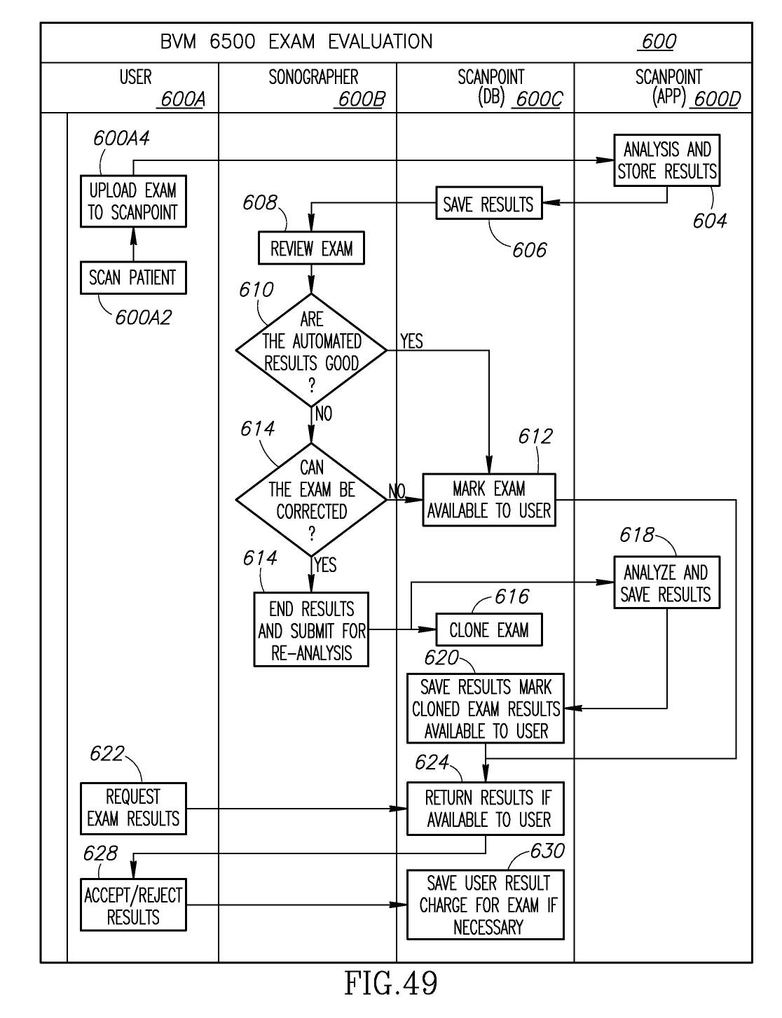

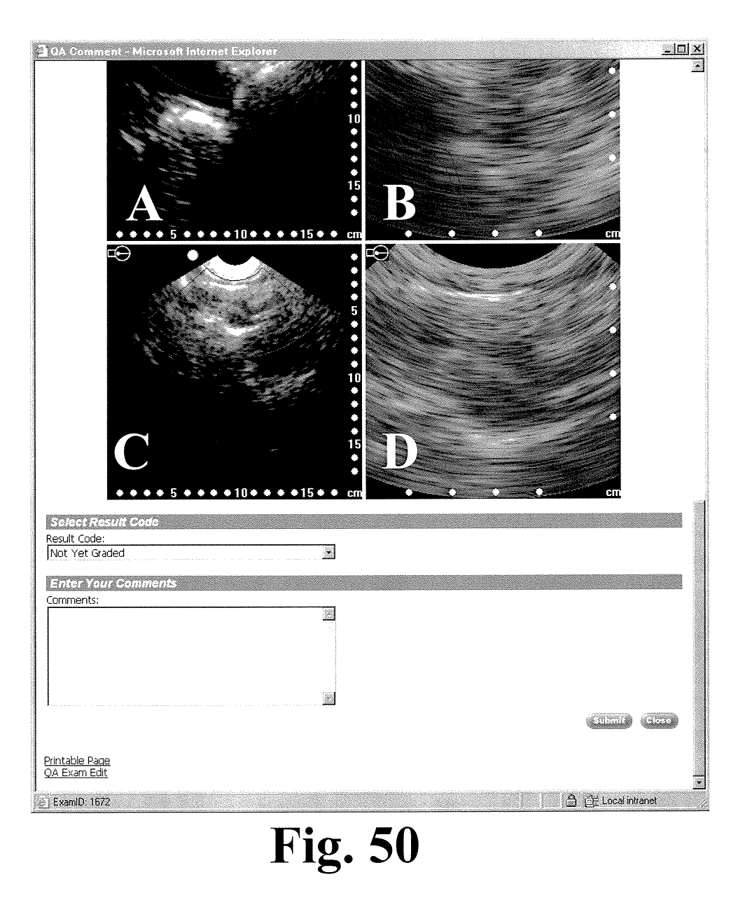

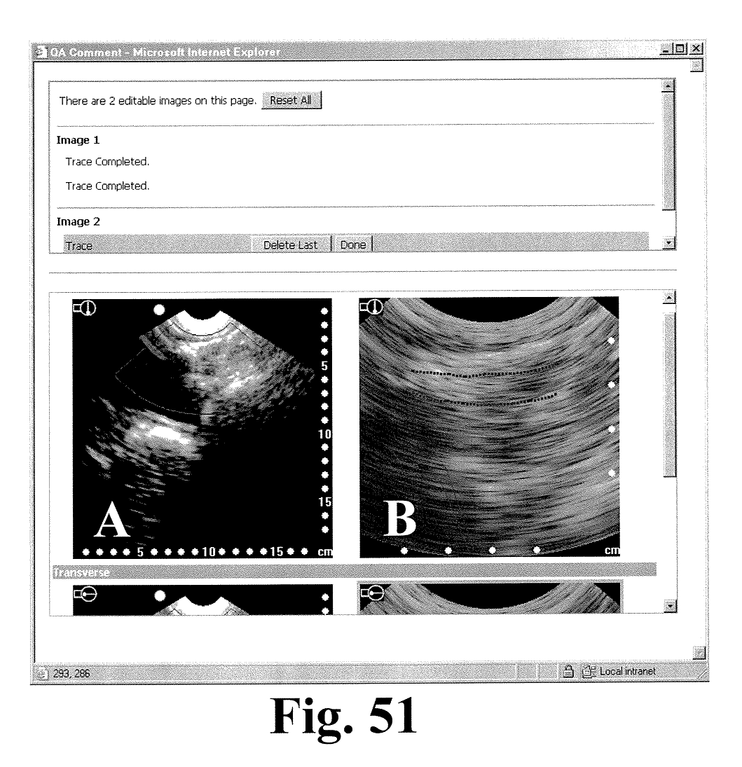

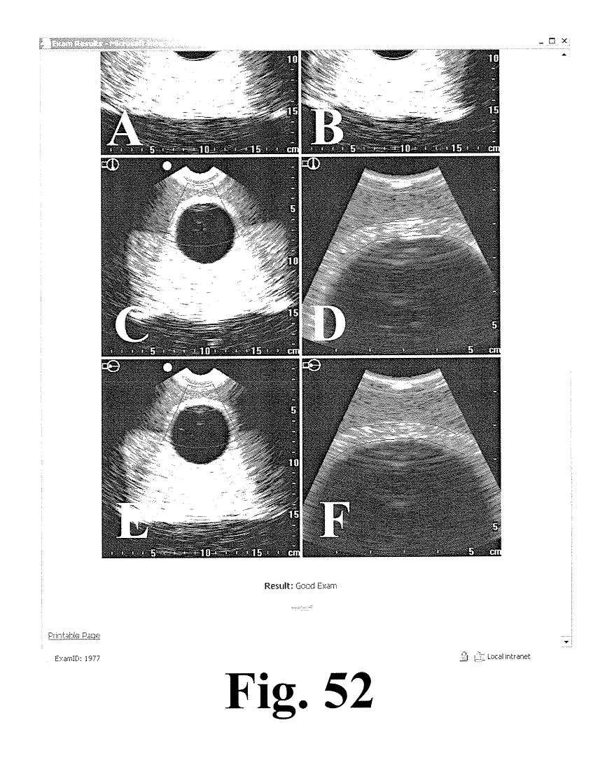

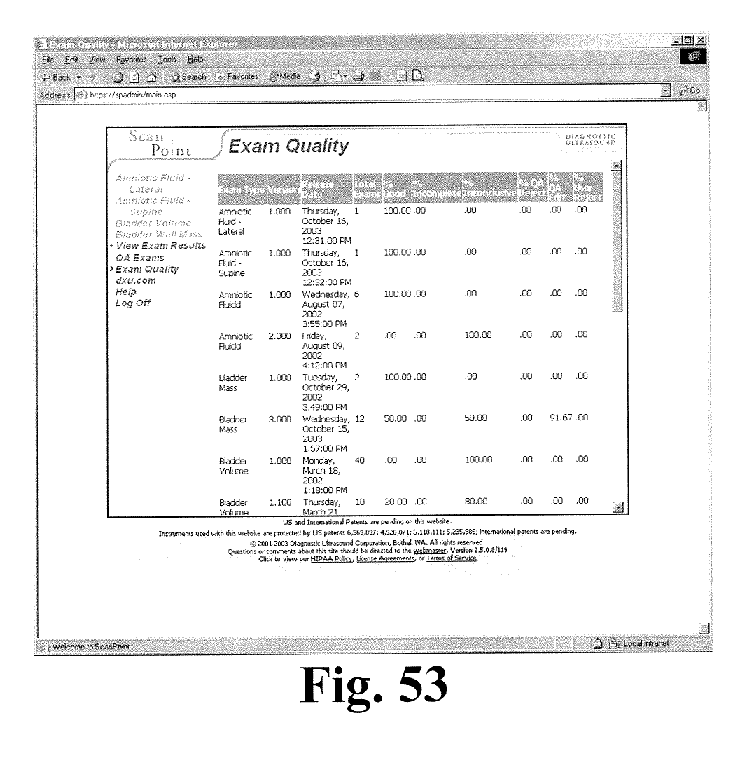

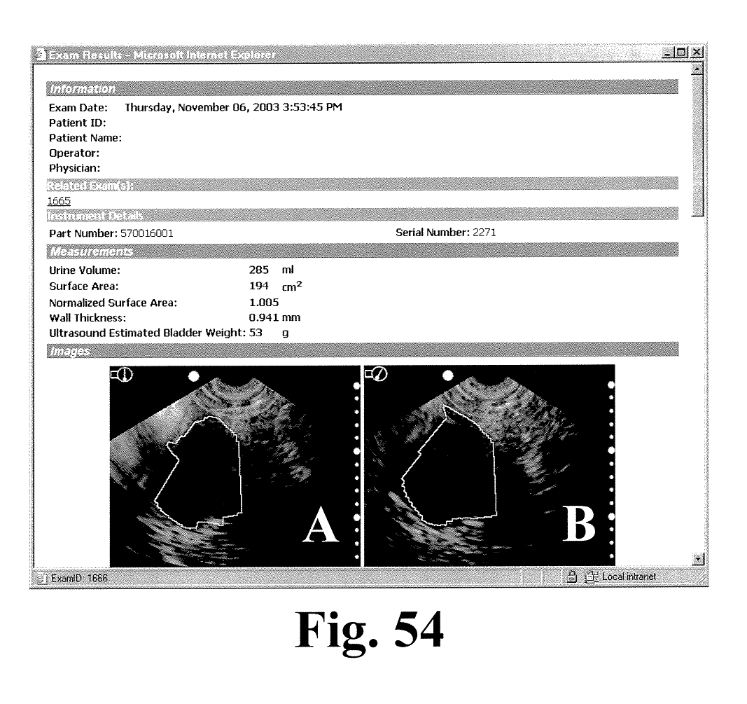

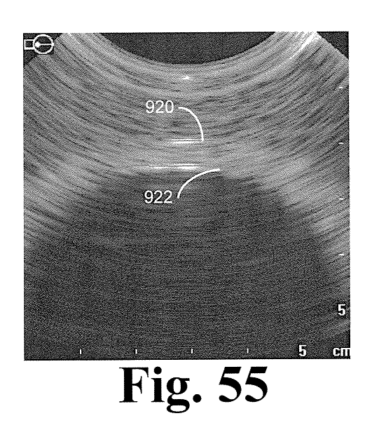

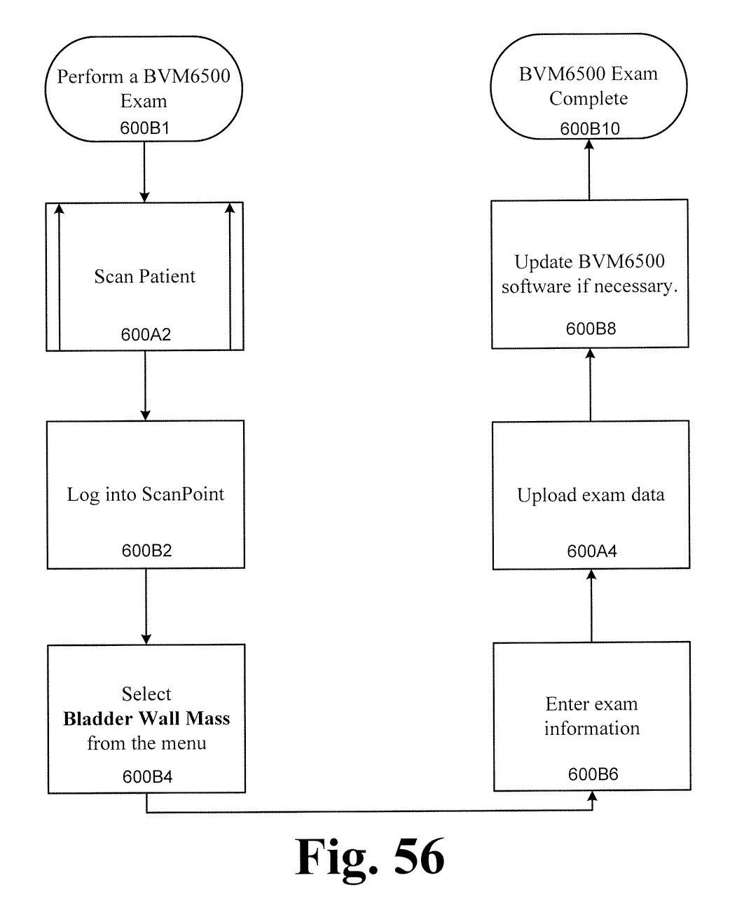

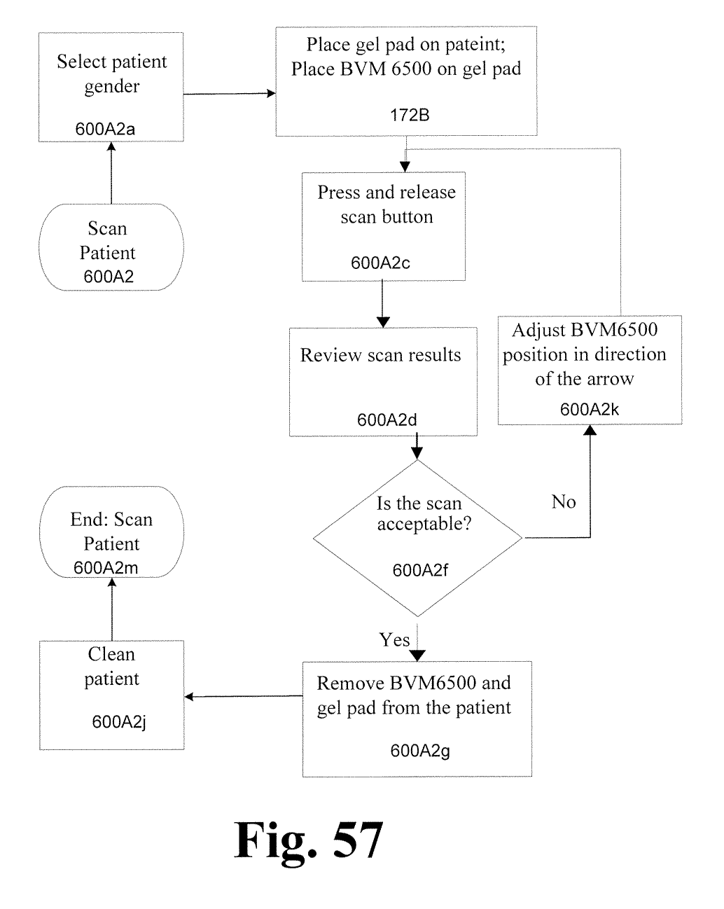

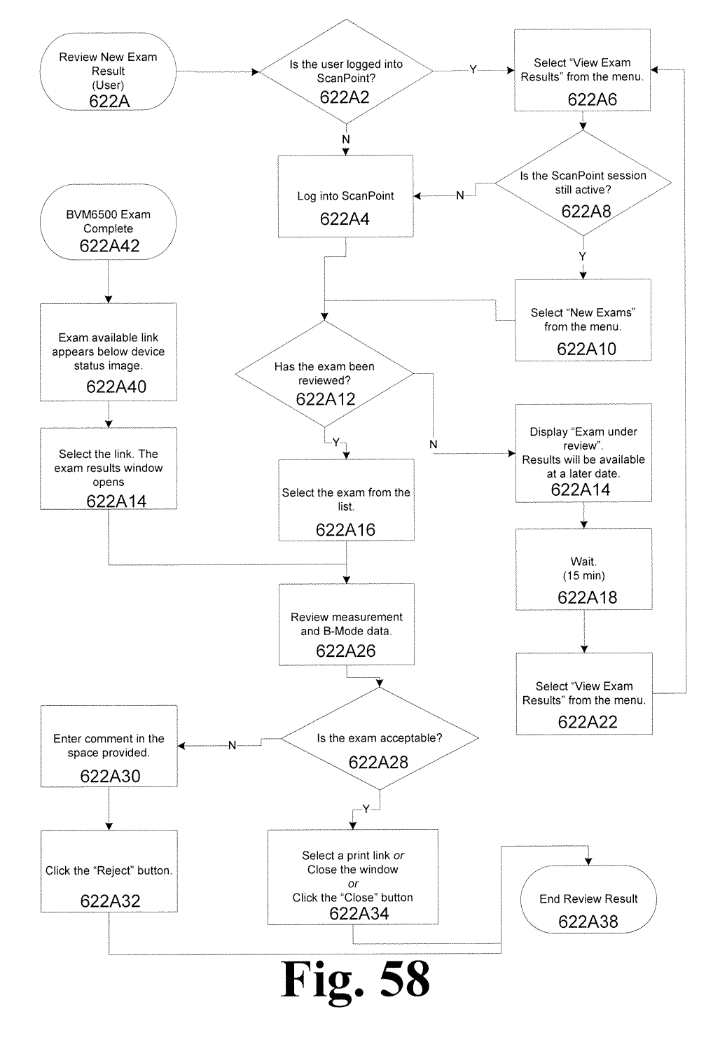

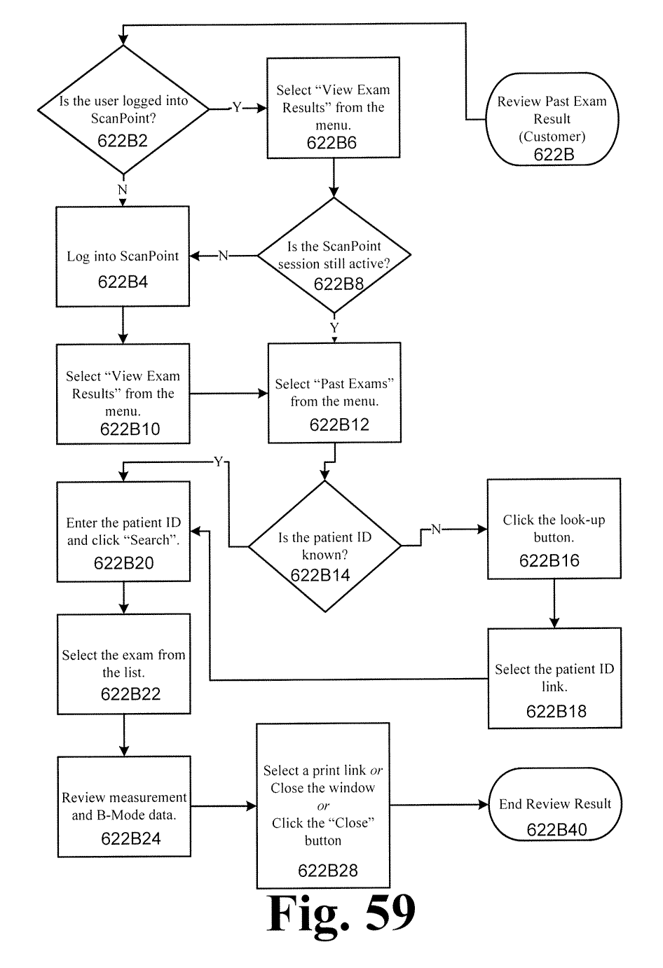

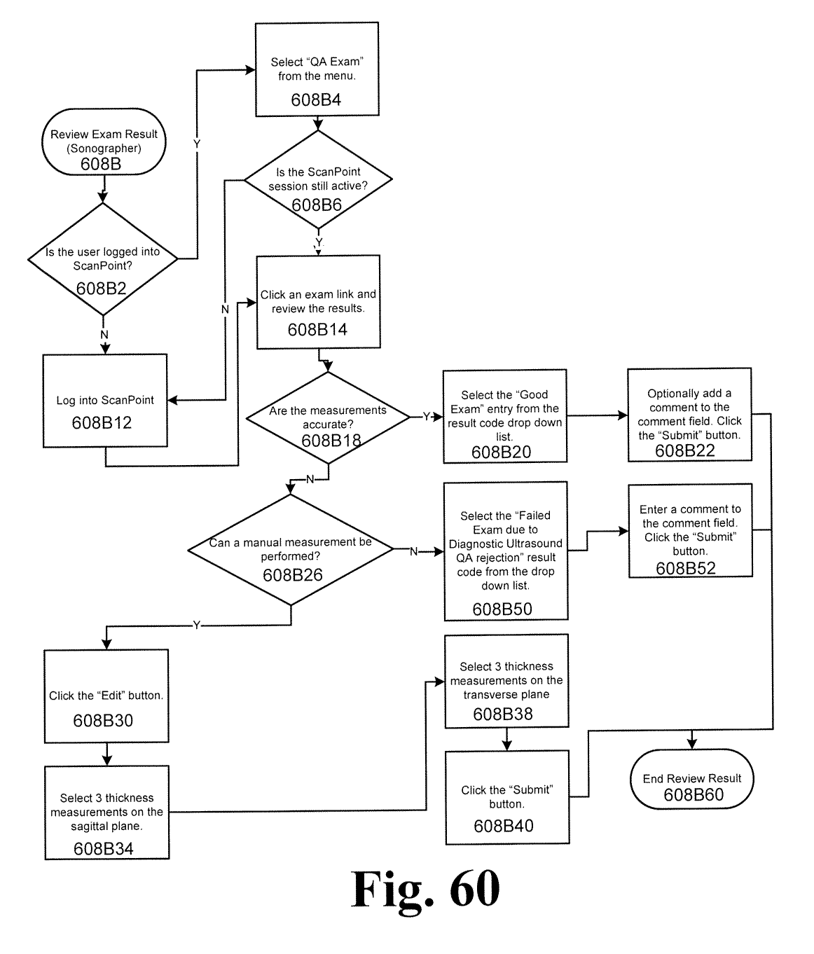

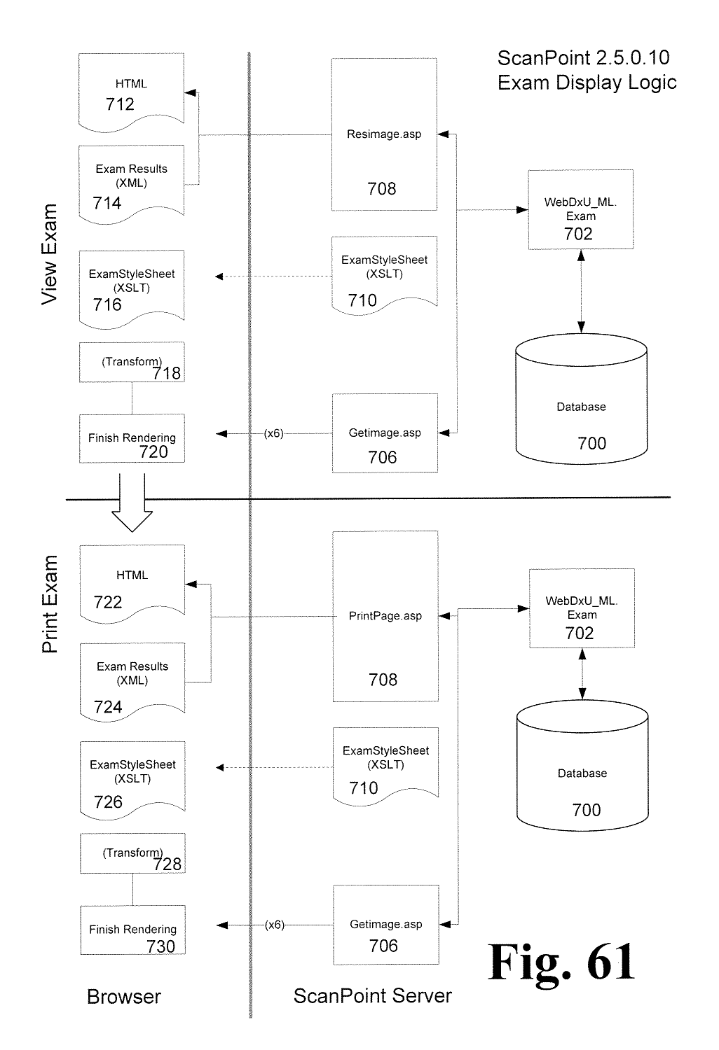

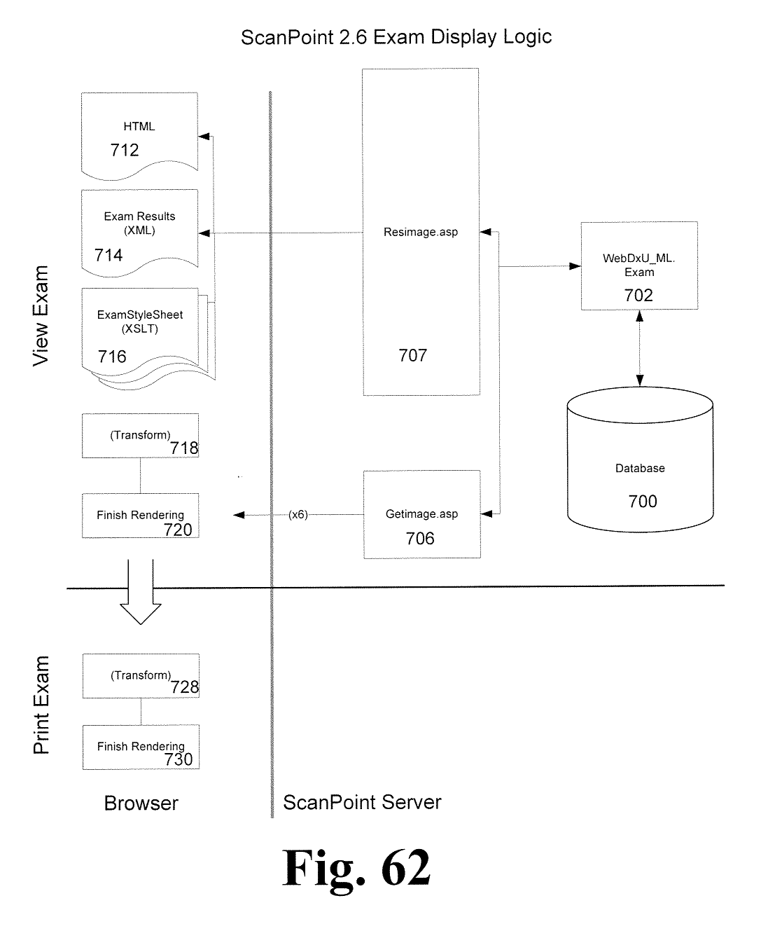

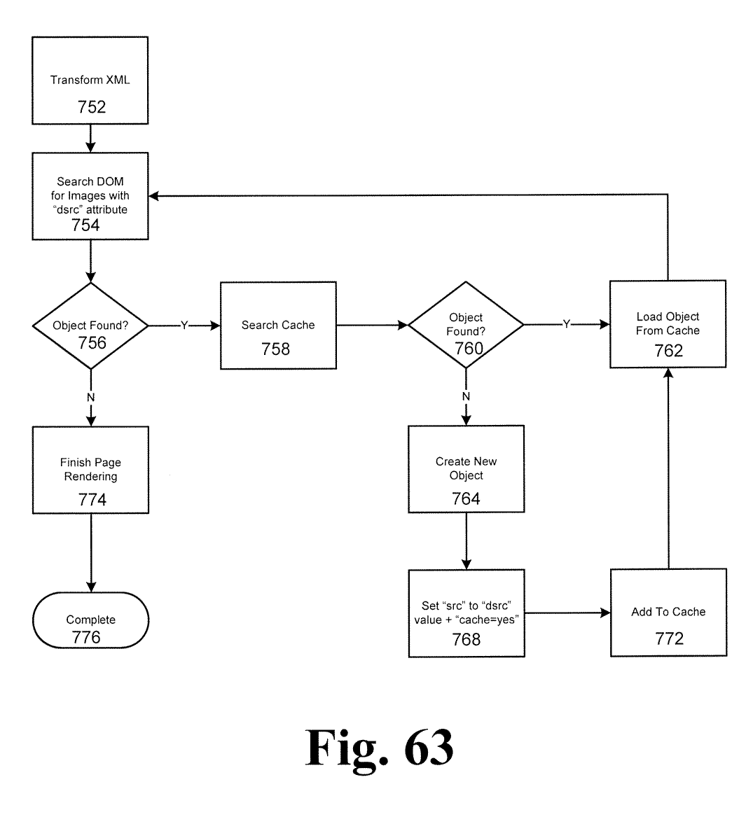

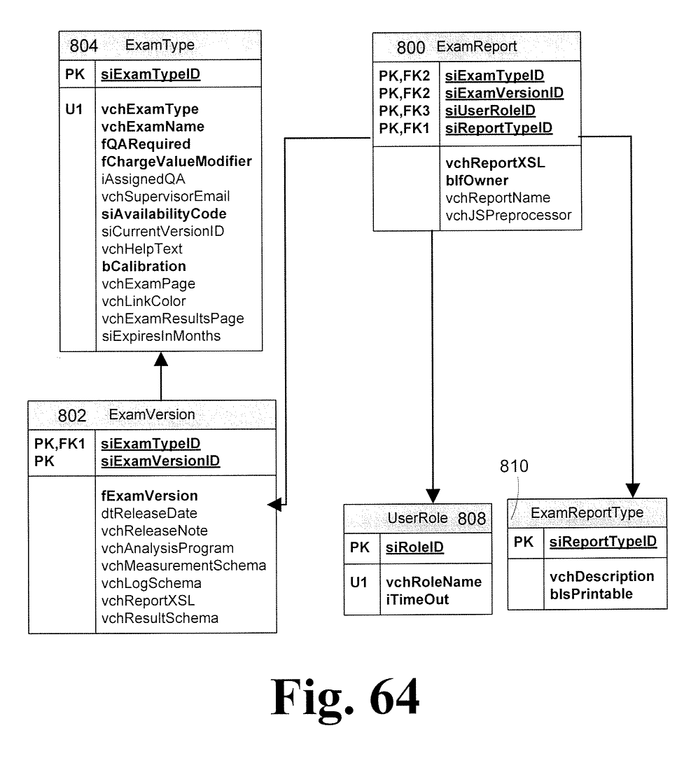

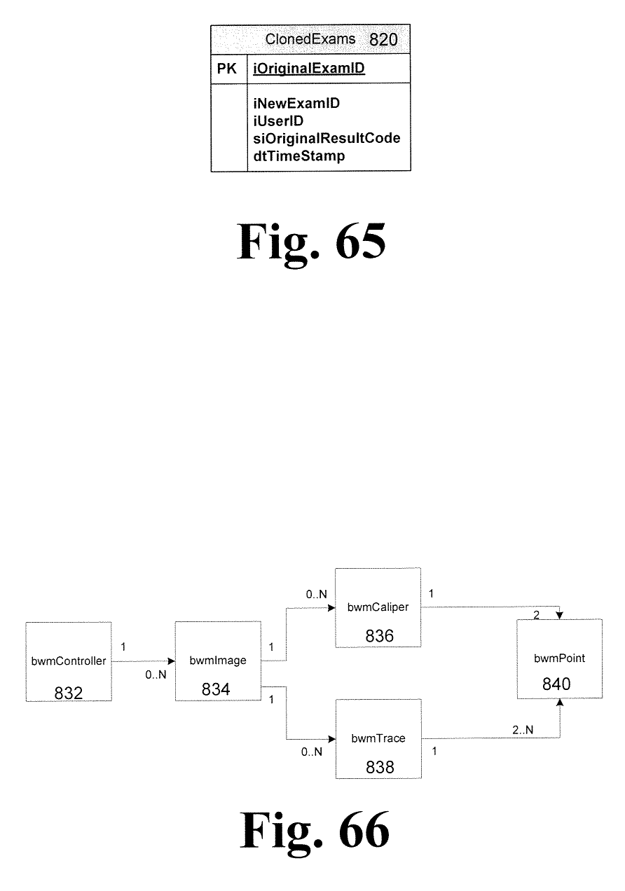

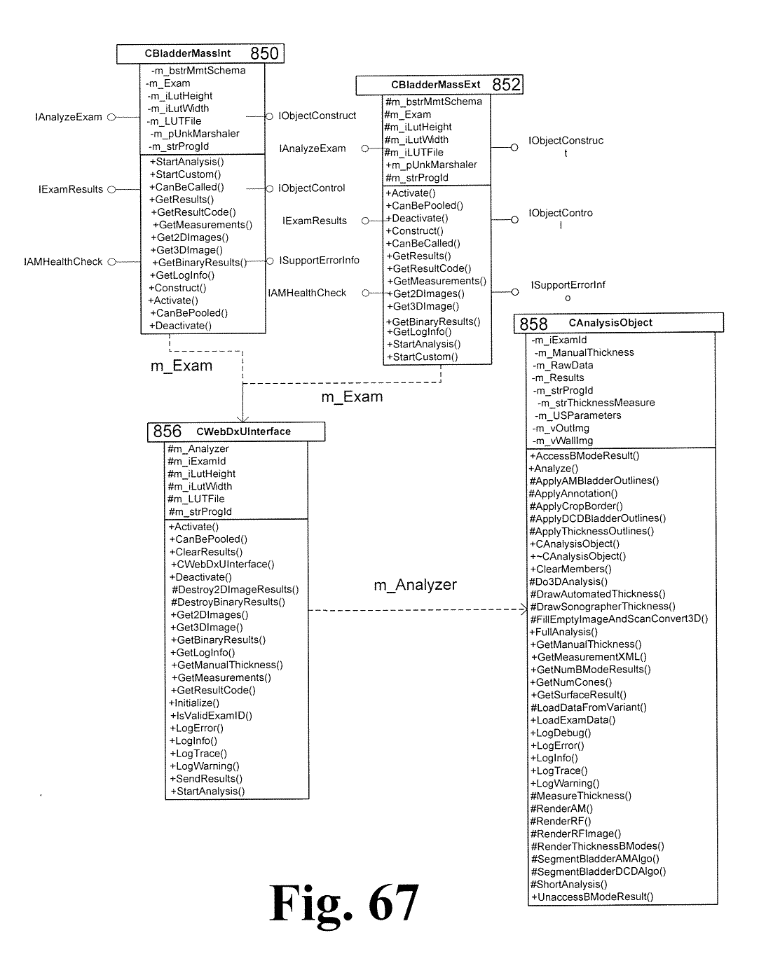

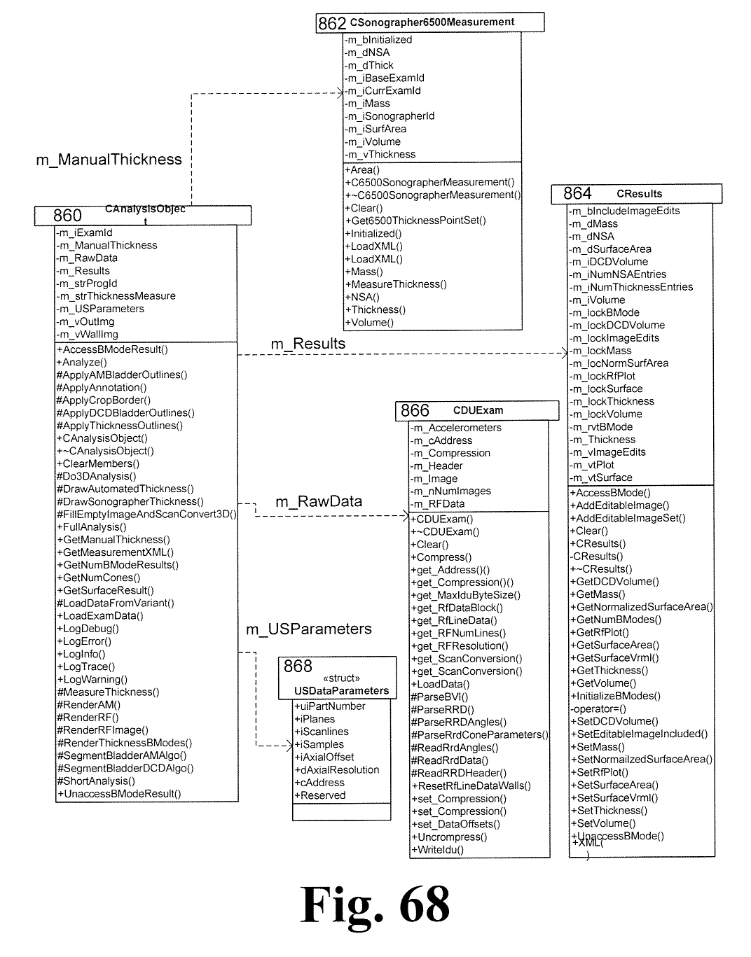

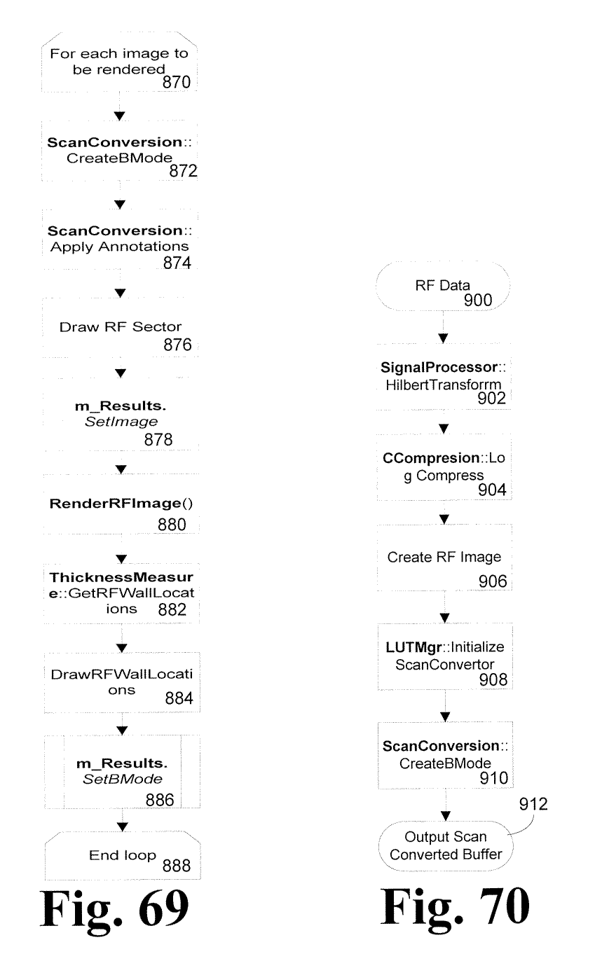

FIGS. 9A through 9D are four exemplary and sequential ultrasound images obtained from a male subject during an ultrasound examination;

FIGS. 10A through 10D are four exemplary and sequential ultrasound images obtained from a female subject during an ultrasound examination;

FIG. 11 is an exemplary, non-rectified echogenic signal received along a selected scanline during ultrasound imaging of a bladder;

FIG. 12 is an exemplary processed echogenic signal pattern from the selected scanline of the bladder imaging of FIG. 15;

FIG. 13 is the processed echogenic signal pattern of FIG. 12 that further shows a waveform that is generated by additional processing of the rectified waveform;

FIG. 14 is a method algorithm of the particular embodiments;

FIG. 15 is a flowchart that describes a method for scanning a bodily organ, according to an embodiment of the invention;

FIG. 16 is a diagram that describes a method for determining incident angles;

FIG. 17 is an idealized diagram of an echogenic envelope having an intensity pattern that crosses a front bladder organ wall;

FIG. 18 is an envelope of a scanline having an echogenic intensity distribution that crosses highly reflective adipose;

FIG. 19 is a B-mode ultrasound image that shows a family of wall layer locations corresponding to the candidate points of FIG. 18;

FIG. 20 is a diagrammatic view of a plurality of candidate wall points that result from an echogenic distribution;

FIG. 21A is a flowchart of a method for identifying an outer wall location according to an embodiment of the invention;

FIG. 21B is a flowchart of a method for identifying an inner wall location according to an embodiment of the invention;

FIG. 22 is an exemplary graph of a cost function generated along a selected scanline;

FIG. 23 is an exemplary scanplane of an internal anatomical region having a sector of scanlines superimposed on the scanplane;

FIG. 24 is an expanded portion of the scanplane 42 of FIG. 24 that shows the initial front wall location in greater detail;

FIG. 25 is an expansion of the sub-algorithm 172 of FIGS. 14 and 15;

FIG. 26 is an expansion of the sub-algorithm 180 of FIG. 14;

FIG. 27 is an expansion of the sub-algorithm 180A of FIG. 26;

FIG. 28 is an expansion of the sub-algorithm 180C of FIG. 26;

FIG. 29 is an expansion of the sub-algorithm 180J of FIG. 26;

FIG. 30 is an expansion of the sub-algorithm 184 of FIG. 14;

FIG. 31 is an expansion of the sub-algorithm 188 of FIG. 14;

FIG. 32 is an expansion of the sub-algorithm 192 of FIG. 31;

FIG. 33 is an expansion of the sub-algorithm 192A of FIG. 32;

FIG. 34 is an expansion of the sub-algorithm 192C of FIG. 32;

FIG. 35 is an expansion of the sub-algorithm 192C10 of FIG. 34;

FIG. 36 is an expansion of the sub-algorithm 192E of FIG. 32;

FIGS. 37A-D are B-mode scans overlaid with interface tracings;

FIGS. 38A-D are B-mode scans overlaid with interface tracings;

FIGS. 39A-D are B-mode scans overlaid with interface tracings;

FIGS. 40A-B are normal and magnified B-mode scans overlaid with interface tracings;

FIGS. 41A-B are normal and magnified B-mode scans overlaid with interface tracings;

FIG. 42 is an alternative-algorithm of FIG. 15;

FIGS. 43A-B are B-mode scans overlaid with interface tracings;

FIGS. 44A-B are B-mode scans overlaid with interface tracings;

FIGS. 45A-B are B-mode scans overlaid with interface tracings;

FIGS. 46A-B are B-mode scans overlaid with interface tracings;

FIGS. 47A-B are B-mode scans overlaid with interface tracings;

FIGS. 48A-B are B-mode scans overlaid with interface tracings;

FIG. 49 is a method algorithm for the Internet System;

FIG. 50 is a screen shot of four image panels;

FIG. 51 is a screen shot of two image panels;

FIG. 52 is a screen shot of six image panels;

FIG. 53 is a screen shot of Exam Quality Report;

FIG. 54 is a screen shot of two image panels; and

FIG. 55 is a scanplane image overlaid with inner and outer wall tracings using algorithms of the Internet System.

FIGS. 1A-D depicts a partial schematic and a partial isometric view of a transceiver, a scan cone comprising a rotational array of scan planes, and a scan plane of the array;

FIG. 2 depicts a partial schematic and partial isometric and side view of a transceiver, and a scan cone array comprised of 3D-distributed scan lines;

FIG. 3 depicts an ultrasound transceiver housed in a communications cradle and the data being wirelessly uploaded;

FIG. 4 depicts an ultrasound transceiver housed in a communications cradle where the data uploaded by electrical connection;

FIG. 5 depicts images showing the abdominal area of a patient being scanned by a transceiver and the data being wirelessly uploaded to a personal computer during initial targeting of a region of interest (ROI);

FIG. 6 depicts images showing the patient being scanned by the transceiver and the data being wirelessly uploaded to a personal computer of a properly targeted ROI in the abdominal area;

FIG. 7 is a schematic illustration and partial isometric view of a networked ultrasound system 100 in communication with ultrasound imaging systems 60A-D;

FIG. 8 is a schematic illustration and partial isometric view of an Internet connected ultrasound system 110 in communication with ultrasound imaging systems 60A-D;

FIG. 9A is a B-mode ultrasound image of a bladder in a transverse section using the either of the transceivers 10A-C with 3.7 MHz pulse frequency from imaging systems 60A-D;

FIG. 9B is a close-up of the image in FIG. 9A showing the anterior bladder wall;

FIG. 9C is a log-compressed A-mode line of one scan line similar to scan line 48 through the bladder and illustrates the relative echogenic as a function of scan line position or depth through the bladder;

FIG. 10 is an algorithm for the calculation of UEBW from V-mode® ultrasound data;

FIG. 11 is an expansion of sub-algorithm 172 of FIG. 10;

FIG. 12A is an expansion of sub-algorithm 178 of FIG. 10;

FIG. 12B is an expansion of an alternate embodiment of sub-algorithm 178 of FIG. 10;

FIG. 13 is an expansion of the process data to delineate bladder sub-algorithm 178-8 of FIGS. 12A and 12B;

FIG. 14 is an expansion of the Find Initial Walls sub-algorithm 178-8A of FIG. 13;

FIG. 15 is an expansion of the Fix Initial Walls sub-algorithm 178-8C of FIG. 13;

FIG. 16 is an expansion of surface area sub-algorithm 186 of FIG. 10 for sub-serosal layer 148;

FIG. 17 is an expansion of calculate surface area sub-algorithm 186 of FIG. 10 for sub-mucosal layer 146;

FIG. 18 is an expansion of the sub-algorithm 186-10 of FIG. 16 for sub-serosal layer 148;

FIG. 19 is an expansion of the sub-algorithm 186-20 of FIG. 17 for sub-serosal layer 146;

FIG. 20 is an expansion of calculate thickness sub-algorithm 192 of FIG. 10;

FIG. 21 is a set of three samples of bladder lumen delineations;

FIG. 22 are a first set of normal and magnified saggital images visualized by the ultrasound transceivers 10A-B;

FIG. 23 are a second set of normal and magnified saggital images visualized by the ultrasound transceivers 10A-B;

FIG. 24 is a schematic representation of four surface patch elements;

FIG. 25 is a schematic representation of three scan lines passing through the sub-serosal and sub-mucosal wall locations of an organ;

FIG. 26 depicts UEBW measurements for a subject group;

FIG. 27 depicts UEBW measurements for the subject group after excluding cases where the peritoneum merged with the subserosal layer of the bladder wall; and

FIG. 28 shows the bladder surface area calculated by particular method embodiments plotted against the bladder volume.

### DETAILED DESCRIPTION OF THE PREFERRED EMBODIMENT

The portable embodiment of the ultrasound transceiver of the present invention is shown in FIG. 1. The transceiver 10 includes a handle 12 having a trigger 14 and a gender changer 16, a transceiver housing 18 attached to the handle 12, a transceiver dome 20 and a display 24 for user interaction, attached to the transceiver housing 18 at an end opposite the transceiver dome 20. The transceiver 10 is held in position against the body of a patient by a user. In operation, the transceiver transmits a radio frequency ultrasound signal within the 2 to 10 MHz range to the body and then receives a returning echo signal. The returning echo signal provides an image signal for image processing. The gender changer 16 serves to adjust the delivery and reception of radio frequency ultrasound to the anatomy of a male patient and to the anatomy of a female patient. The transceiver is controlled by a microprocessor and software associated with the microprocessor and a digital signal processor of a computer system. As used in this invention, the term “computer system” broadly comprises any microprocessor-based or other computer system capable of executing operating instructions and manipulating data, and is not limited to a traditional desktop or notebook computer. The display 24 presents alphanumeric data indicating the proper or optimal positioning of the transceiver 10 for initiating a series of scans. In alternate embodiments, the two- or three-dimensional image of a scanplane may be presented in the display 24 of FIG. 1.

Although the preferred ultrasound transceiver is described above and depicted in FIG. 1, other transceivers may also be used. For example, the transceiver need not be battery-operated or otherwise portable, need not have a top-mounted display 24, and may include many other features or differences. The transceiver 10 need only be able to non-invasively probe within the body to gather data that can be used to analyze internal objects such as the bladder. The display 24 may be a liquid crystal display (LCD), a light emitting diode (LED), a cathode ray tube (CRT), or any suitable display capable of presenting alphanumeric data or graphic images.

Once optimally positioned over the abdomen for scanning, the transceiver 10 transmits an ultrasound signal (approximately 3.7 MHz in the preferred embodiment, and commonly in a 2-10 MHZ range) into the bladder region. The ultrasound signal is in the form of generally linear signal bursts known as scanlines, as illustrated in FIG. 2. The scanlines, each approximately 20 cm long, originate from the transceiver dome 20, producing a dome cutout 30 in a cluster of scanlines forming a scanplane 32. Within the scanplane 32 are a plurality of scanlines that share a common rotational angle (θ), but have a unique tilt angle (φ). In the preferred embodiment, each plane contains 77 scan lines, although the number of lines can vary within the scope of this invention, and the angular separation between the lines can vary within the scope of this invention.

The angular separation or spacing between lines may be uniform (substantially equal angular spacings, say 1.5° between each scanline) or non-uniform (substantially unequal angular spacings). An example of non-uniform angular spacing would be “1.5-6.8-15.5-7.2-so on” sequence where 1.5° is between a first line and a second line, 6.8° is between the second line and a third line, 15.5° is between the third line and a fourth line, 7.2° is between the fourth line and a fifth line, and so on. The angular separation may also be a combination of uniform and non-uniform angular spacings, for example a sequence of “1.5-1.5-1.5-7.2-14.3-20.2-8.0-8.0-8.0-4.3-7.8-so on” angular spacings.

After a plane of scanlines is transmitted, the transceiver rotational angle θ is incremented slightly and another plane of pulse-echo signals are transmitted and received to form a new scanplane. This process is repeated as desired, producing a series of scanplanes in which each plane will be slightly rotated from the prior plane by a selected rotational angle θ interval. The rotational angle θ interval or spacing between scanplanes can be uniform or nonuniform. Uniform intervals between scanplanes have approximately the same degrees separating each scanplane from its nearest neighbors. For example, as shown in FIG. 3, in the preferred embodiment each scanplane 32 is transmitted, received, and displayed into a twenty-four plane array, with approximately 7.5° rotational angle θ interval separating each scanplane from its nearest neighbors in the array. In contrast, an example of non-uniform intervals between scanplanes in an array having a sequence “3.0-18.5-10.2-so on” would be a rotational angle θ interval of 3.0° between a first and a second scanplane, a θ interval of 18.5° between the second scanplane and a third scanplane, then a θ interval of 10.2° between the third scanplane and a fourth scanplane, and so on. The scanplane interval may also be a combination of uniform and non-uniform rotational angle θ intervals, for example a sequence of “3.0-3.0-3.0-18.5-10.2-20.6-7.5-7.5-7.5-16.0-5.8-so on” θ intervals. Also illustrated in FIG. 3 is the tilt angle φ that sweeps through angles between −60° and 60° for a total of 120°.

FIG. 4 presents a top view of a twenty-four plane array, the twenty-four array having a uniform rotational angle θ between each scanplane. The number of scanplanes in the array is at least two, but can be varied above two. The rotational angle θ intervals between scanplanes in an array can be varied, and be uniform and non-uniform.

For wedge and translational arrays, the scanplanes may similarly be uniformly spaced, non-uniformly spaced, or a combination of uniformly spaced and non-uniformly spaced scanplanes.

As the scanlines are transmitted and received, the returning echoes are changed into analog electrical signals by a transducer, converted to digital signals by an analog-to-digital converter, and conveyed to the digital signal processor of the computer system for analysis to determine the locations of the bladder walls. The computer system itself is not depicted, but in a preferred embodiment includes a microprocessor and a RAM, hard-drive, optical drive, or other memory for storing processing instructions and data generated by the transceiver 10.

FIG. 5 is a graphical representation of a plurality of 3D-distributed scanlines emanating from the transceiver 10 forming a scan cone 35. The scan cone 35 is formed by a plurality of 3D distributed scanlines that comprises a plurality of internal and peripheral scanlines. The scanlines are one-dimensional ultrasound A-lines that emanate from the transceiver 10 at different coordinate directions, that taken as an aggregate, from a conic shape. The 3D-distributed A-lines (scanlines) are not necessarily confined within a scanplane, but instead are directed to sweep throughout the internal and along the periphery of the scan cone 35. The 3D-distributed scanlines not only would occupy a given scanplane in a 3D array of 2D scanplanes, but also the inter-scanplane spaces, from the conic axis to and including the conic periphery. The transceiver 10 shows the same illustrated features from FIG. 1, but is configured to distribute the ultrasound A-lines throughout 3D space in different coordinate directions to form the scan cone 35.

The internal scanlines are represented by scanlines 37A-C. The number and location of the internal scanlines emanating from the transceiver 10 is the number of internal scanlines needed to be distributed within the scan cone 35, at different positional coordinates, to sufficiently visualize structures or images within the scan cone 35. The internal scanlines are not peripheral scanlines. The peripheral scanlines are represented by scanlines 39A-F and occupy the conic periphery, thus representing the peripheral limits of the scan cone 35.

Once the wall locations are identified, the wall locations, demodulated magnitude data, and a subset of quadrature amplitude demodulated signal in the region of the anterior bladder wall are directed to the microprocessor for further analysis according to the algorithm illustrated in FIG. 6 for the preferred embodiment of the invention. First, ultrasound data is acquired relative to the bladder, as shown in the first block 50. In general, bladder-specific data can be acquired by a user who manipulates the transceiver 10 while viewing the received data on a display screen and then positioning the transceiver 10 as necessary so that the bladder is sufficiently within the field of view of the cone as depicted in FIG. 3, or within the field of view of the scan cone 35 depicted in FIG. 5.

After obtaining ultrasound bladder data, the ultrasound data is processed to determine if the bladder contains approximately 200 to approximately 400 ml, as shown in the second block 51. If “No”, then the bladder is allowed to accumulate approximately 200 to approximately 400 ml, as shown in the third block 52, or, if “Yes, meaning the bladder already contains the preferred approximate 200-400 ml volume, then the locations of the bladder walls, as shown in the fourth block 53, may be undertaken. The determination of organ wall locations and other such exterior boundaries within an ultrasound scan is within the capability of ultrasound devices presently on the market. In general, however, the process determines the length of a scanline from the transceiver dome to the bladder wall. The data, including wall locations, is stored in the computer memory.

Once the full cone of ultrasound magnitude data has been scanned and wall locations have been determined by the digital signal processor, the microprocessor further analyzes the data to correct any misdetection in wall location and to determine bladder volume. Two specific techniques for doing so are disclosed in detail in U.S. Pat. No. 4,926,871 to Ganguly et al and U.S. Pat. No. 5,235,985 to McMorrow et al, which are incorporated by reference. These patents provide detailed explanations for non-invasively transmitting, receiving and processing ultrasound signals relative to the bladder, and then for calculating bladder volume.

Using the methods provided by the '871 and '985 patents, the resultant data is used to determine whether or not the bladder volume is with a range of approximately 200 to approximately 400 ml. If the bladder volume is within that range, the ultrasound data is used to determine the actual surface area from the wall locations, as indicated in the fifth block 54. The surface area calculation is explained in greater detail below. While calculating the surface area in the fifth block 54, reflected RF ultrasound waves are received from the anterior bladder wall, as indicated in the sixth block 56. Although these tasks are preferably conducted in parallel, they may alternatively be processed in series. Thereafter, as shown in the seventh block 58, the bladder wall thickness is determined from the coherent signals that overlap at the wall locations. The determination of bladder wall thickness is explained in greater detail below. Finally, as shown in the seventh block 58, the bladder mass is computed as a product of thickness, area, and bladder density.

The volume restriction described in the previous paragraph defines the range of bladder volumes that enable an optimal measurement of the bladder mass. The mass calculation may be performed at a volume not in this range, but this will generally result in a less accurate measurement. For example, bladder volumes less than 200 ml and greater than 400 ml can be measured, but with less accuracy. For volumes substantially greater than 400 ml, for example bladder volumes of 1000 ml to multi-liters, the preferred embodiment will utilize scanlines greater than 20 cm to accommodate the larger bladder sizes. The preferred embodiment may be applied to measure the thicknesses and masses of internal organs of human and animals. The lengths of the scanlines are adjusted to match the dimensions of the internal organ to be scanned.

Surface area determination. The surface area measurement of fifth block 54 is performed by integrating the area of interpolating surface patch functions defined by the wall locations. The mathematical calculations are provided below in greater detail.

The surface of the bladder is defined to be S. This surface corresponds to the actual surface of the bladder determined by analysis of the wall locations of the bladder. Since this shape is not known in advance, modeling the bladder as a sphere or an ellipsoid provides only a crude approximation of the surface. Instead, the surface S is defined as a construction of a series of individual surface patches s_{i,j}, where i and j count through the latitude and longitude components of the surface, similar to the division of the Earth's surface into lines of latitude and longitude. The area of the bladder surface, S, is defined as the sum of all the individual surface patches, S=Σs_{i,j}.

As depicted in three dimensions in FIG. 7, by way of example, five scanplanes 32-48 are seen transmitted substantially longitudinally across a subserosal wall location 72 referenced to a tri-axis plotting grid 69. The five scanplanes include the first scanplane 32, a second scanplane 36, a third scanplane 40, a fourth scanplane 44, and a fifth scanplane 48. The scanplanes are represented in the preceding formulas as subscripted variable j. Substantially normal to the five longitudinal scanplanes are five latitudinal integration lines 60-68 that include a first integration line 60, a second integration line 62, a third integration line 64, a fourth integration line 66, and a fifth integration line 68. The integration lines are represented in the preceding formulas as subscripted variable i.

By way of example, four surface patch functions are highlighted in FIG. 7 as the subserosal wall location 72. The i and j subscripts mentioned previously correspond to indices for the lines of latitude and longitude of the bladder surface. For the purposes of this discussion, i will correspond to lines of longitude and j will correspond to lines of latitude although it should be noted the meanings of i and j can be interchanged with a mathematically equivalent result. Using the scanplane and integration line definitions provided in FIG. 7, the four surface patch functions are identified, in the clockwise direction starting in the upper left, as s_{36,62}, s_{40,62}, s_{40,64}, and s_{36,64}.

The surface patches are defined as functions of the patch coordinates, s_{i,j}(u,v). The patch coordinates u and v, are defined such that 0≦u, v<1 where 0 represents the starting latitude or longitude coordinate (the i and j locations), and 1 represents the next latitude or longitude coordinate (the i+1 and j+1 locations). The surface function could also be expressed in Cartesian coordinates where s_{i,j}(u,v)=x_{i,j}(u,v)i+y_{i,j}(u,v)j+z_{i,j}(u,v)k where i, j, k, are unit vectors in the x-, y-, and z-directions respectively. In vector form, the definition of a surface patch function is given in Equation 1.

$${s}_{i , j} ( u , v ) = [ \begin{matrix}{x}_{i , j} ( u , v ) \\ {y}_{i , j} ( u , v ) \\ {z}_{i , j} ( u , v )\end{matrix} ] . \qquad Equation 1$$

With the definitions of surface patch functions complete, attention can turn to the surface area calculation represented in the fifth block 54 of FIG. 6. The surface area of S, A(S), can be defined as the integration of an area element over the surface S, as shown in Equation 2. Since S is composed of a number of the patch surface functions, the calculation for the area of the surface S can be rewritten as the sum of the areas of the individual surface patch functions as in Equation 3.

$$\begin{matrix}A ( S ) = {\int }_{s} d A . & Equation 2 \\ A ( S ) = {\sum }_{i , j} A ( {s}_{i , j} ) . & Equation 3\end{matrix}$$

Similarly, to Equation 2 for the entire surface, the area of the surface patch is the integration of an area element over the surface patch, shown in Equation 4. The integration over the surface patch function can be simplified computationally by transforming the integration over the surface to a double integration over the patch coordinates u and v. The transformation between the surface integration and the patch coordinate integration is shown in Equation 5.

$$\begin{matrix}A ( {s}_{i , j} ) = {\int }_{{s}_{i , j}} d {A}_{i , j} . & Equation 4 \\ {\int }_{{s}_{i , j}} d {A}_{i , j} = {\int }_{u = 0}^{1} {\int }_{v = 0}^{1} \vert \frac{\partial {s}_{i , j}}{\partial u} \times \frac{\partial {s}_{i , j}}{\partial v} \vert d v d u . & Equation 5\end{matrix}$$

By substituting Equation 5 into Equation 4, and Equation 4 into Equation 3, the area for the entire surface can be calculated. The result of these substitutions is shown in Equation 6.

$$A ( S ) = {\sum }_{i , j} {\int }_{u} {\int }_{v} \vert \frac{\partial {s}_{i , j}}{\partial u} \times \frac{\partial {s}_{i , j}}{\partial v} \vert d v d u . \qquad Equation 6$$

The surface patch function may be any function that is continuous in its first derivatives. In the embodiment shown, a cubic B-spline interpolating function is used for the interpolating surface patch function although any surface function may be used. This interpolating function is applied to each of the Cartesian coordinate functions shown in Equation 1. The interpolating equation for the x-coordinate of the s_{i,j} patch function is given in Equation 7. Similar calculations are performed for the y_{i,j} and z_{i,j} components of the surface patch function.

x_{i,j}(u,v)=uM_{b}X_{i,j}M_{b}^{t}v^{t} Equation 7.

where t denotes matrix and vector transpose,

$$u = [ \begin{matrix}{u}^{3} \\ {u}^{2} \\ u \\ 1\end{matrix} ] , v = [ \begin{matrix}{v}^{2} \\ {v}^{2} \\ v \\ 1\end{matrix} ] , {M}_{b} = [ \begin{matrix}- 1 & 3 & - 3 & 1 \\ 3 & - 6 & 3 & 0 \\ - 3 & 0 & 3 & 0 \\ 1 & 4 & 1 & 0\end{matrix} ] , and$$

$${X}_{i , j} = [ \begin{matrix}{x}_{i - 1 , j - 1} & {x}_{i - 1 , j} & {x}_{i - 1 , j + 1} & {x}_{i - 1 , j + 2} \\ {x}_{i , j - 1} & {x}_{i , j} & {x}_{i , j + 1} & {x}_{i , j + 2} \\ {x}_{i + 1 , j - 1} & {x}_{i + 1 , j} & {x}_{i + 1 , j + 1} & {x}_{i + 1 , j + 2} \\ {x}_{i + 2 , j - 1} & {x}_{i + 2 , j} & {x}_{i + 2 , j + 1} & {x}_{i + 2 , j + 2}\end{matrix} ]$$

Since the interpolating functions for each of the patch functions is a cubic surface, the integration may be performed exactly using a quadrature formula. The formula used in this application is shown in Equation 8.

$$A ( {s}_{i , j} ) = {\sum }_{i , j} \frac{1}{4} ( \begin{matrix}{\vert \frac{\partial {s}_{i , j}}{\partial u} \times \frac{\partial {s}_{i , j}}{\partial v} \vert}_{u = \frac{3 - \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 - \sqrt{3}}{6} , v = \frac{3 + \sqrt{3}}{6}} + \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 - \sqrt{3}}{6}} + \\ {\vert \frac{\partial {s}_{i , j}}{\partial u} \times \frac{\partial {s}_{i , j}}{\partial v} \vert}_{u = \frac{3 + \sqrt{3}}{6} , v = \frac{3 + \sqrt{3}}{6}}\end{matrix} ) . \qquad Equation 8$$

Recalling the fact that s_{i,j}(u,v) is defined as a vector function in Cartesian coordinates (Equation 1), the norm of the cross product of the partial derivatives can be written as follows:

$$\vert \frac{\partial {s}_{i , j}}{\partial u} \times \frac{\partial {s}_{i , j}}{\partial u} \vert = \sqrt{\begin{matrix}{( \frac{\partial {y}_{i , j}}{\partial u} \frac{\partial {z}_{i , j}}{\partial v} - \frac{\partial {z}_{i , j}}{\partial u} \frac{\partial {y}_{i , j}}{\partial v} )}^{2} + \\ {( \frac{\partial {z}_{i , j}}{\partial u} \frac{\partial {x}_{i , j}}{\partial v} - \frac{\partial {z}_{i , j}}{\partial u} \frac{\partial {x}_{i , j}}{\partial v} )}^{2} + \\ {( \frac{\partial {x}_{i , j}}{\partial u} \frac{\partial {y}_{i , j}}{\partial v} - \frac{\partial {y}_{i , j}}{\partial u} \frac{\partial {x}_{i , j}}{\partial v} )}^{2} .\end{matrix}} \qquad Equation 9$$

When the physical x-, y-, and z-locations are used in the interpolating function, the surface are will be calculated in the square of the units of x, y, and z. At this point, the calculation in the fifth block 54 of FIG. 6 is complete.

Wall thickness determination. The second component to the mass calculation is a measurement of the thickness of the bladder muscle wall. This thickness is defined to be the normal thickness between the subserosal and submucosal surfaces of the bladder wall.

The wall thickness is calculated from the fractal dimension of the RF signal in the region of the wall thickness. The fractal dimension increases due to the multiplicity of interface reflections through the bladder muscle. The increase and decrease of fractal dimension through the bladder muscle wall can be modeled as a parabola where the fractal dimension is a function of the depth in the region of the bladder wall. The thickness of the bladder is then determined to be the region of the parabola model that is at least 97% of the maximal value of the fractal dimension. The calculations are reviewed below in Equation 10.

$${fd}_{r} = \frac{log ( \frac{\begin{matrix}max ( {RF}_{r = r - w / 2 , r + w / 2} ) - \\ min ( {RF}_{r = r - w / 2 , r + w / 2} ) + w\end{matrix}}{w} )}{log ( \frac{n}{w} )} . \qquad Equation 10$$

The fractal dimension calculation corresponds to the fourth block 56 of FIG. 6. The fractal dimension is calculated for a window of length w. In the current embodiment, the value of w is 5, the number of sample points along a scanline, although that value can be varied. The fractal dimension is calculated from the difference between the maximum RF signal value in the window centered at a given depth, r, and the minimum of that same window. The length of the window, w, is added to this difference, and the result is then normalized with the length of the window. The logarithm of that result is then divided by the logarithm of the ratio of the total number of samples in a scanline, n, to the length of the window. The calculation of the fractal dimension at each depth along a scanline is shown in Equation 10. This fractal dimension measure is calculated for the central n-w samples in a scanline.

After the measurements of the fractal dimension have been calculated based on the ultrasound signal, the thickness of the bladder wall may be calculated. The following calculations correspond to the seventh block 58 of FIG. 6.

The fractal dimension, fd, of the RF signal in the region of the bladder muscle wall is then modeled as a parabolic equation as a function of depth, r. The model of the equation for a single depth point is given in Equation 11. In that equation, there are 3 parameters (a, b, and c) that define the parabola with the depth along a scanline r, and the addition of a random element ε. The subscript i indicates a specific value of r, fd, and ε.

fdᵢ=arᵢ²+brᵢ+c+εᵢ Equation 11.

An equation of the form in Equation 11 is obtained for each depth point in the region of the wall. The number of observations is variable and depends on the thickness of the bladder wall as observed by the ultrasound signal. Assuming a set of n observations, the subscript i would count the observations from 1 to n. The set of n equations of the form in Equation 11 may be compressed into a matrix equation given in Equation 12. Each row of the fd, and ε, and the X matrix correspond to one of the n observations. The parabola parameters of Equation 11 are collected in the vector β.

$$fd = X β + ɛ \\ where fd = [ \begin{matrix}{fd}_{1} \\ {fd}_{2} \\ ⋮ \\ {fd}_{n}\end{matrix} ] , \\ X = [ \begin{matrix}{r}_{1}^{2} & {r}_{1} & 1 \\ {r}_{2}^{2} & {r}_{2} & 1 \\ ⋮ & ⋮ & ⋮ \\ {r}_{n}^{2} & {r}_{n} & 1\end{matrix} ] , β = [ \begin{matrix}a \\ b \\ c\end{matrix} ] , and ɛ = [ \begin{matrix}{ɛ}_{1} \\ {ɛ}_{2} \\ ⋮ \\ {ɛ}_{n}\end{matrix} ] . \qquad Equation 12$$

The next step is to estimate the values of the parameters of the parabola in the set of n equations of the form in Equation 11 or in the matrix Equation 12 based on the set of observations. A least-squares estimation of the parameters is used, and the calculation for these estimates is shown in Equation 13. In Equation 13, the t superscript indicates matrix transpose, and the −1 superscript indicates the matrix inverse. Parameters with hats (̂) indicate that the value is the least-squares estimate of those parameters.

\hat{β}=(X^{t}X)⁻¹X^{t}fd Equation 13.

The estimates of the parabola parameters (\hat{β}=└â \hat{b} ĉ┘^{t}) can be substituted into the parabola model to calculate the estimated fractal dimension at each depth r, as shown in Equation 14. The location of the maximum fractal dimension can be determined by setting the first derivative of the parabola model to equal 0 (Equation 15) and solving for r. The location where the fractal dimension is maximal is given in Equation 16.

$$\begin{matrix}f \hat{d} ( r ) = \hat{a} {r}^{2} + \hat{b} r + \hat{c} . & Equation 14 \\ \frac{d f \hat{d} ( r )}{d r} = 2 \hat{a} r + \hat{b} = 0. & Equation 15 \\ {r}_{{fd}_{max}} = - \frac{\hat{b}}{2 \hat{a}} . & Equation 16\end{matrix}$$

To determine the maximal fractal dimension as defined by the parabolic model, simply substitute Equation 16 into Equation 14 and solve for fdₘₐₓ. The resulting value is shown in Equation 17.

$$f {\hat{d}}_{max} = \frac{- {\hat{b}}^{2} + 4 \hat{c}}{4 \hat{a}} . \qquad Equation 17$$

To determine the locations where the fractal dimension is 97% of the maximum value, multiply Equation 17 by 0.97, substitute the result into Equation 14 and solve for r using the quadratic formula. The locations where the fractal dimension is 97% of the maximum value, r_{97%}, are given in Equation 18.

$${r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} . \qquad Equation 18$$

Two values for r_{97%} will be calculated from Equation 18. The difference between those two values will identify the thickness of the bladder muscle wall along the given scanline. Since these scanlines may or may not be perpendicular to the bladder muscle surface and bladder wall thickness must be measured along a line perpendicular to the bladder surface, a collection of these measurements are combined to determine the actual thickness of the bladder wall.

These measurements could be made at any surface of the bladder muscle wall. In FIG. 8, three scanlines (a first scanline 36, a second scanline 40, and a third scanline 44) are shown to cross the bladder muscle in two locations: the anterior wall closest to the transducer, and the posterior wall furthest from the transducer. The dotted portion of the lines represents the portion of the scanplanes that passes through the bladder muscle wall. The first 36, the second 40, and third 44 scanlines are shown transmitting through the subserosal wall location 72 and submucosal wall location 74. The parabolic model described previously can be applied twice on each to determine the thickness of both the anterior and posterior wall. The maximum and minimum and mean values of these thicknesses are used in the mass calculation and historical tracking of data. In the embodiment shown, this final thickness determination marks the end of the process identified in the seventh block 58 of FIG. 6.

In the preferred embodiment, the bladder is assumed to have a uniform wall thickness, so that a mean wall thickness value is derived from the scanned data and used for the bladder mass determination. Only three scanlines are shown in a plane, each separated by 1.5 degrees from each other. Both the number of scanlines in the plane and the angles separating each scanline within a plane may be varied.

Bladder mass determination. Once the thickness and the surface area have been measured, the mass of the bladder may be calculated. The volume of muscle tissue is assumed to be the surface area times the wall thickness, where the assumption is based on a uniform wall thickness at all points around the bladder. The mass is then the product of the volume of muscle tissue, the specific gravity of the bladder muscle tissue and the density of water. The specific gravity of bladder muscle is a known value readily available in medical reference texts. In the embodiment shown, this mass calculation corresponds to the eighth block 59 of FIG. 6.

In an alternate embodiment, the methods to obtain the wall-thickness data and the mass data via downloaded digital signals can be configured by the microprocessor system for remote operation via the Internet web-based system. The Internet web-based system (“System For Remote Evaluation Of Ultrasound Information Obtained By A Program Application-Specific Data Collection Device”) is described in co-pending and commonly assigned patent application Ser. No. 09/620,766, herein incorporated by reference. The internet web-based system has multiple programs that collect, analyze, and store organ thickness and organ mass determinations. These alternate embodiments thus provides an ability to measure the rate at which internal organs undergo hypertrophy with time and permits disease tracking, disease progression, and the provision of educational instructions to patients and caregivers.

FIG. 9 depicts a substantially bas-relief 2D presentation volume rendering of the left (first) and right (second) half bladder hemisphere views of a bladder. The first and second hemispheric views are virtual images that provides to the physician the similar look of a bladder as seen with an optical cystoscope and provides a non-invasive means to diagnose bladder-related programs using the volume renderings from image processing of digitized ultrasound echoes presented in the image cones. The image cones are either the 3D arrays of 2D scanplanes, or the 3D scan cone of 3D distributed scanlines. The image processing includes algorithms to normalize unbalanced intensity distributions along a scanline (General Software Time Gain Control), normalize ultrasound echo variation caused by differences in surface reflectivity (Reverberation Control), normalize ultrasound conduction differences between fluid regions and surrounding tissues (Underneath Fluid Compensation), and a 3D viewing software tool to present the substantially bas-relief 2D presentation. The image processing algorithms to obtain the bas-relief 2D presentation volume rendering is described in co-pending and commonly assigned provisional Patent Application Ser. No. 60/470,525 (“Ultrasound Virtual Cystoscope System and Method”), herein incorporated by reference. The left bladder hemisphere shows a bladder wall 304A and an artifact of ultrasound imaging, an acoustical shadow 308. Similarly, the right bladder hemisphere shows a bladder wall 304b, and a simulated bladder stone 312 artificially added to the data set.

The acoustical shadow 308 ultrasound artifact is put to good use by the system and method of the invention to foster the visualization of the simulated bladder stone 308. Near the acoustical shadow 308 is a set of low-resolution vertical lines that delineates the simulated bladder stone 312. The white arrowhead in FIG. 9 points to a single vertical line near the region underneath the simulated bladder stone 312 along the scanline where the region around the acoustical shadow 308 is imaged near the simulated bladder stone 312.

FIG. 1 is a side elevational view of an ultrasound transceiver 10. Transceiver 10 includes a transceiver housing 18 having an outwardly extending handle 12 suitably configured to allow a user to manipulate transceiver 10. The handle 12 includes a trigger 14 that allows the user to initiate an ultrasound scan of a selected anatomical portion, and a cavity selector 16, described below. Transceiver 10 includes a transceiver dome 20 that contacts a surface portion of the patient when the selected anatomical portion is scanned to provide an appropriate acoustical impedance match and to properly focus ultrasound energy as it is projected into the anatomical portion. The transceiver 10 further includes an array of separately excitable ultrasound transducer elements (not shown in FIG. 1) positioned within the housing 18. The transducer elements are suitably positioned within the housing 18 to project ultrasound energy outwardly from the dome 20, and to permit reception of acoustic reflections generated by internal structures within the anatomical portion. The array of ultrasound elements may include a one-dimensional, or a two-dimensional array of piezoelectric elements that are moved within the housing 18 by a motor, of a transceiver dome 20 that contacts a surface portion of the patient when the selected anatomical portion is scanned, or other similar actuation means to scan the selected anatomical region. Alternately, the array may be stationary with respect to the housing 18 so that the selected anatomical region is scanned by selectively energizing the elements in the array. Transceiver 10 includes a display 24 operable to view processed results from the ultrasound scan, and to allow operational interaction between the user and the transceiver 10. Display 24 may be configured to display alphanumeric data that indicates a proper and/or optimal position of the transceiver 10 relative to the selected anatomical portion. In other embodiments, two- or three-dimensional images of the selected anatomical region may be displayed on the display 24. The display 24 may be a liquid crystal display (LCD), a light emitting diode (LED) display, a cathode ray tube (CRT) display, or other suitable display devices operable to present alphanumeric data and/or graphical images to a user.

Still referring to FIG. 1, the cavity selector 16 is structured to adjustably control the transmission and reception of ultrasound signals to the anatomy of a patient. In particular, the cavity selector 16 adapts the transceiver 10 to accommodate various anatomical details of male and female patients. For example, when the cavity selector 16 is adjusted to accommodate a male patient, the transceiver 10 is suitably configured to locate a single cavity, such as a urinary bladder in the male patient. In contrast, when the cavity selector 16 is adjusted to accommodate a female patient, the transceiver 10 is configured to image an anatomical portion having multiple cavities, such as a bodily region that includes a bladder and a uterus. Alternate embodiments of the transceiver 10 may include a cavity selector 16 configured to select a single cavity scanning mode, or a multiple cavity-scanning mode that may be used with male and/or female patients. The cavity selector 16 may thus permit a single cavity region to be imaged, or a multiple cavity region, such as a region that includes a lung and a heart to be imaged.

To scan a selected anatomical portion of a patient, the transceiver dome 20 of the transceiver 10 is positioned against a surface portion of a patient that is proximate to the anatomical portion to be scanned. The user then actuates the transceiver 10 by depressing trigger 14. In response, transceiver 10 transmits ultrasound signals into the body, and receives corresponding return echo signals that are at least partially processed by the transceiver 10 to generate an ultrasound image of the selected anatomical portion. In a particular embodiment, the transceiver 10 transmits ultrasound signals in a range that extends from approximately about two megahertz (MHz) to approximately about ten MHz.

In one embodiment, the transceiver 10 is operably coupled to an ultrasound system that is configured to generate ultrasound energy at a predetermined frequency and/or pulse repetition rate and to transfer the ultrasound energy to the transceiver 10. The system also includes a processor that is configured to process reflected ultrasound energy that is received by the transceiver 10 to produce an image of the scanned anatomical region. Accordingly, the system generally includes a viewing device, such as a cathode ray tube (CRT), a liquid crystal display (LCD), a plasma display device, or other similar display devices, that may be used to view the generated image. The system may also include one or more peripheral devices that cooperatively assist the processor to control the operation of the transceiver 10, such a keyboard, a pointing device, or other similar devices. The ultrasound system will be described in greater detail below. In still another particular embodiment, the transceiver 10 may be a self-contained device that includes a microprocessor positioned within the housing 18 and software associated with the microprocessor to operably control the transceiver 10, and to process the reflected ultrasound energy to generate the ultrasound image. Accordingly, the display 24 is used to display the generated image and/or to view other information associated with the operation of the transceiver 10. For example, the information may include alphanumeric data that indicates a preferred position of the transceiver 10 prior to performing a series of scans. In yet another particular embodiment, the transceiver 10 may be operably coupled to a general-purpose computer, such as a laptop or a desktop computer that includes software that at least partially controls the operation of the transceiver 10, and also includes software to process information transferred from the transceiver 10, so that an image of the scanned anatomical region may be generated.

Although transceiver 10 of FIG. 1 may be used in any of the foregoing embodiments, other transceivers may also be used. For example, the transceiver may lack one or more features of the transceiver 10. For example, a suitable transceiver may not be a manually portable device, and/or may not have a top-mounted display, or may selectively lack other features or exhibit further differences.

FIG. 2 is an isometric view of an ultrasound scancone 30 that projects outwardly from the transceiver 10 of FIG. 1 that will be used to further describe the operation of the transceiver 10. The ultrasound scancone 30 extends outwardly from the dome 20 of the transceiver 10 and has a generally conical shape comprised of a plurality of discrete scanplanes having peripheral scanlines 31A-31F that define an outer surface of the scancone 30. The scanplanes also include internal scanlines 34A-34C that are distributed between the respective peripheral scanlines 31A-31F of each scanplane. The scanlines within each scanplane are one-dimensional ultrasound A-lines that taken as an aggregate define the conical shape of the scancone 30.

With reference still to FIG. 2 and now also to FIG. 3A, an ultrasound scancone 40 formed by a rotational array of two-dimensional scanplanes 42 projects outwardly from the dome 20 of the transceiver 10. The plurality of scanplanes 40 are oriented about an axis 11 extending through the transceiver 10. Each of the scanplanes 42 are positioned about the axis 11 at a predetermined angular position θ. The scanplanes 42 are mutually spaced apart by angles θ₁ and θ₂. Correspondingly, the scanlines within each of the scanplanes 42 are spaced apart by angles φ₁ and φ₂. Although the angles θ₁ and θ₂, are depicted as approximately equal, it is understood that the angles θ₁ and θ₂ may have different values. Similarly, although the angles φ₁ and φ₂ are shown as approximately equal, the angles φ₁ and φ₂ may also have different angles.

Referring now also to FIG. 3B, the peripheral scanlines 44 and 46, and an internal scanline 48 is further defined by a length r that extends outwardly from the transceiver 10 (FIG. 3A). Thus, a selected point P along the peripheral scanlines 44 and 46 and the internal scanline 48 may be defined with reference to the distance r and angular coordinate values φ and θ.

With continued reference to FIGS. 2, 3A and 3B, the plurality of peripheral scanlines 31A-E and the plurality of internal scanlines 34A-D are three-dimensional-distributed A-lines (scanlines) that are not necessarily confined within a scanplane, but instead may sweep throughout the internal regions and along the periphery of the scancone 30 (FIG. 2). Thus a given point P within the scancone 30 may be identified by the coordinates r, φ, and θ whose values can vary. The number and location of the internal scanlines emanating from the transceiver 10 may thus be distributed within the scancone 30 at different positional coordinates as required to sufficiently visualize structures or images within the scancone 30. As described above, the angular movement of the transducer may be mechanically effected, or it may be electronically generated. In either case, the number of lines and the length of the lines may vary, so that the tilt angle φ sweeps through angles approximately between −60° and +60° for a total arc of approximately 120°. In one embodiment, the transceiver 10 is configured to generate a plurality of scanlines between the first limiting scanline 44 and the second limiting scanline 46 of approximately about seventy-seven, each having a length of approximately about 18 to 20 centimeters (cm).

As previously described, the angular separation between adjacent scanlines 34 (FIG. 2) may be uniform or non-uniform. For example, and in another particular embodiment, the angular separation φ₁ and φ₂ (as shown in FIG. 2) may be about 1.5°. Alternately, and in another particular embodiment, the angular separation φ₁ and φ₂ may be a sequence wherein adjacent angles are ordered to include angles of 1.5°, 6.8°, 15.5°, 7.2°, and so on, where a 1.5° separation is between a first scanline and a second scanline, a 6.8° separation is between the second scanline and a third scanline, a 15.5° separation is between the third scanline and a fourth scanline, a 7.2° separation is between the fourth scanline and a fifth scanline, and so on. The angular separation between adjacent scanlines may also be a combination of uniform and non-uniform angular spacings, for example, a sequence of angles may be ordered to include 1.5°, 1.5°, 1.5°, 7.2°, 14.3°, 20.2°, 8.0°, 8.0°, 8.0°, 4.3°, 7.8°, so on.

After a scanplane 42 is generated, the transceiver 10 rotates the transducer through a rotational angle θ (FIG. 3A) to position the transducer assembly within the transceiver 10 to a different angular increment, to generate another scanplane. As the transducer assembly is rotated in the direction θ, a series of scanplanes is generated, with each scanplane slightly rotated in relation to the prior scanplane by a selected increment of the rotational angle θ. As previously described, the increment between adjacent scanplanes may be uniform or no uniform. For example, and with reference still to FIG. 3B, in another particular embodiment, each scanplane 42 may be projected at an approximately 7.5° rotational angle increment. In other embodiments, the angular increment may be non-uniform and arranged in a sequence wherein the spacing between adjacent scanplanes includes 3.0°, 18.5°, 10.2°, and so on. Accordingly, an increment of approximately 3.0° is present between a first scanplane and a second scanplane, an increment of approximately 18.5° is present between the second scanplane and a third scanplane, and an increment of approximately 10.2° is present between the third scanplane and a fourth scanplane, and so on. The scanplane interval may also be a combination of uniform and non-uniform rotational angle increments, such as, for example, a sequence of incremental angles ordered in a sequence including 3.0°, 3.0°, 3.0°, 18.5°, 10.2°, 20.6°, 7.5°, 7.5°, 7.5°, 16.0°, 5.8° and so on.

FIG. 3C is a scancone 40 that is generated by the transceiver 10. The scancone 40 includes a dome cutout 41 near an apex of the scancone 40 that is formed, at least in part, to the presence of the transceiver dome 20 (as shown in FIG. 1). Referring now to FIG. 3D, a plan view of the scancone 40 of FIG. 3D is shown. The dome cutout 41 is positioned at an approximate center of the scancone 40, with each of the scanplanes 42 mutually spaced apart by the angular increment θ. Although the scancone 40 includes forty-eight scanplanes 42 that are mutually uniformly spaced apart, the number of scanplanes 42 in the scancone 40 may include at least two, but can be varied to include any desired number of scanplanes 42.

FIG. 3E is side-elevational view of the scanplane 42 of FIG. 3C and FIG. 3D that includes approximately about seventy-seven scanlines 48 that extend outwardly from the dome cutout 41. Other scancone configurations are possible. For example, a wedge-shaped scancone, or other similar shapes may be generated by the transceiver 10 (FIG. 1).

FIG. 4A is an isometric view of the transceiver 10 of FIG. 1 applied to an abdominal region of a patient, which is representative of a data acquisition method for a bladder wall mass determination in the patient. In contact with the patient is a pad 67 containing a sonic coupling gel to minimize ultrasound attenuation between the patient and the transceiver 10. Alternatively, sonic coupling gel may be applied to the patient's skin. The dome 20 (not shown) of the transceiver 10 contacts the pad 67. The transceiver 10 may the used to image the bladder trans-abdominally, and initially during a targeting phase, the transceiver 10 is operated in a two-dimensional continuous acquisition mode. In the two-dimensional continuous mode, data is continuously acquired and presented as a scanplane image as previously shown and described. The data thus acquired may be viewed on a display device, such as the display 24, coupled to the transceiver 10 while an operator physically translates the transceiver 10 across the abdominal region of the patient. When it is desired to acquire data, the operator may acquire data by depressing the trigger 14 of the transceiver 10 to acquire real-time imaging that is presented to the operator on the display device.

FIG. 4B is a perspective view of the transceiver 10 of FIG. 1 positioned in a communication cradle 50 according to another embodiment of the invention. The communication cradle 50 is operable to receive the transceiver 10, and to transfer data and/or electrical energy to the transceiver 10. In another particular embodiment of the invention, the cradle 50 may include a data storage unit configured to receive imaging information generated by the transceiver 10 (not shown), and may also include a data interface 13 that may be employed to transfer the acquired imaging information to other processors or systems for further image processing. In a particular embodiment, the data interface may include a universal serial bus (USB) interface having a connecting cable 53. In other embodiments, the data interface 13 may include a FIREWIRE interface, an RS-232 interface, or other similar and known interface devices. In still another particular embodiment, the data interface 13 may be used to transfer programmed instructions to a processing device positioned within the transceiver 10.

FIG. 5 is a partially schematic view of an imaging system 51 according to another embodiment of the invention. The system 51 includes at least one transceiver 10 in communication with a computer device 52 that is further in communication with a server 56. The at least one transceiver 10 is operable to project ultrasound energy into a patient and to receive the resulting ultrasound echoes, as previously described. The ultrasound echoes may be converted to digital signals within the transceiver 10, or alternately within the computer device 52 that is coupled to the transceiver 10. Similarly, the digital signals may be stored and processed in the transceiver 10, or within the computer device 52 to generate ultrasound images that may be viewed on a display 54 that is coupled to the computer device 52. In either case, the transceiver 10 may be coupled to the computer device 52 by the connecting cable 53, or by means of a wireless link, such as an ETHERNET link, or an infrared wireless link. The transceiver 10 and/or the computer device 52 are configured to process the digital signals using algorithms that will be explained in greater detail below.

Still referring to FIG. 5, the computer device 52 may communicate information to the server 56, which is configured to receive processed images and/or image data from the computer device 52 and/or the transceiver 10. The server 56 may include any computer software and/or hardware device that is responsive to requests and/or issues commands to or from at least one client computer (not shown in FIG. 5). The server 56 is coupled to the computer device 52 by a local communications system 55, such as a telephone network or a local area network (LAN) or other similar networks.

The operation of the imaging system 51. Each transceiver 10 may be separately and independently used to project ultrasound information into a selected region of the patient and to transmit the signals proportional to the received ultrasound echoes to the computer device 52 for storage and/or further processing. If the image processing occurs in the computer device 52, each computer device 52 includes imaging software having instructions to prepare and analyze a plurality of one dimensional images from the stored signals and to transform the plurality of images into a plurality of two-dimensional scanplanes, as previously described. Additionally, the imaging software programs may also present three-dimensional renderings from the plurality of two-dimensional scanplanes. Each computer device 52 may also include instructions to perform other additional ultrasound image enhancement procedures, which may include instructions to implement the image processing algorithms.

In another embodiment of the system 51, the imaging software programs and other instructions that perform additional ultrasound enhancement procedures are located on the server 56. Each computer device 52 coupled to the system 51 receives the acquired signals from the transceiver 10 using the cradle 50 and stores the signals in the memory of the computer device 52. The computer device 52 subsequently retrieves the imaging software programs and the instructions to perform the additional ultrasound enhancement procedures from the server 56. Thereafter, each computer device 52 prepares the one-dimensional images, the two-dimensional images, and the three-dimensional renderings, as well as enhanced images from the retrieved imaging and ultrasound enhancement procedures. Results from the data analysis procedures may then be sent to the server 56 for storage.

In still another embodiment of the system 51, the imaging software programs and the instructions to perform the additional ultrasound enhancement procedures are located in the server 56 and executed on the server 56. Each computer device 52 in the system 51 receives the acquired signals from the transceiver 10 and sends the acquired signals to the memory of the computer 52 through the cradle 50. The computer device 52 subsequently sends the stored signals to the server 56. In the server 56, the imaging software programs and the instructions to perform the additional ultrasound enhancement procedures are executed to prepare the one-dimensional images, two-dimensional images, three-dimensional renderings, and enhanced images from the signals. Results from the data analysis procedures may be stored by the server 56, or alternatively, sent to a client computer coupled to the server for archival storage, or for other purposes.

FIG. 6 is a partially schematic view of a networked imaging system 61 according to still another embodiment of the invention. Many of the elements of the present embodiment have been discussed in detail in connection with other embodiments, and in the interest of brevity, will not be discussed further. The networked imaging system 61 includes a public data network 64 interposed between the computer device 52 and the server 66. The public data network 64 may include a LAN, a WAN, or the Internet. Accordingly, other computational devices associated with the public data network 64 may communicate imaging data and/or ultrasound images with the portable computing devices 52 and the server 56. Although two transceivers 10 are shown in the networked imaging system 61 shown in FIG. 6, fewer that two, or more than two transceivers 10 may be present. The public data network 64 advantageously permits the system 61 to communicate images and data to other computer devices and/or processors.

FIG. 7 is a cross sectional view of a selected anatomical portion that will be used to further describe the various embodiments of the present invention. As shown in FIG. 7, the transceiver 10 is placed over the anatomical portion, which may include a urinary bladder and surrounding tissues of a male patient. Also shown, the dome 20 of the transceiver is placed in contact with a sonic coupling gel contained within a pad 67 to minimize ultrasound attenuation between the patient and the transceiver 10. Alternatively, the dome 20 may be placed in contact with a sonic coupling gel applied on the patient's skin. A wall of the urinary bladder may be divided into three distinct and observable layers, including an outer wall layer (visceral peritoneum), an opposing inner wall layer, and an inter-wall layer positioned between the outer layer and the inner layer. In general, muscular contraction in the bladder results from muscular tissue in the inter-wall layer, so that urine within the bladder may be excreted. The bladder wall thickness typically varies between about 1.0 millimeter (mm) and about 4.0 millimeters (mm). Since the volume of the bladder wall is a product of an area of the bladder and the thickness of the bladder wall, an estimation of the bladder wall volume is reasonably accurate if the surface area determination of the bladder wall and the thickness of the bladder wall is sufficiently precise. Assuming the thickness of the bladder wall is substantially uniform around the bladder, a bladder wall mass can be calculated as a product of the bladder wall volume and an estimation of the density of the wall tissue. The bladder wall mass calculations are thus similarly limited by the accuracy of the bladder wall surface area determination and the bladder wall thickness measurement.

FIG. 8 is a cross sectional view of the anatomical region of FIG. 7 as the region is imaged by the transceiver 10. As previously described, the transceiver 10 is operable to image the anatomical region by generating a scanplane 42 that is further comprised of a plurality of scanlines 48. In FIG. 8, the partial scanplane 42 is superimposed on a B-mode ultrasound image of the anatomical region in order to illustrate the plurality of scanlines 48 crossing the front bladder wall (e.g., the wall closer to the dome 20) and extending through the bladder to the back wall of the bladder.

FIGS. 9A through 9D are four exemplary and sequential ultrasound images obtained from a male subject during an ultrasound examination. The ultrasound images were obtained using lower resolving B-mode algorithms, and show a bladder volume surrounded by a bladder wall. In FIGS. 9A through 9D, the front and back walls of the bladder are shown surrounding a generally darker bladder volume. As shown in FIGS. 9A through 9D, the front wall and the back wall of the bladder are relatively poorly defined.

FIGS. 10A through 10D are four exemplary and sequential ultrasound images obtained from a female subject during an ultrasound examination. The ultrasound images in FIGS. 10A through 10D were also obtained using lower resolving B-mode algorithms. In FIGS. 10A through 10D, the bladder is similarly poorly defined, and a uterine structure is detected beyond the bladder. The bladder front wall (BFW) and an opposing bladder back wall (BBW) along with the uterine front wall (UFW) and a uterine back wall (UBW) are imaged, but are still rather poorly defined. Thus, the ability to easily discern the front and back walls of a uterus and a bladder from the same female subject using selected wall locations obtained from B-mode imaging is difficult to establish. In particular, the determination of the narrower distances between the outer and inner wall layer locations of the uterus or bladder is often very difficult to establish.

FIG. 11 is an exemplary, non-rectified echogenic signal received along a selected scanline during ultrasound imaging of a bladder. The echogenic signal pattern includes an outer wall reflection, which is shown as a solid line, which results from a reflection that occurs at the outer wall of a bladder (as best seen in FIG. 7), and an inner wall reflection (as also shown in FIG. 7), resulting from a reflection occurring at an inner wall of the bladder, which is shown as a dashed line. Since the non-rectified inner wall reflection and the outer wall reflection signals at least partially overlap, it may be difficult to accurately discern a location of the inner wall of the bladder from the outer wall location.

FIG. 12 is an exemplary processed echo signal pattern from the selected scanline of the bladder imaging of FIG. 8. The outer wall and inner wall reflection signals are algebraically summed and rectified to generate a signal envelope waveform. Rectification is achieved by performing a Hilbert transform to the algebraically summed waveform. The positive signal envelope waveform obtained by the Hilbert transform advantageously allows a central location of the outer and the inner layers of the front organ walls to be accurately located since the envelope exhibits a more pronounced signal peak corresponding to the outer and the inner walls.

FIG. 13 is the processed echogenic signal pattern of FIG. 12 that further shows a waveform that is generated by additional processing of the rectified waveform. The waveform (represented by a dotted line in FIG. 13) may be generated by processing the rectified waveform of FIG. 12 using an A-mode algorithm so that selected bladder wall locations may be more easily identified. The processed rectified waveform is generally sharper and/or exhibits peaks that permit various maximum points on the processed rectified waveform may be easily identified. Once identified, the maximum waveform points may then be used to select candidate points for further bladder wall imaging, described below.

FIG. 14 is a method algorithm of the particular embodiments. The method algorithm 170 is comprised of 8 sub-algorithms that culminate in the calculation of the mass of the organ wall. In block 172, the ultrasound probe is positioned over the abdomen of a patient and a scan is commenced to acquire at least a portion of an organ wall image. The echoes are received and processed in the next block, block 176. A block 176 signals are generated from the echoes in proportion to their signal strength and the signals are processed and presented as a 2-D ultrasound image in the format of two-dimensional scanplanes. This is commonly referred to as B-mode ultrasound. The next block is block 180 in which the desired organ in the 2-D scanplanes is selected and wall loci of the organ wall in at least one scanplane is delineated. Algorithm 170 continues with block 184 in which the initial wall delineation from the 2-D scanplane is now further refined or adjusted. The adjustment of the 2-D wall loci position is achieved by applying a 1-D analysis of the scanline echo signals to obtain inner and outer wall layer loci. The next block is block 188 in which the thickness of the organ wall is calculated as a difference between the inner and outer wall layer loci as determined from block 184. The algorithm 170 continues with block 192 in which 2-D scanplanes obtained from B-mode ultrasound are assembled into a 3D array and the wall surface area of the organ wall is calculated. In block 300, the volume of the organ wall is calculated as a product of the thickness as determined from block 188 and the surface area as determined from block 192. Finally, in block 400, the mass of the organ wall is calculated as a product of the volume obtained from block 300 and the tissue density of the organ wall.

FIG. 15 is a flowchart that will be used to describe a method 170 for scanning a bodily organ, according to an embodiment. At block 172, ultrasound energy is projected into the bodily organ, and reflections from various internal structures are acquired, that constitutes raw ultrasound data. The raw data may be collected, for example, using the device shown in FIG. 1, or in any of the other disclosed embodiments described herein. Block 184 describes the procedures to obtain points for the inner and outer wall layers. At block 184A, the raw data is processed to generate an RF envelope, as earlier described and shown in FIG. 16 and FIG. 17. In addition, at block 176, B-mode scans of the bodily organ are also compiled. Based upon the B-mode data acquired at block 176, a family of bladder wall locations may be generated, as will be described below.

At block 184C, incident angles for each of the scanlines projected into the bodily organ are calculated as will also be described below. In general terms, the calculation of the incident angle permits better discrimination between an inner wall and an outer wall of the organ. At block 184E, candidate points that characterize the position of the inner wall, the outer wall and the position of intermediate layers between the inner wall and the outer wall are determined. The determination of candidate points will also be described in greater detail below. Based upon the candidate points generated at block 184E, candidate walls may be generated at block 184G. The candidate walls comprise a family of possible wall locations, which will be further processed, as described below.

Still referring to FIG. 15, at block 184J, an inner wall layer location is identified from amongst the candidate walls determined at block 184G. An outer wall location is also identified at block 184L, which represents a refined estimate of an actual outer wall layer location. The determination of the inner wall location and the outer wall location will be described in greater detail below. Based upon the inner wall layer and the outer wall layer determinations at blocks 184J and 184L, respectively, and the incident angle determinations at block 184C, a wall thickness may be determined at block 188. A surface area of the bodily organ may be determined based upon the B-mode data collected at block 192. Based upon the surface area determination at block 192 and the wall thickness determination at block 188, an organ volume value 300 and an organ mass value 400 for the organ wall may then be determined by routine calculation.

In FIG. 16, a method for determining incident angles will now be described. The scancone 20 of the transceiver 10 (FIG. 1) projects ultrasound energy towards an anatomical portion that includes a front wall of a bodily organ, such as a urinary bladder. In general, the scancone 20 is positioned at an angle Ω with respect to a normal direction relative to the bladder wall of a patient. A wall thickness is defined by a distance between an inner wall and an outer wall of the bladder along the surface normal T. Also, the inner and outer walls are most clearly discerned on scanlines that are normal to the bladder surface. Accordingly, the incident angle of each of the scanlines 48 of the scanplane 42 is supplied. A first vector R₁ extends along a first scanline having a tilt angle φ₁ and a second vector R₂ extends along a second scanline having a tilt angle φ₂. Accordingly, a vector R₁₂ that is a difference between the first vector R₁ and the second vector R₂ extends between R₁ and R₂.

In general, the vector \vec{R} extends from the cone vertex at an incident angle φ. In the interest of clarity of illustration, a two-dimensional representation of \vec{R} is shown in FIG. 18. It is understood, however, that the vector \vec{R} is oriented in three-dimensional space. Accordingly, in the description that follows, the vector \vec{R} may be expressed in equation E1 as:

\vec{R}=(R cos φ, R sin φ, 0) E1

where, R is the distance between the cone vertex and a segmentation point positioned on the front wall. The two adjacent neighbor points, \vec{R}₁ and \vec{R}₂, are expressed similarly in equation E2 and E3:

\vec{R}₁=(R₁ cos φ₁, R₁ sin φ₁, 0) E2

\vec{R}₂=(R₂ cos φ₂, R₂ sin φ₂, 0) E3

The surface vector, \vec{R}₁₂, may be expressed in terms of the two adjacent points, \vec{R}₁ and \vec{R}₂ by a vector addition, as follows in equation E4:

\vec{R}₁₂=\vec{R}₂−\vec{R}₁ E4

The surface normal vector T is orthogonal to the surface vector, \vec{R}₁₂ When the vector T is rotated through an angle θ about the y-axis, a rotation matrix and an orthogonal matrix may be defined, respectively, as follows:

$$[ \begin{matrix}cos {θ}^{′} & 0 & - sin {θ}^{′} \\ 0 & 1 & 0 \\ sin {θ}^{′} & 0 & cos {θ}^{′}\end{matrix} ] and [ \begin{matrix}0 & - 1 & 0 \\ 1 & 0 & 0 \\ 0 & 0 & 1\end{matrix} ]$$

where in the present case, θ′ is an angle between the orthogonal plane, and if the image is in a first plane, the angle θ′ will be zero, and if the image is the 13th plane (in a 24-plane image), the angle θ′ will be the incident angle of the broadside scanline relative to the first plane.

Therefore, a surface normal vector, \vec{R}₁₂^{⊥ccw} may be calculated using the above rotation and orthogonal matrices as described in equation E5.

$${\vec{R}}_{12}^{⊥ ccw} = [ \begin{matrix}cos {θ}^{′} & 0 & - sin {θ}^{′} \\ 0 & 1 & 0 \\ sin {θ}^{′} & 0 & cos {θ}^{′}\end{matrix} ] [ \begin{matrix}0 & - 1 & 0 \\ 1 & 0 & 0 \\ 0 & 0 & 1\end{matrix} ] {\vec{R}}_{12} \qquad E5$$

The angle between the two vectors, \vec{R} and \vec{R}₁₂^{⊥ccw} is the incident angle θ, which may be determined as follows in equation E6:

$$\begin{matrix}θ = \vec{R} ∠ {\vec{R}}_{12}^{⊥ ccw} = {cos}^{- 1} ( \frac{\vec{R} \cdot {\vec{R}}_{12}^{⊥ ccw}}{‖ \vec{R} ‖ \cdot ‖ {\vec{R}}_{12}^{⊥ ccw} ‖} ) & E6 \\ f ( x ) = {ax}^{2} + bx + c & E 9\end{matrix}$$

where “∥•∥” indicates a vector length and “•” is the dot product of the two vectors.

The above method can be extended to calculate the incidence angle in a three-dimensional space. In case of such a three dimensional extension, a two-dimensional plane is fit to all points in the neighborhood of point \vec{R}. The normal direction to this plane is determined \vec{R}₁₂^{⊥ccw} and then the incidence angle is calculated as in Equation E6. To fit the plane to a neighborhood of points and determine the normal direction to the plane, an eigenvector-based approach is used. First calculate a 3 by 3 covariance matrix C for all the points in the neighborhood of point \vec{R}. The eigenvalues and the eigenvectors of this 3 by 3 matrix are then calculated. Thereafter, the normal direction is determined the eigenvector corresponding the smallest eigenvalue.

FIG. 17 is a diagram that shows an idealized envelope having echogenic intensity distributed along a scanline similar to the scanline 48 of FIG. 8 that crosses the front bladder wall. In FIG. 17, only the echogenic pattern of the front bladder wall is shown, so that the strongly echogenic patterns caused by adipose and peritoneum tissues are not shown. The front wall profile shown in FIG. 17 is bimodal, and where the proximal wall outer layer generates an outer layer peak having a signal midpoint maxima near a distance value of 30, a middle layer (bladder muscle) having a signal minima near a distance value of 50, and a distal inner layer peak that presents another signal midpoint maxima near a distance value of 70. A search region for candidate points may therefore include at least the distance between the exterior slopes the outer and inner layers peaks, indicated by the vertical lines that intersect near a distance value of 25 for the outer layer peak and near a distance value of 75 for the inner layer peak.

FIG. 18 is an actual echogenic envelope distribution along a scanline that crosses highly reflective adipose and peritoneum tissues. The echogenic distribution is therefore more complex than the distribution shown in FIG. 17, since signal variation and/or noise are included. FIG. 18 also shows a plurality of possible candidate points that may be used to identify the inner and the outer wall layers of the bladder. The inner and outer wall layer candidate points are present as local peak maxima, and are shown by ovals in FIG. 18. The candidate points are determined by one-dimensional A-mode algorithms applied to the distribution, as will be discussed in more detail below. Accordingly, FIG. 18 shows, for example, a total of fifteen local maxima, which correspond to fifteen inner and outer layer candidate points, although either more than fifteen, or fewer than fifteen candidate points may be present in other similar distributions.

Still referring to FIG. 18, the inner and outer wall layer candidate points are developed by higher resolution one-dimensional algorithms applied to scanlines 48, (FIG. 8) which use an initial inner layer anchor point determined by a two-dimensional segmentation algorithm having generally less resolution. The initial inner layer anchor point on the scanline 48, which in the present example are determined by the two-dimensional B-mode segmentation algorithms, are shown in FIG. 18 as a diamond with dashed lines. The segmentation anchor point serves as a reference point that permits the adequacy of the one-dimensional inner and outer wall layer candidate points to be determined.

With continued reference to FIG. 18, localized peaks P1, P2, P3, and P4 are shown that resemble the outer-inner layer bimodal pattern of FIG. 17. For example, in the region between a distance 65 and a distance 71, the peaks P2 and P3 appear to closely approximate the bimodal pattern of FIG. 17 since the signal magnitude of the point P2 is approximately the same as for the point P3. A local minimum is present between the points P2 and P3, which correspond to two minor maxima. If the region between and including P2 and P3 represents a front bladder wall, then the higher magnitude P1 could be indicative of the more reflective peritoneal or adipose tissues that are anterior or proximate to the dome 20 (FIGS. 7 or 8).

Although the combination of the candidate points P2 and P3 appear to present a favorable candidate for the location of the outer and inner bladder walls, respectively, other combinations are possible. For example, the points P1 and P2, and the points P3 and P4 may also represent the location of the outer and inner bladder walls. Moreover, any combination of the fifteen local maxima or candidate points shown in FIG. 20 may be used to determine a location of the front wall. Algorithms will be described below that may be implemented to select envelope peak candidates within a particular scanline 48 with enhanced confidence. Accordingly, a peak combination representing the location of the bladder wall may be identified with increased accuracy.

FIG. 19 is a B-mode ultrasound image that shows a family of wall layer locations corresponding to the candidate points of FIG. 18 assembled from adjacent scanlines 48. The continuous white line shown in FIG. 19 represents an initial inner wall location of the bladder superimposed onto the image as determined by the two-dimensional B-mode segmentation algorithms. The dashed lines shown in FIG. 19 represent candidates for the location of outer wall layers that, in the present case, progress outwardly towards the dome cutout 41 (FIGS. 3C through 3E). The family of seven dashed lines indicate the seven possible outer layer wall locations, some of which are overlapping with the initial inner wall as determined by the two-dimensional B-mode segmentation algorithms.

As shown in FIG. 19, the application of all the candidate points (FIG. 18) suggests that estimates of the thickness of the bladder wall can vary from nearly zero, to multiple centimeters. Algorithms to identify an optimum set of candidate points from the group of all of the candidate points generated is therefore preferable to select the final wall locations so that a bladder wall thickness within an expected range is determined. In general, an expected range of bladder wall thicknesses is between approximately about one millimeter and about four millimeters. Accordingly, a search range from about −2 millimeters and about 10 millimeters may be used to search for candidate points on scanlines having large incident angles from the initial front inner wall location. The search range can also be determined based on the volume of urine in the bladder. For a given volume assuming a spherical bladder, we can calculate the minimum and the maximum expected wall thickness based on smallest and largest expected bladder masses. A smallest expected bladder mass value may be around 10 grams while a largest expected bladder mass value may be around 100 grams. Candidate points so identified may be defined as inner layer and outer layer candidate points.

FIG. 20 is a diagrammatic view of a plurality of candidate wall points that result from an echogenic distribution, such as the distribution shown in FIG. 19. In FIG. 20, for example, twenty-five wall envelope maxima are identified as candidate points in a relevant portion of the scanplane 42 (FIGS. 8, 15) selected from a series of truncated scanlines 48A through 48K from FIG. 19 that are selected from the scanplane 42. The total number of candidate points may be determined by a candidate points algorithm according to an embodiment of the invention, which will be described in further detail below. The wall layer locations are determined from the segmented front wall (FIG. 20) and the incident angle Ω of a selected scanline 48 (FIG. 15). As shown in FIG. 15, the wall thickness is defined along a surface normal extending outwardly from the front wall of the bladder wall. Alternate embodiments of the methods described for FIG. 16 permits the determination of organ wall thicknesses from non-normal incidence angles.

Of the wall candidate points shown in FIG. 20, nine of the candidate points are determined by the algorithms below to properly characterize a location of the nearest outer layer. The foregoing candidate points are shown in FIG. 20 as lightly shaded circles, while the remaining points, shown as dark circles, are retained as candidate points for an inner layer location determination. As shown in FIG. 20, the nine selected candidate points closely correlate with a candidate outer wall layer. An outer wall selection method algorithm identifies and selects the outer layer points from the plurality of scanlines 48A through 48K. The algorithm reduces the total number of candidate points while preserving appropriate candidate points.

FIG. 21A is a flowchart that will now be used to describe a method 220 for identifying an outer wall location based upon the candidate points, according to an embodiment of the invention. As an initial matter, all candidate points are selected for the analysis described below. In block 222, the outer wall location is first assumed to be at least 0.78 millimeters (mm) away from the inner wall, so that an initial wall thickness is at least about 0.78 mm. Accordingly, the equivalent sample distance is about 0.8 mm (about 20 RF sample points). At block 224, for each of the scanlines 48A through 48K (see FIG. 20), at least one upper most candidate point is selected for each of the respective scanlines 48A through 48K. In one particular embodiment, at least four uppermost candidate points are selected, and characterize the outer wall location, an inside wall location, and a muscular membrane positioned between the outer wall location and the inner wall location. At block 226, the selected candidate points are tested for consecutiveness. Any of the selected candidate points that are more than a predetermined distance away from an assumed inner wall location are rejected. In another particular embodiment, any point candidate point that is more than about 1.2 mm (about 30 RF sample points) away from the assumed inner wall location is discarded. At block 228, of the remaining candidate points, any candidate point having an intensity that is less than about one-half of the intensity among the selected candidates are also rejected. The foregoing blocks in the method are performed for incident angles greater than about 0.2 radian (about 10 degrees). Once the candidate points for the outer wall location have been selected, at block 230, a cost function is employed in order correlate an outer wall location with the candidate points. The cost function is based on the least-square error between the candidate wall locations and the candidate points. The candidate walls are calculated from the known incident angles by varying the wall thickness from 0 to about 78.4 mm. The cost function, Ci, is calculated by the following expression of equation E8:

$${C}_{i} = {( \frac{1}{n} {\sum }_{k = 1}^{n} \sqrt{min ( \vert {W}_{k} - C \vert )} )}^{2} \qquad E 8$$

Where n is the number of scanlines, Wₖ is the candidate wall location, and C are the candidate points. An exemplary cost function distribution that characterizes an outer wall location is shown in FIG. 19. Accordingly, an outer wall location is selected by identifying a minimum point in the distribution.

With reference now to FIG. 21B, a flowchart of a method 240 for identifying an inner wall location is shown, in accordance with another embodiment of the invention. At block 242, an inner wall range is restricted to fall within a predetermined range with respect to the outer wall location. In a particular embodiment of the invention, the predetermined range is between approximately about −0.4 mm and approximately about 1.0 mm relative to the outer wall location. At block 244, the intensity of a candidate point is assessed, and if the intensity of the candidate points are greater than approximately about one half of the intensity of a candidate point having a maximum intensity in the inner wall zone, the candidate points are retained. At block 246, if the intensity of a candidate point is less than that of any of the candidate points selected in block 244. During the foregoing inner wall selection, the process is performed only if the incident angle is greater than about 0.2 radian (about 10 degrees). The inner wall location is then selected by reverting to block 248, so that a minimum in cost function distribution may be determined.

Due to acoustic reverberation of the transceiver dome 20 and to additional noise introduced through segmentation, the front wall segmentation of a bodily organ, such as a bladder, may be unacceptable as a thickness measurement estimation. Accordingly, it has been determined that a well-defined wall segmentation may be fitted using a second order polynomial, although other higher order polynomials may be used. The second order polynomial least squares curve fitting will now be described. The segmented points, yᵢ, are known and the second degree polynomial, f(x) is expressed in equation E9 as:

The least-square error, Π, may be expressed by equation E10:

$$Π = {\sum }_{i = 1}^{n} {[ {y}_{i} - f ( {x}_{i} ) ]}^{2} = {\sum }_{i = 1}^{n} {[ {y}_{i} - ( {ax}_{i}^{2} + {bx}_{i} + c ) ]}^{2} \qquad E 10$$

Π is therefore minimized by varying the coefficient a, b, and c. Consequently, each of the partial derivatives of Π with respect to each coefficient is set to zero, as shown below in equation E11-13:

$$\begin{matrix}\frac{\partial Π}{\partial a} = 2 {\sum }_{i = 1}^{n} {x}_{i}^{2} [ {y}_{i} - ( {ax}_{i}^{2} + {bx}_{i} + c ) ] = 0 & E 11 \\ \frac{\partial Π}{\partial b} = 2 {\sum }_{i = 1}^{n} {x}_{i} [ {y}_{i} - ( {ax}_{i}^{2} + {bx}_{i} + c ) ] = 0 & E 12 \\ \frac{\partial Π}{\partial c} = 2 {\sum }_{i = 1}^{n} [ {y}_{i} - ( {ax}_{i}^{2} + {bx}_{i} + c ) ] = 0 & E 13\end{matrix}$$

Expanding the above equations, the following expressions are obtained as shown in equation E14-E16:

$$\begin{matrix}{\sum }_{i = 1}^{n} {x}_{i}^{2} {y}_{i} = a {\sum }_{i = 1}^{n} {x}_{i}^{4} + b {\sum }_{i = 1}^{n} {x}_{i}^{3} + c {\sum }_{i = 1}^{n} {x}_{i}^{2} & E 14 \\ {\sum }_{i = 1}^{n} {x}_{i} {y}_{i} = a {\sum }_{i = 1}^{n} {x}_{i}^{3} + b {\sum }_{i = 1}^{n} {x}_{i}^{2} + c {\sum }_{i = 1}^{n} {x}_{i} & E 15 \\ {\sum }_{i = 1}^{n} {y}_{i} = a {\sum }_{i = 1}^{n} {x}_{i}^{2} + b {\sum }_{i = 1}^{n} {x}_{i} + c {\sum }_{i = 1}^{n} 1 & E 16\end{matrix}$$

Expressing the foregoing in matrix form, the following matrix equation is obtained in equation E17:

$$[ \begin{matrix}{\sum }_{i = 1}^{n} {x}_{i}^{2} {y}_{i} \\ {\sum }_{i = 1}^{n} {x}_{i} {y}_{i} \\ {\sum }_{i = 1}^{n} {y}_{i}\end{matrix} ] = [ \begin{matrix}{\sum }_{i = 1}^{n} {x}_{i}^{4} & {\sum }_{i = 1}^{n} {x}_{i}^{3} & {\sum }_{i = 1}^{n} {x}_{i}^{2} \\ {\sum }_{i = 1}^{n} {x}_{i}^{3} & {\sum }_{i = 1}^{n} {x}_{i}^{2} & {\sum }_{i = 1}^{n} {x}_{i} \\ {\sum }_{i = 1}^{n} {x}_{i}^{2} & {\sum }_{i = 1}^{n} {x}_{i} & {\sum }_{i = 1}^{n} 1\end{matrix} ] [ \begin{matrix}a \\ b \\ c\end{matrix} ] \qquad E 17$$

Therefore, the coefficients a, b, and c for the least squares analysis may be determined as shown in equation E18:

$$[ \begin{matrix}a \\ b \\ c\end{matrix} ] = {[ \begin{matrix}{\sum }_{i = 1}^{n} {x}_{i}^{4} & {\sum }_{i = 1}^{n} {x}_{i}^{3} & {\sum }_{i = 1}^{n} {x}_{i}^{2} \\ {\sum }_{i = 1}^{n} {x}_{i}^{3} & {\sum }_{i = 1}^{n} {x}_{i}^{2} & {\sum }_{i = 1}^{n} {x}_{i} \\ {\sum }_{i = 1}^{n} {x}_{i}^{2} & {\sum }_{i = 1}^{n} {x}_{i} & {\sum }_{i = 1}^{n} 1\end{matrix} ]}^{- 1} [ \begin{matrix}{\sum }_{i = 1}^{n} {x}_{i}^{2} {y}_{i} \\ {\sum }_{i = 1}^{n} {x}_{i} {y}_{i} \\ {\sum }_{i = 1}^{n} {y}_{i}\end{matrix} ] \qquad E 18$$

If the least-square error between the wall segmentation and the second order polynomial is greater than about five pixels it is rejected from the further processing.

A method for determining a wall thickness, T will now be described. The inner wall location and the outer wall locations previously determined (see FIG. 21A and FIG. 21B) may be used to find the wall thickness by forming a difference between the outer and inner wall locations:

T=(Outerwall−Innerwall)·RF_resolution

RF_resolution is the length of a single RF sample, typically but not exclusively 0.08 millimeters. Since a plurality of scancones are developed during an ultrasound examination, and each scancone has a pair of orthogonal planes having corresponding thickness estimations, a median value may be calculated and accordingly constitutes a best estimate of the wall thickness.

FIG. 22 is an exemplary graph of a cost function generated along a selected scanline, which was employed in the methods described in FIG. 21A and FIG. 21B. The cost function is thus minimal at a final outer wall layer location exhibiting minimum thickness values. The cost function may therefore be used to identify the minimum thickness value since it is proximate to the minimum cost value.

FIG. 23 is an exemplary scanplane 42 of an internal anatomical region having a sector of scanlines 48 superimposed on the scanplane 42. The scanlines 48 cross an inner layer border initially determined by the two-dimensional B-mode segmentation algorithms discussed above, in connection with FIGS. 14 and 15. The initially determined inner layer border provides a first wall location from which, at the scanline level, a one-dimensional A-mode algorithm may be applied to rectified RF envelopes to determine the nearest outer layer candidates and the nearest inner layer candidates. Either at the scanplane or scanline level, the nearest outer layer candidate points amount to a second wall location. Similarly, the nearest inner layer candidate points amount to a third wall location.

FIG. 24 is an expanded portion of the scanplane 42 of FIG. 23 that shows the initial front wall location in greater detail. The expanded portion of the scanplane 42 shows the outer and inner layer borders of the initial front wall location as determined by the one-dimensional A-mode algorithms with the two-dimensional B-mode inner layer border. Compared with FIG. 19, six of the outer layer candidates were eliminated leaving the nearest outer layer boundary line as shown. The nearest outer layer boundary amounts to the second position loci. Also shown is a nearest inner layer boundary displaced anteriorly to the initial front wall boundary layer as determined from two-dimensional segmentation algorithms.

FIG. 25 expands sub-algorithm 172 of FIGS. 14 and 15. The sub-algorithm 172 is comprised of three blocks. In block 172A, the patient is palpated to determine the location of the synthesis pubis or as commonly known the pubic bone. Above the synthesis pubis location, a sonic gel pad or a sonic gel is applied and the scanner is either placed in the gel that is applied to the patient or on the sonic gel pad. The sonic gel and the sonic gel pad serve to minimize attenuation of the ultrasound that transverses between the transceiver dome 20 of the transceiver 10 and the patient. The next block is 172C and the scan button is pressed on the transceiver 10 so that a rotational array of 2-D scanplanes is acquired. The method then proceeds to block 176 from FIG. 14.

FIG. 26 expands sub-algorithm 180 of FIG. 14. The sub-algorithm 180 is comprised of eight process or decision routines. The first process is block 180A and is called Find Initial Wall. From block 180A is the next block 180B that is Find Centroid. Thereafter, block 180C is Fixed Initial Walls. After Fix Initial Walls is a decision block in which the question is asked, “Is it uterus?” The decision block 180D. If it is a uterus, “yes”, the next process is Clear Walls block 180E. Thereafter, the volume is displayed at in process 180H and the process continues on to process 180J. Referring back to decision diamond 180D, if the organ is not a uterus, “no” then we proceed to decision 180F in which the question is asked, “Is volume less than 40 ml.?” If the answer is “no” to the decision diamond 180F, then the volume is displayed at terminator 180H and the algorithm then proceeds to sub-algorithm 180J. If at decision diamond 180F the answer is “yes” to the query, “Is volume less than 40 ml.?” Another decision diamond is presented 180G. At decision diamond 180G, the query is asked, “Is it a bladder region?” If the answer is “no” then the sub-algorithm 180 proceeds to the Clear Walls of block 180E and thence to terminator 180H Volume Displayed. If at the decision diamond 180G, the answer is “yes” to the query, “Is it a bladder region?” then the volume is displayed at terminator 180H and the process then continues on to algorithm 180J. In sub-algorithm 180, an interface line is overweighed on the B-mode scanplane image to approximate an initial location for an organ wall, for example, a uterus or a bladder. This initial interface line is used as a seed or initial reference point in which to further use as a basis to adjust the determination for the inner and outer wall layers of the organ wall. Furthermore, in this algorithm, the detected region in the scanplane is determined to be or not to be a bladder or a uterus. This occurs specifically when the gender button of the transceiver 10 indicates that the scan is for a female. If the regions indeed found to be a uterus, it is cleared and a zero volume is displayed. For a non-uterus region, such as a bladder, if the volume is very small, then checks are made on the size of a signal characteristic inside the detected region to ensure that it is a bladder and not another tissue. If a region is indeed a bladder region it is computed and displayed on the output.

FIG. 27 expands sub-algorithm 180A of FIG. 26. The sub-algorithm 180A is comprised of 11 processes loops, decisions, and terminators. Sub-algorithm 180A begins with process 180A2 in which the Local Average is calculated for the 15 to 16 samples that functions as a low pass filter (LPF) to reduce noise in the signal. Other embodiments allow for calculating averages from less than 15 and more than 16 samples. Next is block 180A4 in which the gradient is calculated using a central difference formulation and has taken over seven sample sets. The process at block 180A4 then proceeds to a beginning loop limit 180A6. In block 180A6, each sample is examined in a detection region. Thereafter, at decision diamond 180A8, the query is, “Is gradient minimum?” If the answer is “no” then another query is presented at decision diamond 180A18, the query being, “Looking for BW and gradient maximum?” BW refers to for back wall. If the answer to the query in block 180A18 is “no” then the end of the loop limit is proceeded to at block 180A30. Thereafter, from the end of the loop limit at 180A30, the terminator end find initial walls is reached at block 180A40. Returning now to the decision diamond 180A8, if the answer to the query, “Is gradient minimum?” “yes” then another query is presented in decision diamond 180A10. The query in 180A10 is “Is candidate FW/BW best?” FW is refers to front wall and BW refers to back wall. If the answer to the query in block 180A10 is “no”, then the process 180A62 is used in which the front wall is saved and another back wall is looked for. If the query to in 180A10 is “yes” then the process is Save Candidate occurs at block 180A14. Thereafter, the process returns to beginning loop 180A6 to resume. Returning to the decision diamond 180A10, should the answer be “yes” to the query, “Is candidate FW/BW best, then the process proceeds to block 180A12 in which the candidate is assigned as a pair for back wall/front wall.” Thereafter from block 180A12 is returned to the beginning loop 180A6 and then the process will then terminate at end of each sample at end loop 180A30 and thence to terminator 180A40 for end find initial walls sub-algorithm. Sub-algorithm 180A attempts to find the best front wall and back wall pair for the inner and outer wall layer plotting points. The best front wall and back wall pair in each scanline is defined as the front wall and back wall pair for which the difference in the back wall gradient and front wall gradient sometimes referred to as the tissue delta, is the maximum and the smallest local average between the front wall and back wall pair is the minimum for the pixel values.

FIG. 28 is an expansion of the sub-algorithm 180C of FIG. 27. Sub-algorithm 180C is comprised of several processes decision diamonds and loops. Sub-algorithm 180C operates on a scanplane by scanplane basis where the first scanplane to be processed is one that is closest to the central aid of the initial walls and then the remaining scanplanes are processed moving in either direction of that initial scanplane. Sub-algorithm 180C begins at block 180C2 referred to as Start Fix Initial Walls. The first process is at block 180C4 in which the center line is corrected if necessary. The center line is defined as the line on that scanplane with the maximum gradient difference between the front wall and the back wall. The correction of the front wall and the back wall location at any line is carried out by a match filtering like step where the best location within a search limit is defined as the one for which the difference between points immediately outside the bladder and points immediately inside the bladder is maximum. Of course, this applies to any organ other than the bladder, as the bladder is used here as an example of a particular embodiment. Thereafter, at block 180C6, the front wall and back wall means are calculated for five central lines. The pixel main intensity is computed and if this intensity is less than expected from the noise at that depth, the lines are cleared and the algorithm proceeds to the next plane as shown in decision diamond 180C8 to the query, “Is BW level less than noise?” where BW means the back wall (or posterior wall) of the bladder. If the answer is “yes” to this query, at block 180C10, the process Clear Wall Data is initiated and from that proceeds to terminator 180C50 End Fix Initial Walls. Returning to the decision diamond 180C8, if the answer is “no” to the query, “Is BW level less than noise?” then the sub-algorithm 180C proceeds to the process at block 180C12 described as Fix 3 Central Lines. From this point through the end of sub-algorithm 180C, the purpose is first correct the lines to the left of the central lines, called the left half plane (LHP) until either the edge of the bladder or the edge of the ultrasound cone is found. After the algorithm corrects the LHP, it proceeds to correct the lines to the right of the central lines, called the right half plane. Because the same steps are used for all lines, regardless of their position to the left of center or to the right of center, the process blocks 180C16 through 180C42 are used for both the LHP and once for the right half plane. The “line index” of process 180C14 indicates an identifier for the current line that is processed. The line index is set to 2 indices less than the center line to start processing the LHP. The looping procedure started in block 180C16 continues looping while the line index is a valid index (i.e. it corresponds to a scanline). Sub-loop 180C18 is started with the intent of adjusting the initial wall locations, sub-process 180C20, to their correct location if any correction is necessary. This loop, terminated at process 180C24, completes two iterations. The first iteration uses sub-process 180C20 to correct the front wall of the bladder on the current line and the second iteration to correct the back wall of the bladder, although the ordering of which wall is corrected first can be interchanged. Once the wall locations have been corrected of the current line have been corrected, sub-algorithm 180C proceeds to sub-process 180C28, “Check Wall Growth”. This sub-process ensures that the length of the scanline that intersects the bladder in the current line does not grow significantly with respect to the previous line that has already been corrected. In the preferred embodiment, the length of the scanline intersecting the bladder is constrained to be less than 1.125 times longer than in the previous line. If the loop bounded by sub-processes 180C16 and 180C42 is being applied to the LHP, then the previous line is one index number greater than the current line index. Otherwise the previous line index is one index number less than the current index. After completing sub-process 180C28, sub-process 180C30 “Check Wall Consistency” verifies that the portion of the current scanline that intersects the bladder overlaps the portion of the previous scanline that intersects the bladder. After completing sub-process 180C30, decision 180C32 queries “If working LHP?” (i.e. the loop bounded by terminators 180C16 and 180C42 is being applied to the lines left of center). If the answer to the query is yes, then the sub-process 180C34 “Decrement line index” decreases the line index by one index number. Decision 180C36 queries “If line index is invalid”. The loop bounded by terminators 180C16 and 180C42 is applied to the next, and now current, scanline. If the decremented line index corresponds to an invalid value, the edge of the LHP has been reached. Sub-process 180C38 is called to reset the line index to the first line to the right of center that has not been adjusted. The loop bounded by terminators 180C16 and 180C42 will now be applied to the right half plane (RHP). Returning to decision 180C32, if the answer to the query is “No”, sub-process 180C40 “Increment line index” results with the line index being increased by one index number. Loop terminator 180C42 cause the loop to return to 180C16 as long as the line index corresponds to an actual scanline. As soon as that condition is violated, the loop terminator will cause sub-algorithm 180C to proceed to the terminator 180C50, “End Fix Initial Walls”.

FIG. 29 is an expansion of the sub-algorithm 180J of FIG. 26. The procedures within sub-algorithm 180J provide a decision tree used for ascertaining whether a uterus has been detected. The definitions of the abbreviations in the flow chart blocks are Max E, Max V1, Max V2, ValMean, and MaxVM. Max means maximum, E means enhancement, V1 means volume 1, V2 means volume 2, ValMean refers to a measurement of the minimum local average pixel intensity of the region inside the region identified as urine inside the bladder, Max VM is a pre-defined threshold against which VALMEAN is tested. If VALMEAN is greater than MAXVM, the region identified as urine inside the bladder isn't really urine and the boundaries are actually an outline of the uterus. Depending on the hardware platform used for the various embodiments of the transceiver 10, the decision tree for the sub-algorithm 180J of FIG. 26. The sub-algorithm 180J begins from sub-algorithm 180H in which a decision diamond Enhancement<MaxE (maximum enhancement) at decision diamond 180J2 is reached. If the answer is “yes” for enhancement, then another decision diamond 180J4 is reached and the query is a Volume<Max V1 (maximum Volume 1) is made. If the answer is “yes” to this query, then the determination at terminator 180J6 is reached and the organ that is being examined is a uterus. Thereafter, the algorithm continues to block 184 of FIG. 14. Returning to the decision diamond 180J4, if the answer is “no” to the query Volume<Max V1, then another decision diamond 180J8 is reached having the query “Is the Volume<Max V2?” (Maximum Volume 2). If the answer is “yes”, then the next decision diamond is 180J10 is reached with the query, “Is the ValMean>MaxVM?” If the answer is “yes”, then terminus 180J6 is reached and the organ being viewed is the uterus. If the answer is “no”, then terminus 180J20 is reached and the organ being viewed is a bladder, the algorithm then completes block 184 of FIG. 14. Returning back to decision diamond 180J8, if the answer is “no” to the query, “Is the volume<than MaxV2”, then the answer is that a bladder is being viewed as indicated by the terminal 180J20.” From terminus 180J20 the algorithm continues to block 184 of FIG. 14.

FIG. 30 is an expansion of an alternate embodiment of the sub-algorithm 184 of FIG. 14. The processes within sub-algorithm 184 are procedures taken between blocks 180 and 188 of FIG. 31. The sub-algorithm 184 is comprised of block 184A2 in which 1-D scanline signals are examined for scanlines crossing the organ wall. Thereafter at block 184A4, the echo signals are rectified using a Hilbert Transform to obtain an A-mode radio frequency (RF) envelope along scanlines crossing the organ wall. Sub-algorithm 184 continues with block 184A6 where the scanline RF envelope is examined for candidate points of inner and outer wall layers of the organ wall. Thereafter at block 184A10 the candidate points are plotted for the inner and outer wall layers of the organ wall on scanlines within the 2-D scanplanes. Finally, the sub-algorithm 184 is completed with the process described at block 184A12 in which the best candidate points are determined for the inner and outer wall layers of the organ wall being examined on scanlines using a least cost analysis algorithm previously described above.

FIG. 31 is an expansion of the sub-algorithm 188 of FIG. 14. Sub-algorithm block 188 is between sub-algorithms 184 and 192 of FIG. 14. There are two sub processes in 188 depending upon how the organ wall thickness is calculated depending upon either a single value or a group of values. For a single value at block 188A2, the organ wall thickness is calculated as a difference between one pair of best inner and outer layer wall candidates from one scanline. Alternatively, at block 188A4, the organ wall thickness is calculated as a mean of the differences between a plurality of best inner and outer wall layer candidates pairs of more than one scanline crossing the organ. Both blocks 188A2 and 188A4 are then continued to sub-algorithm 192.

FIG. 32 is an expansion of the sub-algorithm 192 of FIG. 31. Sub-algorithm 192 is between sub-algorithm 180A and thickness measurement 188. Sub-algorithm 192 starts with block 192A morphological cleanup. The processes of sub-algorithm 192 identifies potential front wall and back wall pairs on A-mode scanlines that potentially look like an organ of interest, for example, a bladder in which a dark region which is surrounded by bright echos on the front and of the back of the organ being viewed. The sub-algorithm 192 uses some shape and anatomical knowledge to clean up the potential front walls and back walls in the morphological cleanup block 192A. The morphological cleanup is needed because there may be missing wall pairs that appear spurious and further more are further obscured by speckle and other noise associated with ultrasound-based images. Such a speckle and other ultrasound-based noise may give a front and back walls that are unnecessarily jagged. The morphological cleanup at block 192A serves for correcting errors due to this jaggedness and for regularizing or smoothing these wall locations. The morphological cleanup block 192A uses mathematical morphology and a sequence of morphological operations that are applied to the initial wall data. The mathematical operations will be described in figures below. After execution of the morphological cleanup process at block 192A, there may be more than one potential region that represents an organ of interest say the bladder. If there is more than one region, then the largest three-dimensional region is assumed to be the bladder and is selected for further processing. This selection of the largest region occurs at the next block 192B. After the largest region selection is determined, another smoothing and cleanup process is applied at block 192C mainly a process referred to as snake smoothing. A variant of the snake-smoothing algorithm was developed and is described in the figures below. The boundary output from this snake smoothing algorithm step 192C is used to calculate the surface area of the bladder using an algorithm described below. The initial points that are used in sub-algorithm 192 are those that were already obtained to have high confidence. Those that were not high confidence wall points are filtered and removed. The high confidence front wall locations are then used to initialize the RF base thickness measurement as described above and as further elucidated below. Parallel with the snake smoothing algorithm 192C, a block 192D is implemented in which high confidence front walls are selected or chosen. After snake smoothing has been implemented at block 192E surface area measurement is then conducted.

FIG. 33 is an expansion of the sub-algorithm 192A of FIG. 32. Several steps are applied to initial wall data. A series of morphological openings and closings are used with increasingly large kernel sizes and are applied to the pre-scan converted data. This kind of filter is known as “alternating sequential filter” and further described in P. Soille and J. F. Rivest, Principles and Applications of Morphological Image Analysis. In the expansion of sub-algorithm 192A, gaps are filled between planes and the image sequence. As an example, the sub-algorithm 192A is represented by a series of close and open processes that are shown in eleven process boxes and conclude with an erode box. The first close process box is 192A1 which then proceeds to a first open process box 192A2 and further proceeds to the following series described below. The series of morphological openings and closings are used with increasingly large kernel sizes and are applied to the pre-scan converted data. The first operation is a closing with a structuring element 3 planes deep designated in block 192A1 as 1×1×3. This step fills in the gaps between planes that extend to less than 3 planes. Next, in open block 192A2, a structuring element 3 planes deep is opened which removes outlier regions between the planes that extend for less than 3 planes. Thereafter, at block 192A3, the data is closed in a 1×1×5 sequence and then reopened at block 192A4 in a 1×1×5 sequence. That is the structuring elements of 5 planes deep in blocks 192A3 and 192A4. The open and close algorithm continues with open block 192A5 and close block 192A6 in which this series of morphological operations aim to fill gaps and remove outliers within a plane. In open block 192A5, a small opening using a structure element 3 scanlines wide is implemented and this serves to remove outliers that are less than 3 scanlines wide. This step is then followed by block 192A6 in which a closing process is implemented that closes all gaps in the wall locations less than 3 scanlines wide. Thereafter, another open and close pair of processes are applied at open block 192A7 and close block 192A8. The open block 192A7 is of a 1×5×1 configuration and the close processing block 192A8 is of a 1×5×1 operation. Thereafter, an open and close processing is done in a 1×7×1 configuration at block 192A9 and block 192a10, respectively. In these two blocks, outliers are removed and gaps are filled for 5 and 7 scanlines, respectively. Thereafter, at open processing block 192A11, a 15×11×1 configuration is implemented in which 15 samples long and 11 scanlines wide are processed to help select for the proper points. In open block 192A11, the main purpose is to remove erroneous front wall locations that are affected by the dome reverberation artifact dissociated with ultrasound echo reverberations of the dome 20 if the transducer 10. The final step of sub-algorithm 192A is an erode processing block 192A12 in which the morphological processing erodes the front and back walls by 5 samples. That is, this is a 5×1×1 configuration in which the step shrinks the front walls and the back walls inside to allow the snake to expand and search for the best location.

FIG. 34 expands sub-algorithm 192C of FIG. 32. Sub-algorithm 192C is for snake smoothing and is comprised of several processing and terminator steps. Snake processing uses an active contour known as a snake and is basically a way to link edges or other image features by minimizing a cost function for a contour passing through the image features. The cost function typically includes a cost that favors contours that are close to the desired image features on the image and a cost that favors smooth and short contours.

The minimum cost contour is found by using an iterative method starting with an initial contour that is fairly close to the desired contour. This initial contour is minimized iteratively and the motion of the contour between iterations resembles the motion of a snake; therefore the name of the algorithm. The snake moves under two forces—(1) an image-based force that tries to move the contour closer to image edges, and (2) a regularizing force that tries to make the contour smooth and short. At the end of the iterations, a contour is developed which balances the two forces using the following sub-algorithms of snake smoothing algorithm 192C of FIGS. 32 and 34.

A combination of two images is used to define image-based forces. The first image is a gray scale image that is inputted at starting terminus 192C4. Thereafter, a heat and shock filter at block 192C6 is applied which respectfully serve to optimize a detection of the gray scale image. The two images are incorporated into the snake metric using the following logic. Looking along a direction normal to the snake curve, the optimal snake location has the maximum difference between the gray scale intensities outside the curve and the gray scale intensities inside the curve and it lies on a location that is identified as an edge point. This occurs at the edge detection process block 192C8. After heat and shock filtration at block 192C6 and after edge detection at 192C8, a 2-D snake algorithm is applied as described further in block 192C10 of FIG. 34. At 192C10, an initial bladder outline or other organ of interest outlined is provided to processing block 192C10. The initial bladder outline is inputted from input terminal 192C2. After application of the 2-D snake process 190C10 to the input 2-D scanplane image of 192C4, an overlay with initial bladder outline of 192C2, a final bladder outline is generated at terminus 192C20. Discussing below an amplification of the 2-D snake algorithm 192C10 is further described.

FIG. 35 expands sub-algorithm 192C10 of FIG. 34. The expansion of this algorithm serves to make the snake an iterative sequence of the following two steps—(a) moving the contour in a direction normal to the contour where each normal direction that is searched becomes the best image metric, and (b) smoothing the deformed contour using regularization constraints. In the application of the sub-algorithm, each point along the curve is examined and image pixels are sampled normal to each point and the image metric is calculated at each normal location within a pre-specified search range. Thus, beginning at the loop at 202, each point of the curve is readied for processing. Thereafter at processing block 204 a normal to the point on the curve is found. Thereafter at block 206, a normal to the image metric is computed provided that filtered images from block 216 and edge image 218 are available. The image metric at each point uses the gray scale pixel intensities inside and outputs the curve and also uses the edge image obtained respectfully from the filtered image block 216 and edge image block 218. The contour point is moved to a location where the image metric is optimal, i.e., the gray scale intensity difference is maximal and the location corresponds to an edge location. This is denoted in block 208 selected best location. Thereafter, the processing loop is ended at block 210 and the processing points on the curve is completed. Next is a smoothing of a contour that is carried out at block 212, smooth curve. Of the contour, it is carried out by multiplying the vectors representing the X and Y coordinance of the contour with a smoothing matrix. Following the smooth curve 212 block is a decision diamond for the termination of the Max iterations has been reached and if it has, then the 2-D snake algorithm 192C10 is completed at terminus 220. If it has not, the procedure returns to the opening loop 202 of the sub-algorithm. Referring now to the filtered image block 216 of the edge image block 218, the snake algorithm are applied to obtain the best computed image metric along the normal block 206 based upon examining every detected front wall layer location within a small search region on the same scanline around the detected front wall layer location. If no edges are found within the search area, the wall location is considered of low confidence and is removed from the output wall locations. However, if an edge point exists within that search region, and the intensity difference between the pixels outside and inside the organ wall, for example, a bladder wall on an enhanced image is maximal, the location is considered a high confidence location. The output wall location for such a point is moved to this high confidence location.

FIG. 36 expands sub-algorithm 192E of FIG. 32. Sub-algorithm 192E concerns the procedures for obtaining a surface area measurement and comprises a series of processing steps. Starting with block 192E2, the segmented front and back walls are supplied to a fill bladder region procedure in block 192E4. The fill bladder region procedure creates a pre-scan converted, for example, in polar coordinate form, volume where all the pixels inside the bladder are filled in with a non-zero pixel value such as 255. Then all the pixels outside are set to zero. The next procedure is in block 192E6, a 3-D scan convert process. The 3-D scan convert process is a conversion procedure applied to convert the polar coordinate pre-scan image to a Cartesian coordinate system. The size of the Cartesian volume created is 150×150×150. This Cartesian volume data is then smoothed as indicated in block 192E8 3-D image smoothing. The smoothing step uses a Gaussian smoothing window of approximately 11×11×11 pixels. The kind of filtering used in the Gaussian smoothing is preferable to generate a smooth output organ surface as would be for a bladder surface. In the next block 192E10, a general iso-surface procedure is implemented. The general iso-surface procedure uses the Marching Cubes algorithm described in Lorensen and Cline (W. E. Lorensen and H. E. Cline, “Marching Cubes: A High Resolution 3D Surface Construction Algorithm,” Computer Graphics, vol. 21, pp. 163-169, July 1987.) Marching Cubes algorithm is applied to create iso-surface of the organ region such as a bladder. An iso-value of 127.5 is used to decide where to place the iso-surface on the smooth image. Everything greater than this iso-value of 127.5 is considered inside the bladder or the organ of interest and less than this value is considered outside the bladder or organ of interest. In the next step process 192E12, the organ surface is then decimated and smoothed to reduce the number of vertices. The surface is then triangulated at process step 192E14 in order to represent the entire surface using a mesh of triangles. This triangulated surface is then outputted as a VRML for potential display and is also used for the calculation and surface area and other properties. The triangulated surface is used for surface area calculation. As shown in the FIG. 36, the triangulated surface is also output as a VRML file in terminus 192E16. The surface properties, surface area, etc. are calculated as indicated in block 192E18. Thereafter, at terminus 192E20, the surface area is outputted for report.

FIGS. 37A-D are B-mode scans overlaid with interface tracings obtained by the algorithms previously described. FIGS. 37A and B are sagittal plane (plane 1) images and FIGS. 37C and D are transverse images. A line along the back wall in FIG. 37A is seen and a more jagged line in FIG. 37B is shown as a consequence of noisy signals. FIGS. 37C and 37D show the cleanup of the interface tracings along the organ wall boundaries, in this case a bladder after being subjected to the morphological cleanup, sub-algorithm 192A of FIG. 32. Note the loss of the jagged interface tracings of 37B substantially smooth over and as an interface tracing 37D.

FIGS. 38A-D are B-mode scans overlaid with interface tracings before and after application of the morphological cleanup algorithms. As with FIGS. 37A-D, FIGS. 38A and B are sagittal images and FIGS. 38C and D are transverse images. Again, note the difference between FIGS. 38B whether a substantial jagging along the back wall that clearly goes into the tissue and whereas morphological cleanup there is a substantially closer interface tracing along the boundary of the organ wall, in this case, a bladder along the back wall of the bladder.

FIGS. 39A-D are B-mode scans overlaid with interface tracings. This is yet another iteration of the morphological cleanup process in which a truer fidelity is achieved demarcating in this 2-D scan images a more precise interface tracing demarcating the bladder from surrounding tissue after application of the snake algorithms.

FIGS. 40A-B are normal and magnified B-mode scans overlaid with interface tracings. FIG. 40A is a normal view and has a white square looking at the bladder wall area. FIG. 40B is an expansion of the white square perimeter of FIG. 40A in which the inner and outer wall layers are shown delineated as separate tracings. There is a high degree of resolution by using the algorithms of the preceding as discussed previously.

FIGS. 41A-B are normal and magnified B-mode scans overlaid with interface tracings. Similar to the tracings of FIGS. 40A and B, a normal view of FIG. 41A is shown with an enclosed square which is magnified in the FIG. 41B to show comparable high resolution interface tracings of the inner and outer wall layers of the front wall organ wall, in this case, a bladder. The front wall muscles as detected for the bladder wall in FIG. 40B and FIG. 41B are used for the thickness calculation measurements of FIG. 32.

FIG. 42 is an alternative-algorithm of FIG. 15. Raw data is first brought under processing block 172 and thereafter the raw data is split between segmentation of the organ and calculate wall area block 192 and finding the extent (search region) of proximate organ wall to the transceiver block 250. After block 250, the inner wall layer of proximate organ wall to transceiver is achieved at block 252. Thereafter, at block 254, find outer wall layer of the proximate organ wall to transceiver is implemented. Thereafter, at block 256, the thickness of the proximate wall as a difference between the inner and outer wall layers is then calculated. Thereafter, the two parallel fracture combined merge at block 300 in which the organ wall volume is calculated and thereafter ends with block 400 in which the organ wall mass is calculated. All the processing here—250,252,254,256 is carried out on 2D or 1D data.

FIGS. 43A-B are B-mode scans overlaid with interface tracings. A scanplane 500 is shown having a bladder 500C which is delineated along its tissue cavity boundary by a front bladder wall 500A and a back bladder wall 500B. FIG. 43B is another scanplane from the same patient and shows the initial wall locations of a scanplane 502 about the bladder 502C in which the front wall 502A and back wall 502B is delineated by interface tracings.

FIGS. 44A-B are B-mode scans overlaid with interface tracings. FIG. 53A is a scanplane 506 and 53B shows a scanplane 508 from the same patient. In contrast to the scanplane in FIGS. 43A and B, the boundaries are more difficult to set with the tracings and show that parts of the bladder as delineated as 506C and 508A, respectively are comparably delineated with 506A as the front wall and 506B as the back wall in scanplane 506. Similarly, the delineation of partial where only part of a front wall 508A is shown as a interface tracing and part of the rear wall 508B is shown as interface tracing. In this case here in FIGS. 43A and 43B, would receive the benefit of filling in the likely candidate points in the gaps set are between the front and rear wall interface tracings.

FIGS. 45A-B are B-mode scans overlaid with interface tracings. The interface tracings for scanplanes 510 and 512, respectively of FIGS. 45A and B show a partially delineated bladder that goes off scale. The bladder is respectfully represented as 510C and 512C and the figures and the respective front walls are 510A and 512A and the rear walls are 512B and 510B. Of interest to note is that using the method the algorithms of the system is that the outer wall layer and inner wall layer is more clearly delineated. The outer wall layer in scanplane 510 is shown as 510D and the inner wall layer is shown more clearly as 510A for the front wall. The rear wall does not shown this delineation with tracings at this point.

FIGS. 46A-B are B-mode scans overlaid with interface tracings. The interface tracings as shown in the previous FIGS. 46A and B show the front wall tracings for the inner and outer wall layers. In scanplane 514 of FIG. 46A, the outer wall layer of 514D is shown and inner wall layer 514A is shown of a partially revealed bladder. Also shown in scanplane 514, is the partial back wall delineation along tracing 514B. In FIG. 46B, scanplane 516 shows a slight proportion of the inner wall layer of 516A and the outer layer of 516B and only a very small portion of the back wall 516B.

FIGS. 47A-B are B-mode scans overlaid with interface tracings. FIG. 47A concerns scanplane 518 and FIG. 47B concerns scanplane 520. In scanplane 518, the outer layer wall of 518D may be seen traced with the inner layer wall 518A. The back wall 518B is shown partially traced. In FIG. 47B scanplane 520 is sequential with scanplane 518 of FIG. 47A and another view of the delineated bladder may be seen. Due to the differences in the scanplanes, the relatively full bladder may be seen where the proximate or forward bladder wall is seen delineated with the inner wall 520 and the outer layer wall 520D. Also, visible is the delineation for the back wall 520B that goes off image.

FIGS. 48A-B are B-mode scans overlaid with interface tracings using the preceding algorithms. FIGS. 48A and 48B present to a sequential scanplane from a different patient with different views of the bladder available. In scanplane 522, the inner layer 522A of the proximate or forward bladder wall is shown delineated and the outer layer 522B is shown delineated with the interface tracings. The back wall 522B is shown slightly delineated and off image. Similarly, FIG. 48B shows scanplane 524A with only a portion of the bladder visible, but nevertheless the inner layer 524A is shown with the interface tracing along with the outer layer 524D with an interface tracing for the proximate forward bladder wall. Only a portion of the back wall of 524B is visible.

FIG. 49 is a method algorithm for the Internet System used to measure organ wall mass. In FIG. 49, the exam valuation is for a BVM 6500 transceiver end block 600. Block 600 is composed of a user block 600A, a sonographer block 600B, and ScanPoint database block 600C, and a ScanPoint application block 600D. The Internet system 600 uses a coordinated interplay between the user block 600A dismounted for 600B via database ScanPoint software 600C and the ScanPoint application software of 600D. The user begins the exam evaluation 600 by scanning the patient at procedural block 600A2. Thereafter, at procedural block 600A4, the exam is off-loaded to the ScanPoint server. The ScanPoint server block 604 receives the analysis and stores the results from the exam uploaded from block 600A4. Thereafter, the results are saved in the ScanPoint database 600C at procedural block 606. A sonographer experienced to review the images and results from the ScanPoint database 600C at procedural block 606 reviews the exam at block 608. Thereafter, a decision diamond 610 occurs in the sonographer column 600B where the query is as presented, “Are the automated results good?” If the answer is “no”, the another decision diamond is presented at 614 with the query, “Can the exam be corrected?” If the answer is “yes” to the query in decision diamond 614, at block 615, the results are edited and submitted for re-analysis. Returning back to decision diamond 610, if the answer is “yes” to the query, “Are the automated results good?” the procedure returns to the ScanPoint database 600C column where at block 612 the exam is marked available to user. Upon successful assessment by a sonographer at decision diamond 610, and after being marked available for user at procedural block 612, the exam results are made available to the user at block 624 wherein it then becomes accepted by the user for evaluation at block 628 within the user column 600A. In block 628, the user accepts or rejects the results after the sonographer has approved it. The accepted to rejected results from procedural block 628 is then sent to the ScanPoint database and stored at procedural block 640. In the ScanPoint 600C. Returning to the sonographer, column 600B at procedural 615, there are two options that occur at ScanPoint database 600C and ScanPoint application 600D. In the ScanPoint database column 600C, a procedural block 616 for clone exam is available. In the cloned exam procedure, the exam may be repeated as desired by the sonographer. Alternatively, in the ScanPoint application software 600D at procedural block 618, the results of the scan may be analyzed and stored. Thereafter, returning to the ScanPoint column 600C, the results that are saved are marked clone and the exam results are made available to the user. Thereafter, at block 624, the exam results are made available to the user and from block 624 and the user column 600A the user then reviews the exam results at block 628 and decides to accept or reject the results. Thereafter, the user returns to the ScanPoint database column 600C and the results are saved and a charge for the exam is made if necessary procedural block 630. From preceding detailed description of the major operation processes of the exam evaluation 600, it can be seen that the bladder mass exam is deployed via the Internet system as a reviewed exam. An experienced sonographer reviews the exam and the resulting data is re-analyzed as needed. As shown in the user column 600A, the user is free to scan a patient prior to after preparing an exam in ScanPoint. In starting block 600A2, the user performs the following steps substantially similar to that described in sub-algorithm 172 of FIG. 15. First, the patient is palpated to determine the location of the symphysis pubis or the pubic bone approximately two centimeters or one inch above the patient's symphysis pubis along the patient's midline the transceiver 10 is placed. Prior to that, either a sonic gel pad is placed at this location or a ultrasound conveying gel is applied to the patient's skin. Thereafter, the transceiver 10 currently a BVM 6500 scanner is placed in the center of the gel pad or near the center of the applied gel. Then, the scan button is released to acquire the rotational array of 2-D scanplanes referred to as V-Mode™ scan. Once the V-Mode™ scan trademark is completed, the results are conveyed as indicated in the flowchart of FIG. 49. The particular embodiment to the transceiver 10 specifically the BVM 6500 scanner can notify the user, through display presented arrows, whether or not the aim of transceiver 10 needs to be adjusted to acquire the organ of interest, in this case a bladder, so as to acquire the bladder in a more reasonably centered location. Attempts to prove the aim at this point are recommended, but optional. That the organ of interest in this case, a bladder is properly centered is verified by getting consistent readings through multiple repositions. It is suggested that at least three volume readings be acquired that are consistent. As previously indicated in the Internet system method of FIG. 49, the exams uploaded to the ScanPoint software and is available soon thereafter for review by a sonographer. The sonographer reviews the raw data uploaded to the ScanPoint database and analyzes the organ volume surface area and wall mass. The sonographer can assess the results as is, reject the exam outright, or edit the exam. If the sonographer accepts or rejects the exam, the result is immediately available to the user. If the sonographer chooses to edit the exam, a new window opens on the computer display. In this window, sonographer will trace inner and outer bladder wall layers on both the sagittal plane and on the transverse plane by selecting a series of points. These measurements will be uploaded to the ScanPoint software database and ScanPoint application. The ScanPoint Internet system will clone the exam results to form a new record and raw data will be re-analyzed with the sonographer's measurement locations. The sonographer's measurement will be added to the zoom thickness measurements after this repeated analysis. After the re-analysis is complete, the results corrected by the sonographer will be presented to the user along with the original thickness measurement result. At this point, the user is free to view the results. The user may accept or reject the exam in the same manner as other exams available and the ScanPoint suite.

FIG. 50 is a screen shot of four image panels A-D. The screen shots are what is available to be seen by the user or sonographer after his points along the execution of the Internet algorithm as described in FIG. 49.

FIG. 51 is a screen shot of two image panels A and B. The screenshot as shown shows two other image panels with two inner face tracings drawn in image B. The two images here are editable as needed.

FIG. 52 is a screen shot of six image panels A-F. The six screen shots are acquired and show different degrees of image processing and overlaying of interface tracings for the outer at inner wall layers of the proximate or forward organ wall.

FIG. 53 is a screen shot of Exam Quality Report. The Exam Quality Report has different test options including bladder mass, bladder volume, amniotic fluid volume, etc., as well as different levels of descriptors that categorize whether the particular exam selected is % incomplete or % inconclusive, the number of exams, the percent good they were, the amount of % that are quality assurance rejected or % that are quality assurance edited, or the number in which the user has rejected.

FIG. 54 is a screen shot of two image panels A and B indicating initial segmentation of a bladder. The bladder that has been segmented is an early stage of organ wall tissue interface resolution as indicated by the relative jagged interface tracings.

FIG. 55 is a scanplane image overlaid with inner and outer wall tracings using algorithms of the Internet System. An outer wall layer 920 is shown in relation to an inner wall layer tracing 922. While preferred and alternate embodiments of the invention have been illustrated and described, as noted above, many changes can be made without departing from the spirit and scope of the invention.

Certain embodiments of the present invention relate to and can be practiced in conjunction with the invention and embodiments described in our co-pending application filed via Express Mail Label No. EV510340886US on Feb. 17, 2005, which is hereby incorporated by reference.

Methods and systems to acquire an ultrasound estimated organ wall mass and/or weight such as a bladder using three dimensional ultrasound echo information are described. The three-dimensional (3D) based ultrasound information is generated from a microprocessor-based system utilizing an ultrasound transceiver that properly targets the organ or other region of interest (ROI) and utilizes algorithms to delineate the inner (sub-mucosal) and outer (sub-serosal) wall boundaries of the organ wall as part of a process to determine the organ wall weight or mass. When the organ is a bladder, bladder wall algorithms operate without making geometric assumptions of the bladder so that the shape, area, and thickness between the sub-mucosal and subserosal layers of the bladder wall are more accurately determined to provide in a turn a more accurate determination of the bladder wall volume. Knowing the accurate bladder volume allows a more accurate determination of bladder wall weight or mass as a product of bladder wall volume and bladder wall density or specific gravity.

The embodiments include a system and methods for an automatic and convenient procedures to obtain an estimated bladder weight (UEBW) and/or a mass of the bladder wall based on analysis of three-dimensional images and analysis of one and two-dimensional information that comprises the 3D image. In one particular embodiment, a subject or patient is scanned using an ultrasound transceiver. The ultrasound transceiver, similar to the BladderScan® BVM6500 marketed by Diagnostic Ultrasound Incorporated of Redmond, Wash. provides an ultrasound sound image in the form of a three-dimensional scan cone. The 3D scan cone provides images of the ultrasound-probed ROI in the form of a rotational 2D scan plane array and is referred to as a V-mode® image or images. The V-mode® images may also be include wedge and translational arrays.

After the scan, the transceiver displays the volume of urine retained within the bladder along with aiming information for the transceiver to enable the correct placement of the probe with respect to the bladder. The aiming information allows the user to repeat the scan as needed to get a well-centered image and/or a complete image of the bladder.

Once the scan is complete, the three-dimensional data may be transmitted securely to a server computer on a remote computer that is coupled to a network, such as the Internet. Alternately, a local computer network, or an independent standalone personal computer may also be used. In any case, image processing algorithms on the computer analyze pixels within a 2D portion of a 3D image or the voxels of the 3D image. The image processing algorithms then define which pixels or voxels occupy or otherwise constitute an inner or outer wall layer. Thereafter, wall areas of the inner and outer layers, and thickness between them, is determined. Organ wall or bladder wall weight is determined as a product of wall layer area, thickness between the wall layers, and density of the wall.

The image processing algorithms delineate the outer and inner walls of the anterior portion of bladder wall within the bladder region and determine the actual surface area, S, of the bladder wall using, for example, a modification of the Marching Cubes algorithm, as utilized from the VTK Library maintained by Kitware, Inc. (Clifton Park, N.Y., USA), incorporated by reference herein. The bladder wall thickness, t, is then calculated as the distance between the outer and the inner surfaces of bladder wall. Finally, as shown in equation E1, the bladder weight is estimated as the product of the surface area, thickness and bladder muscle specific gravity, ρ:

UEBW=S×t×ρ. E1

One benefit of the embodiments of the present invention is that it produces more accurate and consistent estimates of UEBW. The reasons for higher accuracy and consistency include:

The use of three-dimensional data instead of two-dimensional data to calculate the surface area and thickness. In another embodiment, the outer anterior wall of the bladder is delineated to enable the calculation of the bladder wall thickness (BWT);

The use of the measured surface area instead of using surface area based upon a spherical model; and

The automatic and consistent measurement of the bladder wall thickness.

Additional benefits conferred by the embodiments also include its non-invasiveness and its ease of use in that UEBW is measured over a range of bladder volumes, thereby eliminating the need to catheterize the patient to fill up to a fixed volume.

FIGS. 1A-D depicts a partial schematic and partial isometric view of a transceiver, a scan cone array of scan planes, and a scan plane of the array.

FIG. 1A depicts a transceiver 10A having an ultrasound transducer housing 18 and a transceiver dome 20 from which ultrasound energy emanates to probe a patient or subject. Information from ultrasound echoes returning from the probing ultrasound is presented on the display 14. The information may be alphanumeric, pictorial, and describe positional locations of a targeted organ or ROI.

FIG. 1B is a graphical representation of a plurality of scan planes 42 that contain the probing ultrasound. The plurality of scan planes 42 defines a scan cone 40 in the form of a three-dimensional (3D) array having a substantially conical shape that projects outwardly from the dome 20 of the transceivers 10A.

The plurality of scan planes 42 are oriented about an axis 11 extending through the transceivers 10A. One or more, or alternately each of the scan planes 42 are positioned about the axis 11, which may be positioned at a predetermined angular position θ. The scan planes 42 are mutually spaced apart by angles θ₁ and θ₂ whose angular value may vary. That is, although the angles θ₁ and θ₂ to θₙ are depicted as approximately equal, the θ angles may have different values. Other scan cone configurations are possible. For example, a wedge-shaped scan cone, or other similar shapes may be generated by the transceiver 10A.

FIG. 1C is a graphical representation of a scan plane 42. The scan plane 42 includes the peripheral scan lines 44 and 46, and an internal scan line 48 having a length r that extends outwardly from the transceivers 10A and between the scan lines 44 and 46. Thus, a selected point along the peripheral scan lines 44 and 46 and the internal scan line 48 may be defined with reference to the distance r and angular coordinate values φ and θ. The length r preferably extends to approximately 18 to 20 centimeters (cm), although other lengths are possible. Particular embodiments include approximately seventy-seven scan lines 48 that extend outwardly from the dome 20, although any number of scan lines may be used.

FIG. 1D a graphical representation of a plurality of scan lines 48 emanating from the ultrasound transceiver forming a single scan plane 42 extending through a cross-section of portions of an internal bodily organ. The scan plane 42 is fan-shaped, bounded by peripheral scan lines 44 and 46, and has a semi-circular dome cutout 41. The number and location of the internal scan lines emanating from the transceivers 10A within a given scan plane 42 may be distributed at different positional coordinates about the axis line 11 as required to sufficiently visualize structures or images within the scan plane 42. As shown, four portions of an off-centered region-of-interest (ROI) are exhibited as irregular regions 49 of the internal organ. Three portions are viewable within the scan plane 42 in totality, and one is truncated by the peripheral scan line 44.

As described above, the angular movement of the transducer may be mechanically effected and/or it may be electronically or otherwise generated. In either case, the number of lines 48 and the length of the lines may vary, so that the tilt angle φ (FIG. 1C) sweeps through angles approximately between −60° and +60° for a total arc of approximately 120°. In one particular embodiment, the transceiver 10A is configured to generate approximately about seventy-seven scan lines between the first limiting scan line 44 and a second limiting scan line 46. In another particular embodiment, each of the scan lines has a length of approximately about 18 to 20 centimeters (cm). The angular separation between adjacent scan lines 48 (FIG. 1B) may be uniform or non-uniform. For example, and in another particular embodiment, the angular separation φ₁ and φ₂ to φₙ (as shown in FIG. 1B) may be about 1.5°. Alternately, and in another particular embodiment, the angular separation φ₁, φ₂, φₙ may be a sequence wherein adjacent angles are ordered to include angles of 1.5°, 6.8°, 15.5°, 7.2°, and so on, where a 1.5° separation is between a first scan line and a second scan line, a 6.8° separation is between the second scan line and a third scan line, a 15.5° separation is between the third scan line and a fourth scan line, a 7.2° separation is between the fourth scan line and a fifth scan line, and so on. The angular separation between adjacent scan lines may also be a combination of uniform and non-uniform angular spacings, for example, a sequence of angles may be ordered to include 1.5°, 1.5°, 1.5°, 7.2°, 14.3°, 20.2°, 8.0°, 8.0°, 8.0°, 4.3°, 7.8°, and so on.

FIG. 2 depicts a partial schematic and partial isometric and side view of a transceiver 10B, and a scan cone array 30 comprised of 3D-distributed scan lines. Each of the scan lines have a length r that projects outwardly from the transceiver 10B. As illustrated the transceiver 10B emits 3D-distributed scan lines within the scan cone 30 that are one-dimensional ultrasound A-lines. Taken as an aggregate, these 3D-distributed A-lines define the conical shape of the scan cone 30. The ultrasound scan cone 30 extends outwardly from the dome 20 of the transceiver 10B and centered about the axis line 11 (FIG. 1B). The 3D-distributed scan lines of the scan cone 30 include a plurality of internal and peripheral scan lines that are distributed within a volume defined by a perimeter of the scan cone 30. Accordingly, the peripheral scan lines 31A-31F define an outer surface of the scan cone 30, while the internal scan lines 34A-34C are distributed between the respective peripheral scan lines 31A-31F. Scan line 34B is generally collinear with the axis 11, and the scan cone 30 is generally and coaxially centered on the axis line 11.

The locations of the internal and peripheral scan lines may be further defined by an angular spacing from the center scan line 34B and between internal and peripheral scan lines. The angular spacing between scan line 34B and peripheral or internal scan lines are designated by angle Φ and angular spacings between internal or peripheral scan lines are designated by angle Ø. The angles Φ1, Φ2, and Φ3 respectively define the angular spacings from scan line 34B to scan lines 34A, 34C, and 31D. Similarly, angles Ø1, Ø2, and Ø3 respectively define the angular spacing between scan line 31B and 31C, 31C and 34A, and 31D and 31E.

With continued reference to FIG. 2, the plurality of peripheral scan lines 31A-E and the plurality of internal scan lines 34A-D are three dimensionally distributed A-lines (scan lines) that are not necessarily confined within a scan plane, but instead may sweep throughout the internal regions and along the periphery of the scan cone 30. Thus, a given point within the scan cone 30 may be identified by the coordinates r, Φ, and Ø whose values generally vary. The number and location of the internal scan lines 34A-D emanating from the transceiver 10B may thus be distributed within the scan cone 30 at different positional coordinates as required to sufficiently visualize structures or images within a region of interest (ROI) in a patient. The angular movement of the ultrasound transducer within the transceiver 10B may be mechanically effected, and/or it may be electronically generated. In any case, the number of lines and the length of the lines may be uniform or otherwise vary, so that angle Φ may sweep through angles approximately between −60° between scan line 34B and 31A, and +60° between scan line 34B and 31B. Thus, the angle Φ may include a total arc of approximately 120°. In one embodiment, the transceiver 10B is configured to generate a plurality of 3D-distributed scan lines within the scan cone 30 having a length r of approximately 18 to 20 centimeters (cm).

FIG. 3 depicts the transceiver 10A (FIG. 1) removably positioned in a communications cradle 50A that is operable to communicate the data wirelessly uploaded to the computer or other microprocessor device (not shown). The data is uploaded securely to the computer or to a server via the computer where it is processed by a bladder weight estimation algorithm that will be described in greater detail below. The transceiver 10B may be similarly housed in the cradle 50A. In this wireless embodiment, the cradle 50A has circuitry that receives and converts the informational content of the scan cone 40 or scan cone 30 to a wireless signal 50A-2.

FIG. 4 depicts the transceiver 10A removably positioned in a communications cradle 50B where the data is uploaded by an electrical connection 50B-2 to the computer or other microprocessor device (not shown). The data is uploaded securely to the computer or to a server via the computer where it is processed by the bladder weight estimation algorithm. The transceiver 10B may be similarly removably positioned in the cradle 50B. In this embodiment, the cradle 50B has circuitry that receives and converts the informational content of the scan cone 40 or the scan cone 30 to a non-wireless signal that is conveyed in conduit 50B-2 capable of transmitting electrical, light, or sound-based signals. A particular electrical embodiment of conduit 50B-2 may include a universal serial bus (USB) in signal communication with a microprocessor-based device.

FIG. 5 depicts images showing the abdominal area of a patient 68 being scanned by a transceiver 10C and the data being wirelessly uploaded to a personal computer during initial targeting of a region of interest (ROI) that is left of the umbilicus 68 and umbilicus midline 68C. FIG. 5 depicts images showing the patient 68 being scanned by a bladder wall mass system 60A during an initial targeting phase using the transceiver 10C capable of generating wireless signal. The transceiver 10C has circuitry that converts the informational content of the scan cone 40 or scan cone 30 to wireless signal 25C-1 that may be in the form of visible light, invisible light (such as infrared light) or sound-based signals. As depicted, the data is wirelessly uploaded to the personal computer 52 during initial targeting of an organ or ROI. In a particular embodiment of the transceiver 10C, a focused 3.7 MHz single element transducer is used that is steered mechanically to acquire a 120-degree scan cone 42. On a display screen 54 coupled to the computer 52, a scan cone image 40A displays an off-centered view of the organ 56A that is truncated.

The scan protocol for obtaining a UEBW begins by placing the transceiver 10C approximately one inch above the symphysis pubis with the scanhead aimed slightly towards the coccyx. The three-dimensional ultrasound data is collected upon pressing the scan button on the scanner. After the scan is complete, the display 14 on the device 10C displays aiming information in the form of arrows. A flashing arrow indicates to the user to point the device in the arrow's direction and rescan. The scan is repeated until the device displays only a solid arrow or no arrow. The display 16 on the device may also display the calculated bladder volume. The aforementioned aiming process is more fully described in U.S. Pat. No. 6,884,217 to McMorrow et al., which is incorporated by reference as if fully disclosed herein. For the UEBW measurement, the required bladder volume is between 200 and 400 ml. If the bladder volume reading is less than 200 ml, the patient could be given some fluids and scanned after a short time interval. Once the scanning is complete and the patient has bladder volume between 200 and 400 ml, the device may be placed on a communication cradle that is attached to a personal computer. Other methods and systems described below incorporate by reference U.S. Pat. Nos. 4,926,871; 5,235,985; 6,569,097; 6,110,111; and 6,676,605 as if fully disclosed herein.

Expanding on the protocol described above, and still referring to FIG. 5 the system 60A also includes a personal computing device 52 that is configured to wirelessly exchange information with the transceiver 10C, although other means of information exchange may be employed when the transceiver 10C is used. In operation, the transceiver 10C is applied to a side abdominal region of a patient 68. The transceiver 10B is placed off-center from a centerline 68C of the patient 68 to obtain, for example a trans-abdominal image of a uterine organ in a female patient. The transceiver 10B may contact the patient 68 through a pad 67 that includes an acoustic coupling gel that is placed on the patient 68 substantially left of the umbilicus 68A and centerline 68C. Alternatively, an acoustic coupling gel may be applied to the skin of the patient 68. The pad 67 advantageously minimizes ultrasound attenuation between the patient 68 and the transceiver 10B by maximizing sound conduction from the transceiver 10B into the patient 68. As shown in FIGS. 6 and 7 below, an ultrasound imaging system 60B includes a transceiver 10D that is in wired communication with the computer 52. In this wired embodiment, the transceiver 10D has circuitry that receives and converts the informational content of the scan cone 40 or scan cone 30 to a non-wireless signal that is conveyed in the conduit between the transceiver 10D and computer 52, which may include an electrical, a light, or a sound-based signal.

Wireless signals 25C-1 include echo information that is conveyed to and processed by the image processing algorithm in the personal computer device 52. A scan cone 40 (FIG. 1B) displays an internal organ as partial image 56A on a computer display 54. The image 56A is significantly truncated and off-centered relative to a middle portion of the scan cone 40A due to the positioning of the transceiver 10B.

As shown in FIG. 5, the trans-abdominally acquired image is initially obtained during a targeting phase of the imaging. During the initial targeting, a first freehand position may reveal an organ or ROI 56A that is substantially off-center. The transceiver 10C is operated in a two-dimensional continuous acquisition mode. In the two-dimensional continuous mode, data is continuously acquired and presented as a scan plane image as previously shown and described. The data thus acquired may be viewed on a display device, such as the display 54, coupled to the transceiver 10B while an operator physically translates the transceiver 10C across the abdominal region of the patient. When it is desired to acquire data, the operator may acquire data by depressing the trigger 14 of the transceiver 10C to acquire real-time imaging that is presented to the operator on the transceiver display 14. If the initial location of the transceiver is significantly off-center, as in the case of the freehand first position, results in only a portion of the organ or ROI 56A being visible in the scan plane 40A.

FIG. 6 depicts images showing the patient 68 being scanned by the transceiver 10C and the data being wirelessly uploaded to a personal computer of a properly targeted ROI in the abdominal area beneath the umbilicus 68A and near umbilicus midline 68C. Here the patient being scanned by the transceiver 10C and the data is wirelessly uploaded to the personal computer 52 occurs when the ROI 56B is properly targeted. The isometric view presents the ultrasound imaging system 60A applied to a center abdominal region of a patient. The transceiver 10C may be translated or moved to a freehand second position that is beneath the umbilicus 68A on the centerline 68C of the patient 68. Wireless signals 25C-2 having information from the transceiver 10C are communicated to the personal computer device 52. An inertial reference unit positioned within the transceiver 10C senses positional changes for the transceiver 10C relative to a reference coordinate system. Information from the inertial reference unit, as described in greater detail below, permits updated real-time scan cone image acquisition, so that a scan cone 40B having a complete image of the organ 56B can be obtained. Still other embodiments are within the scope of the present invention. For example, the transceiver 10C may also be used in the system 60A, as shown in FIG. 10. The transceiver 10A and the support cradle 50A shown in FIG. 3 as well as the transceiver 10A and the support cradle 50B of FIG. 4 may also be used, as shown in FIG. 7 and FIG. 8 below, respectively. Furthermore, the transceivers 10A or 10B may be equipped with an inertial reference system to determine the location coordinates of the transceivers 10A or 10B in relation to the patient 68. The inertial reference systems may employ accelerometers 22 to determine the translational component of the location coordinates and gyroscopes 23 to determine the rotational component of the location coordinates.

FIG. 7 is a schematic illustration and partial isometric view of a network connected ultrasound system 100 in communication with ultrasound imaging systems 60A-D. The system 100 includes one or more personal computer devices 52 that are coupled to a server 56 by a communications system 55. The devices 52 are, in turn, coupled to one or more ultrasound transceivers, for examples the systems 60A-60D. The server 56 may be operable to provide additional processing of ultrasound information, or it may be coupled to still other servers (not shown in FIG. 7) and devices, for examples transceivers 10A or 10B equipped with snap on collars having an inertial reference system that may employ at least one accelerometer 22 to determine the translational component of the location coordinates and at least one gyroscope 23 to determine the rotational component of the location coordinates.

FIG. 8 is a schematic illustration and partial isometric view of an Internet connected ultrasound system 110 in communication with ultrasound imaging systems 60A-D. The Internet system 110 is coupled or otherwise in communication with the systems 60A-60D through an array of computers 114 in remote signal communication with the server 56. The array of computers 114 may include other computers similar to the computer 52 of systems 60A-D. The system 110 may also be in communication with the transceiver 10A or 10B having inertial reference capability as described above.

FIG. 9A is a B-mode or two-dimensional ultrasound image of a bladder in a transverse section using one of the transceivers 10A-B of FIG. 1A and FIG. 2, respectively, with 3.7 MHz pulse frequency from imaging systems 60A-D. FIG. 9A shows the ultrasound appearance of a transverse section of a bladder lumen 150 visualized as a dark semi-circular or pumpkin-shaped region within scan plane 142. The bladder lumen 150 presents as a dark region in the center region of scan plane 142 due to the nature of fluids or empty spaces within the bladder lumen being hypoechoic. More solid-like tissue barriers are echoic to incoming or probing ultrasound energy and reflect back the incoming probing ultrasound. The barrier-reflected ultrasound present as bright regions with in the scan cone 142. The echoic barriers outlining the bladder perimeter proximal to the transducer dome 41 cutout is shown as a sub-mucosal 146 layer and sub-serosal layer 148 of the anterior region of the bladder wall.

FIG. 9B is a close-up of the image in FIG. 9A showing the anterior bladder wall. The zoomed image results from a series of log-compressed A-mode lines and closely shows the cross-sectional structure of the anterior wall of the bladder 150. A relatively lighter region 147 is shown interposed between the brighter sub-mucosal 146 and sub-serosal layers 146 and 148. The detrusor bladder wall muscle occupies the less bright region 147. Though less bright than layers 146 and 148, the detrusor muscle region 147 is brighter than the bladder lumen 150.

FIG. 9C is a log-compressed A-mode line of one scan line similar to scan line 48 through the bladder and illustrates the relative echogenic as a function of scan line position or depth through the bladder. The sub-mucosal layer of the wall, the sub-serosal layer, and the detrusor muscle are visualized in this particular set of zoomed B-mode and the A-mode data. These two layers of the bladder wall are most clearly visible when the ultrasound beam is normally incident to the bladder wall. As the ultrasound incidence deviates from normal, the two layers start appearing as one and may not be reliability detected. While in many data sets these two layers of the bladder walls are clearly visible at normal incidence, there are some cases when the perivesical tissue (such as the peritoneum) impinges on the bladder wall and merges with the subserosal layer. Some samples of such images where the peritoneum merges with the subserosal layer are shown in FIG. 9C.

As shown in FIG. 9C, a significantly resolving histogram is obtained when an ROI is probed with incoming ultrasound that is substantially normal to the organ or ROI. Here cross-sections of structures of FIGS. 9A and 9B are seen with enough fine detail to be distinguished from each other. A one-dimensional histogram plot of ultrasound echo intensity along an A-line scan line similar to scan line 49 of FIG. 1D, scan lines 31A-F, or scan lines 34A-C of FIG. 2 is plotted against the depth of the scan line. The sub-serosal layer 148 is shown being slightly more echogenic than the more distal sub-mucosal layer 146. The sub-serosal layer 148 is approximately 3 cm from the scan head dome 20 and the sub-serosal layer approximately 3.5 cm from the dome 20. The bladder lumen 150 is shown spanning between the anterior located sub-serosal layer 146 to a more posterior depth near 9.5 cm. Compared to the darker bladder lumen 150, the relatively brighter detrusor region 147 is shown interposed between the even brighter sub-mucosal and sub-serosal layers 146 and 148.

FIG. 10 is an algorithm 170 for the calculation of UEBW from V-mode® ultrasound data. The algorithm permits the calculation of UEBW from V-mode® ultrasound data. Bock 178 in the algorithm 178 is to delineate the bladder region. This delineated bladder region is then used to calculate the bladder surface area as shown at block 186. Using the delineated bladder region and the input V-mode® data obtained at block 172, the anterior wall of the bladder is determined. This anterior wall delineation is used to calculate the bladder wall thickness. Finally, the surface area and the thickness measurements are combined to calculate the UEBW, as shown at block 198. Though algorithm 170 is directed to determining wall weight and/or masses of bladders, algorithm 170 may also be directed to non-bladder regions-of-interests, such as a uterus, a heart, a kidney, or tumors of cancerous and/or non-cancerous origins. Non-cancerous tumors may include parasitic infections having a sack like growth.

FIG. 11 is an expansion of sub-algorithm 172 of FIG. 10. At block 172-2 the ultrasound probe is positioned over an abdomen to ultrasound scan at least a portion of a bladder. Returning echoes from the bladder are received by the transceiver 10A or 10B. Thereafter, at process block 172-6, signals are generated in proportion to the strength of the returning ultrasound echoes. The signals are processed into ultrasound images by image processing algorithms discussed below that are executable by microprocessors located in the computer 52, local server 56, or other servers and computers accessible by the Internet 114. In either case, an image of the bladder is presented for viewing by a user on the computer display 54. Thereafter, at decision diamond 172-8 presents a query “Is bladder sufficiently targeted?”. Methods for determining targeting sufficiency is more fully described in U.S. Pat. No. 6,884,217 to McMorrow et al. which is incorporated by reference herein. If the answer is “no”, sub-algorithm 172 returns to block 172-2 and proceeds to toward decision diamond 172-8. If the answer is “yes”, then sub-algorithm 172 is finished and exits to sub-algorithms 178 and 182 that are subsequently engaged.

FIG. 12A is an expansion of sub-algorithm 178 of FIG. 10. Sub-algorithm 178 begins with entry into decision diamond 178-4 to answer to the query “Is the urine volume between 200 and 400 ml?” expressed in mathematical terms as “200<urine volume<400 ml?”. If the answer is Yes, then Data is processed at block 178-8 to delineate the bladder. If the answer is No, then at block 178-6 the bladder is allowed to accumulate enough urine to be within 200 and 400 ml. After the urine volume falls within 200 and 400 ml, the anterior submucosal and anterior subserosal wall locations are determined at block 178-12. Thereafter, sub-algorithm 178 exits to process block 186 or 192.

FIG. 12B is an expansion of an alternate embodiment of sub-algorithm 178 of FIG. 10. UBEW may be determined at volumes less than 200 ml or greater than 400 ml, though the accuracy may not be optimal compared bladders having between 200 and 400 ml urine. When circumstances do not allow for 200-400 ml to be collected in the bladder, then process block 172 may proceed directly to process block 178-8 wherein the acquired data is processed to delineate the bladder. Thereafter, at process block 178-12, the anterior sub-mucosal and sub-serosal wall locations are determined. From here, the method continues to process block 186 and 192.

FIG. 13 is an expansion of the process data to delineate bladder sub-algorithm 178-8 of FIGS. 12A and 12B. The sub-algorithm 178-8 is comprised of eight process or decision routines and begins after completion of sub-algorithm 172 with the first process block 178-8A referred to as Find Initial Wall. From Find Initial Wall block 178-8A is the next block 178-8B that entitled Find Centroid. Thereafter, block 178-8C is Fixed Initial Walls. After Fix Initial Walls is a decision block 178-8D with a query asking, “Is it a non-bladder?” If the answer is “yes” that the organ is a non-bladder, the next process is Clear Walls block 178-8E. Thereafter, the volume is displayed at block 178-8H and the process 178-8 continues on to sub-process 178-8J. Referring back to decision diamond 178-8D, if the organ is not a non-bladder, that is “no”, then another decision 178-8F presents the query “Is volume less than 40 ml?” If the answer is “no” to the decision diamond 178-8F, then the volume is displayed at terminator 178-8H and the algorithm 178-8 proceeds to sub-algorithm 178-8J. If at decision diamond 178-8F the answer is “yes” to the query, “Is volume less than 40 ml?”, another decision is presented at diamond 178-8G with the query “Is it a bladder region?” If the answer is “no” then the sub-algorithm 178-8 proceeds to the Clear Walls of block 178-8E and thence to terminator block 178-8H Volume Displayed. If at the decision diamond 178-8G, the answer is “yes” to the query, “Is it a bladder region?” then the volume is displayed at terminator 178-8H and process 178-8 continues on to algorithms 180 and 186. In sub-algorithm 178-8, an interface line is overlaid on the B-mode scan plane image to approximate an initial location for an organ wall, for example, a uterus or a bladder. This initial interface line is used as a seed or initial reference point which is further used as a basis to adjust the determination for the inner and outer wall layers of the organ wall. Furthermore, in this algorithm, the detected region in the scan plane is determined to be or not to be a bladder or a uterus. This occurs specifically when a gender button (not shown) of the transceiver 10A (FIG. 1A) indicates that the scan is for a female. If the regions indeed are found to be a uterus, it is cleared and a zero volume is displayed. For a non-uterus region, such as a bladder, if the volume is very small, then checks are made on the size of a signal characteristic inside the detected region to ensure that it is a bladder and not another tissue. If a region is indeed a bladder region it is computed and displayed on the output.

FIG. 14 is an expansion of the Find Initial Walls sub-algorithm 178-8A of FIG. 13. The sub-algorithm 178-8A is comprised of 11 processes loops, decisions, and terminators. Sub-algorithm 178-8A begins with process 180A2 in which the Local Average is calculated for the 15 to 16 samples that functions as a low pass filter (LPF) to reduce noise in the signal. Other embodiments allow for calculating averages from less than 15 and more than 16 samples. Next is block 180A4 in which the gradient is calculated using a central difference formulation and has taken over seven sample sets. The process at block 180A4 then proceeds to a beginning loop limit 180A6. In block 180A6, each sample is examined in a detection region. Thereafter, at decision diamond 180A8, the query is, “Is gradient minimum?” If the answer is “no” then, another query is presented at decision diamond 180A18, the query being, “Looking for BW and gradient maximum?” BW refers to for back wall. If the answer to the query in block 180A18 is “no” then, the end of the loop limit is proceeded to at block 180A30. Thereafter, from the end of the loop limit at 180A30, the terminator end find initial walls is reached at block 180A40. Returning now to the decision diamond 180A8, if the answer to the query, “Is gradient minimum?” “yes” then another query is presented in decision diamond 180A10. The query in 180A10 is “Is candidate FW/BW best?” FW is refers to front wall and BW refers to back wall. If the answer to the query in block 180A10 is “no”, then the process 180A62 is used in which the front wall data is saved and another back wall is looked for. If the query to in 180A10 is “yes” then the process is Save Candidate occurs at block 180A14. Thereafter, the process returns to beginning loop 180A6 to resume. Returning to the decision diamond 180A10, should the answer be “yes” to the query, “Is candidate FW/BW best, then the process proceeds to block 180A12 in which the candidate is assigned as a pair for back wall/front wall.” Thereafter from block 180A12, the algorithm 186A returns to the beginning loop 180A6 and then the process will then terminate at end of each sample at end loop 180A30 and thence to terminator 180A40 for end find initial walls sub-algorithm and proceed to sub-algorithm 178-8B. Sub-algorithm 178-8A attempts to find the best front wall and back wall pair for the inner and outer wall layer plotting points. The best front wall and back wall pair in each scan line is defined as the front wall and back wall pair for which the difference in the back wall gradient and front wall gradient sometimes referred to as the tissue delta, is the maximum and the smallest local average between the front wall and back wall pair is the minimum for the pixel values.

FIG. 15 is an expansion of the Fix Initial Walls sub-algorithm 178-8C of FIG. 13. Sub-algorithm 178-8C is comprised of several processes decision diamonds and loops. Sub-algorithm 178-8C operates on a scan plane by scan plane basis where the first scan plane to be processed is one that is closest to the central aid of the initial walls and then the remaining scan planes are processed moving in either direction of that initial scan plane. Sub-algorithm 178-8C begins at block 180C2 referred to as Start Fix Initial Walls. The first process is at block 180C4 in which the center line is corrected if necessary. The center line is defined as the line on that scan plane with the maximum gradient difference between the front wall and the back wall. The correction of the front wall and the back wall location at any line is carried out by a match filtering like step where the best location within a search limit is defined as the one for which the difference between points immediately outside the bladder and points immediately inside the bladder is maximum. Of course, this applies to any organ other than the bladder, as the bladder is used here as an example of a particular embodiment. Thereafter, at block 180C6, the front wall and back wall means are calculated for five central lines. The pixel main intensity is computed and if this intensity is less than expected from the noise at that depth, the lines are cleared and the algorithm proceeds to the next plane as shown in decision diamond 180C8 to the query, “Is BW level less than noise?” where BW means the back wall (or posterior wall) of the bladder. If the answer is “yes” to this query, at block 180C10, the process Clear Wall Data is initiated and from that proceeds to terminator 180C50 End Fix Initial Walls. Returning to the decision diamond 180C8, if the answer is “no” to the query, “Is BW level less than noise?” then the sub-algorithm 180C proceeds to the process at block 180C12 described as Fix 3 Central Lines. From this point through the end of sub-algorithm 180C, the purpose is first correct the lines to the left of the central lines, called the left half plane (LHP) until either the edge of the bladder or the edge of the ultrasound cone is found. After the algorithm corrects the LHP, it proceeds to correct the lines to the right of the central lines, called the right half plane. Because the same steps are used for all lines, regardless of their position to the left of center or to the right of center, the process blocks 180C16 through 180C42 are used for both the LHP and once for the right half plane. The “line index” of process 180C14 indicates an identifier for the current line that is processed. The line index is set to 2 indices less than the center line to start processing the LHP. The looping procedure started in block 180C16 continues looping while the line index is a valid index (i.e. it corresponds to a scan line). Sub-loop 180C18 is started with the intent of adjusting the initial wall locations, sub-process 180C20, to their correct location if any correction is necessary. This loop, terminated at process 180C24, completes two iterations. The first iteration uses sub-process 180C20 to correct the front wall of the bladder on the current line and the second iteration to correct the back wall of the bladder, although the ordering of which wall is corrected first can be interchanged. Once the wall locations have been corrected of the current line have been corrected, sub-algorithm 180C proceeds to sub-process 180C28, “Check Wall Growth”. This sub-process ensures that the length of the scan line that intersects the bladder in the current line does not grow significantly with respect to the previous line that has already been corrected. In the preferred embodiment, the length of the scan line intersecting the bladder is constrained to be less than 1.125 times longer than in the previous line. If the loop bounded by sub-processes 180C16 and 180C42 is being applied to the LHP, then the previous line is one index number greater than the current line index. Otherwise, the previous line index is one index number less than the current index. After completing sub-process 180C28, the sub-process 180C30 “Check Wall Consistency” verifies that the portion of the current scan line that intersects the bladder overlaps the portion of the previous scan line that intersects the bladder. After completing sub-process 180C30, decision 180C32 queries “If working LHP?” (i.e. the loop bounded by terminators 180C16 and 180C42 is being applied to the lines left of center). If the answer to the query is yes, then the sub-process 180C34 “Decrement line index” decreases the line index by one index number. Decision 180C36 queries “If line index is invalid”. The loop bounded by terminators 180C16 and 180C42 is applied to the next, and now current, scan line. If the decremented line index corresponds to an invalid value, the edge of the LHP has been reached. Sub-process 180C38 is called to reset the line index to the first line to the right of center that has not been adjusted. The loop bounded by terminators 180C16 and 180C42 will now be applied to the right half plane (RHP). Returning to decision 180C32, if the answer to the query is “No”, sub-process 180C40 “Increment line index” results with the line index being increased by one index number. Loop terminator 180C42 cause the loop to return to 180C16 as long as the line index corresponds to an actual scan line. As soon as that condition is violated, the loop terminator will cause sub-algorithm 178-8C to proceed to the terminator 180C50, “End Fix Initial Walls” and proceed to sub-algorithm 178-8D.

FIG. 16 is an expansion of surface area sub-algorithm 186 of FIG. 10. Once the bladder is delineated at sub-algorithm 178, the process continues to block 186-2 wherein the 2-D scan planes are assembled into a 3-D scan cone such as the scan cone 40 of FIG. 1B or scan cone 30 of FIG. 2 and the sub-serosal layer within the 3-D array. When the 3-D scan cone is comprised of scan planes substantially similar to scan plane 42 where the scan lines 48 are confined within a given scan plane 42, the 3-D array may include scan planes assembled into a rotational array, a wedge array, and a translational array. Alternatively, the, the 3-D scan cone, in the form of the scan cone 30 of FIG. 2, is made of a randomly distributed assembly of 3-D distributed scan lines that are not confined to be within any given scan plane. Then, at process block 186-6, the sub-serosal layer is partitioned into a triangular assembly. Thereafter, the surface area of the sub-serosal layer is calculated using a Marching Cubes or other appropriate algorithm at process block 186-10. Knowing the surface area now permits calculation of organ or bladder mass in view of thickness determinations, and organ wall density as described below.

FIG. 17 is an expansion of the calculate surface area sub-algorithm 186 of FIG. 10 for sub-mucosal layer 146. Once the bladder is delineated at sub-algorithm 178 and the sub-serosal layer 148 location approximated, the process 186 begins with block 186-12 wherein the 2-D scan planes are assembled into a 3-D scan cone similar to scan cone 40 or scan cone 30 and the sub-mucosal layer within the 3-D distributed scan lines that are not confined to be within any given scan plane. Then, at process block 186-16, the sub-mucosal layer is partitioned into a triangular assembly. Thereafter, the surface area of the sub-mucosal layer is calculated using a Marching Cubes or other appropriate algorithm at process block 186-20. Knowing the surface area now permits calculation of organ or bladder mass in view of thickness determinations, and organ wall density as described below.

FIG. 18 is an expansion of the sub-algorithm 186-10 of FIG. 16 for sub-serosal layer 148. The algorithm 186-10 begins with block 186-10A by creating a triangular mesh of voxels or pixel volume elements defined as an iso-surface or a provisional working representation of the sub-serosal layer 148. Thereafter, at block 186-10C, the iso-surface layer is defined as a plurality of voxel cubes having eight pixel vertices. Thereafter, at block 186-10E, the vertices of the pixel values are compared with a selected threshold voxel or pixel intensity value for the purposes of sorting or classifying voxels as being part of the iso-surface or provisional working representation of the sub-serosal layer 148. How the voxels are sorted is described by block 186-10G. The voxels are defined as being a member or non-member of the iso-surface of the sub-serosal layer 148. A voxel member is defined to be a member if the voxel member has a brightness intensity greater than the threshold value, and a non-member if the voxel has a brightness intensity equal to or less than the threshold value. After sorting and classifying voxels, at block 186-10J, a vertex index value is defined and normals are constructed to the voxels. Thereafter, at block 186-10M, the voxels that are members of the iso-surface are calculated and summed to obtain an accumulated surface area for the sub-serosal layer 148. The algorithm then continues to sub-algorithm 198.

FIG. 19 is an expansion of the sub-algorithm 186-20 of FIG. 16 for sub-mucosal layer 146. The algorithm 186-20 begins with block 186-20A by creating a triangular mesh of voxels or pixel volume elements defined as an iso-surface or a provisional working representation of the sub-serosal layer 148. Thereafter, at block 186-20C, the iso-surface layer is defined as a plurality of voxel cubes having eight pixel vertices. Thereafter, at block 186-20E, the vertice pixel values are compared with a selected threshold voxel or pixel intensity value for the purposes of sorting or classifying voxels as being part of the iso-surface or provisional working representation of the sub-serosal layer 148. How the voxels are sorted is described by block 186-20G. The voxels are defined as being a member or non-member of the iso-surface of the sub-serosal layer 148. A voxel member is defined to be a member if the voxel member has a brightness intensity greater than the threshold value, and a non-member if the voxel has a brightness intensity equal to or less than the threshold value. After sorting and classifying voxels, at block 186-20J, a vertex index value is defined and normals are constructed to the voxels. Thereafter, at block 186-20M, the voxels that are members of the iso-surface are calculated and summed to obtain an accumulated surface area for the sub-serosal layer 148. The algorithm then continues to sub-algorithm 198.

FIG. 20 is an expansion of calculate thickness sub-algorithm 192 of FIG. 10. Once the inner (sub-mucosal) and the outer (sub-serosal) layers of the anterior bladder muscle have been delineated, the thickness calculation involves determining the distance between the two surfaces. From block 180, process 192 begins with block 192-2 where the pixels having maximal echo signals are identified to obtain inner and outer wall layer loci. Thereafter, at block 192-6, the thickness of the organ wall is calculated as a difference between the inner and outer wall layer loci pixel locations. The average distance between the inner and outer wall loci are determined on all scan lines approximately normal to the bladder surface. The distance is reported as output and also used for the bladder weight calculation. The rendered bladder wall on the output images shows this average thickness plotted along the two leading edges of the bladder muscle. From here, process algorithm 192 exits to algorithm 198.

FIG. 21 shows sample delineations of the bladder in scan planes 242-2, 4, and 6 respectively. Here the perimeter of the bladder lumen 250-2, 4, and 6 is outlined by sub-mucosal layers 246-2, 4, and 6 using the Find Initial Walls sub-algorithm 180A as previously described. The outlining approximates the general location of the sub-mucosal layers 246-2, 4, and 6 for the purposes of delineating the perimeter of the hypo-echoic bladder lumen 250-2, 4, and 6 to provide a basis to estimate urine volume. The urine volume is estimated to assess whether or not the bladder contains between 200 and 400 ml so that more exacting positioning of the sub-mucosal and sub-serosal layers may be determined by sub-algorithms 178-12, 186, 186-10, and 182-20. Brighter regions are visible anterior to the sub-mucosal layers 246-2, 4, and 6 and towards the dome cutout 41. Regions posterior to sub-mucosal layers 246-2, 4, and 6 are brighter than bladder lumen 250-2, 4, and 6 due to the more echogenic nature of the posterior tissues.

Once the inner surface or of the bladder wall or sub-serosal has been delineated on a set of data planes, the computer graphics algorithm known as the Marching Cubes algorithm, or other appropriate algorithm, may be used to calculate the 3D surface area of the bladder. The Marching Cubes algorithm creates a triangulated three-dimensional surface that is rendered by a computer graphics engine, for example, the VTK Library available from Kitware, Inc., Clifton Park, USA. Pixel intensity values of the triangle vertices dictate whether or not a given pixel constitutes a member of a given wall layer. For example, pixel values below a selected threshold value define a pixel location that is not a pixel member of a surface layer, and pixel values above a threshold value are defined as a surface layer member. Once the triangulated surface is available, calculating the surface area of that 3D surface is achieved by summing up the areas of all the triangles constituting the 3D surface.

Using the delineated bladder surface as a starting point, the anterior wall of the bladder muscle is then determined to enable thickness calculation. For bladder wall finding, the following model is used. When the ultrasound beam is normally incident to the bladder surface, the bladder wall appears as two bright regions representing the sub-mucosal plus mucosal layer and the subserosal layer, separated by a dark region representing the detrusor muscle as shown in FIG. 9C. Thus, first the angle of incidence of a scan line to the bladder surface is determined and then on all scan lines approximately normal to the bladder surface, two bright peaks immediately anterior to the vesicle lumen are located automatically and are labeled as the inner and the outer walls of the bladder muscle.

FIG. 22 are a first set of normal and magnified saggital images visualized by the ultrasound transceivers 10A-B. FIG. 22 illustrates a sample of bladder wall delineations of the anterior bladder adjacent to bladder lumens 250-10, 12, and 14 of the bladder in scan planes 242-10, 12, and 14 respectively. The upper panel of three images is near normal view and shows the full images. The bottom three are magnified or zoomed images of the solid-line highlighted square inset. The sub-mucosal layers 246-10, 12, and 14 and sub-serosal layers 248-10, 12, and 14 of the bladder wall is depicted in dashed lines overlaid in the magnified images. Once the inner (sub-mucosal) and the outer (sub-serosal) layers of the anterior bladder muscle have been delineated, the thickness calculation involves determining the distance between the two surfaces. The average distance between the inner and outer wall are determined on all scan lines approximately normal to the bladder surface—this distance is reported as output and also used for the bladder weight calculation. The rendered bladder wall on the output images shows this average thickness plotted along the two leading edges of the bladder muscle. In cases where the perivesical tissue merges with the subserosal layer of the bladder, the ultrasound reflection from the perivesical tissue merges with the reflection from the subserosal layer with the result that the peak representing the subserosal layer is less well defined and the bladder wall thickness is overestimated.

FIG. 23 are a second set of normal and magnified saggital images visualized by the ultrasound transceivers 10A-B and illustrates a sample of bladder wall delineations of the anterior bladder adjacent to bladder lumens 250-16, 18, and 20 of the bladder in scan planes 242-16, 18, and 20 respectively. The upper panel of three images is near normal view. The bottom three are magnified images of the solid-line highlighted square inset. The sub-mucosal layers 246-16, 18, and 20 and sub-serosal layers 248-16, 18, and 20 of the bladder wall is depicted in dashed lines overlaid in the magnified images that represents the measured thickness of the bladder wall. The positioning of and the separation between the overlaid thickness lines is automatically determined. The saggital images are visualized so that the peritoneum and the subserosal layer of the bladder wall is automatically distinguished from each other. The delineations of the bladder wall show that thickness overestimates may occur when the perivesical tissue, such as the peritoneum, merge with the subserosal layer of the bladder wall.

Once the bladder wall thickness, t, and the surface area, S, are available, UEBW is simply calculated per equation E1:

UEBW=S×t×ρ. E1

The specific gravity, ρ, used for UEBW calculation is 0.957 as measured by Kojima et al.

FIG. 24 is a schematic representation of four surface patch elements. Particular embodiments for the processing of the surface patch elements may be undertaken by different surface processing algorithms. For example the B-spline interpolation algorithms described in U.S. Pat. No. 6,676,605, herein incorporated by reference, or by application of the Marching Cubes algorithm as utilized from the VTK Library maintained by Kitware, Inc. (Clifton Park, N.Y., USA), also incorporated by reference herein. As depicted in three dimensions in FIG. 24, by way of example, five scan planes 320-328 are seen transmitted substantially longitudinally across a subserosal wall location 332 referenced to a tri-axis plotting grid 340. The five scan planes include the first scan plane 320, the second scan plane 322, the third scan plane 324, the fourth scan plane 326, and the fifth scan plane 328. The scan planes are represented in the preceding formulas as subscripted variable j. Substantially normal to the five longitudinal scan planes are five latitudinal integration lines 360-368 that include a first integration line 360, a second integration line 362, a third integration line 364, a fourth integration line 366, and a fifth integration line 368. The integration lines are represented in the preceding formulas as subscripted variable i.

The four surface patch functions are highlighted in FIG. 24 as the subserosal wall location 372. The i and j subscripts mentioned previously correspond to indices for the lines of latitude and longitude of the bladder surface. For the purposes of this discussion, i will correspond to lines of longitude and j will correspond to lines of latitude although it should be noted the meanings of i and j can be interchanged with a mathematically equivalent result. Using the scan plane and integration line definitions provided in FIG. 20, the four surface patch functions are identified, in the clockwise direction starting in the upper left, as s322,362, s324,362, s324,364, and s322,364.

The surface patches are defined as functions of the patch coordinates, sij(u,v). The patch coordinates u and v, are defined such that 0≦u, v<1 where 0 represents the starting latitude or longitude coordinate (the i and j locations), and 1 represents the next latitude or longitude coordinate (the i+1 and j+1 locations). The surface function could also be expressed in Cartesian coordinates where si,j(u,v)=xi,j(u,v)i+yi,j(u,v)j+zi,j(u,v)k where i, j, k, are unit vectors in the x-, y-, and z-directions respectively. In vector form, the definition of a surface patch function as given in Equation 1 above describes k, are unit vectors in the x-, y-, and z-directions respectively as shown in the equation below. In vector form, the definition of a surface patch function is given in equation E2.

$${s}_{i , j} ( u , v ) = [ \begin{matrix}{x}_{i , j} ( u , v ) \\ {y}_{i , j} ( u , v ) \\ {z}_{i , j} ( u , v )\end{matrix} ] \qquad E 2$$

With the definitions of surface patch functions complete, attention can turn to the surface area calculation represented in the fifth block 206-10 of FIG. 20. The surface area of S, A(S), can be defined as the integral of an area element over the surface S, as shown in equation E3.

$$A ( S ) = {\int }_{s} d A \qquad E 3$$

Since S is composed of a number of the patch surface functions, the calculation for the area of the surface S can be approximated as the sum of the areas of the individual surface patch functions as in equation E4.

$$A ( S ) = {\sum }_{i , j} A ( {s}_{i , j} ) . \qquad E 4$$

The area of the surface patch is the integration of an area element over the surface patch, as shown in equation E5.

$$A ( {s}_{i , j} ) = {\int }_{{s}_{i , j}} d {A}_{i , j} \qquad E 5$$

FIG. 25 is a schematic representation of three scan lines passing through the subserosal and submucosal wall locations of an organ, here illustrated for a bladder. Three scan lines 362, 364, and 366 penetrate the bladder. The dotted portion of the lines represents the portion of the scan lines that passes through the bladder muscle wall at an anterior or front wall location 370A and a posterior or back wall location 370B. The first 362, the second 364, and the third 366 scan lines are shown transmitting through the front subserosal wall location 372A and front submucosal wall location 374A. Similarly, the first 362, the second 364, and the third 366 scan lines are shown transmitting across the internal bladder region 375 and through the back submucosal wall location 374B and back subserosal wall location 372B. The front and back subserosal locations 372A and 372B occupy an outer bladder wall perimeter and the front and back submucosal locations 374A and 374B occupy an inner bladder wall perimeter. A bladder wall thickness value 376 is obtained for the respective differences along each scan line 362-366 between the subserosal wall locations 372A and the submucosal wall locations 374A, or the subserosal wall locations 372B and the submucosal wall locations 374B. The maximum, minimum and mean values of these thicknesses are used in the bladder wall mass calculation and historical tracking of data. In a selected embodiment, the bladder is assumed to have a uniform wall thickness, so that a mean wall thickness value is derived from the scanned data and used for the determination of the bladder lumen volume 375. Although three scan lines are shown in a plane, which are separated by 7.5 degrees from each other. Both the number of scan lines in the plane and the angles separating each scan line within a plane may be varied.

Once the bladder wall thickness and the inner and outer surface area have been measured, the volume of an organ internal region such as the bladder lumen 375 may be calculated by the determining the respective differences between the front and back submucosal wall locations 374A and 374B along each scan line penetrating the bladder lumen 375. The difference between the front and back submucosal wall locations 374A and 374B defines an inter-submucosal distance. The internal volume of the bladder lumen 375 is then calculated as a function of the inter-submucosal distances of the penetrating scan lines and the area of the subserosal boundary or internal bladder perimeter. The volume of bladder lumen 375 is assumed to be the surface area times a function of the inter-submucosal distances, where the assumption is further based on a uniform wall subserosal boundary at all points around the internal bladder perimeter. In the embodiment shown, this volume calculation corresponds to the eighth block 206-20 of FIG. 19.

The methods to obtain the wall-thickness data, the mass data, and the volume of bladder lumen 375 via downloaded digital signals can be configured by the microprocessor system for remote operation via the Internet web-based system. The Internet web-based system (“System For Remote Evaluation Of Ultrasound Information Obtained By A Program Application-Specific Data Collection Device”) is disclosed in detail in U.S. Pat. No. 6,569,097 to Gerald McMorrow et al., herein incorporated by reference. The internet web-based system has multiple programs that collect, analyze, and store organ thickness and organ mass determinations. The alternate embodiment thus provides an ability to measure the rate at which internal organs undergo hypertrophy with time and permits disease tracking, disease progression, and provides educational instructions to patients.

FIGS. 26-28 illustrates in tabular and graphic form the UEBW determinations from seventeen healthy male subjects between the ages of 24 and 55. Each subject was scanned during two or three visits within a period of one week. A registered ultrasonographer scanned each subject with three different BVM6500 devices. The sonographer also scanned the subjects with a freehand translatable ultrasound transceiver using a 10-5 Mhz linear array probe. The bladder wall thickness was manually measured on transverse and on saggital images on the freehand translation ultrasound probe from the leading edge of subserosal layer to the leading edge of the submucosal plus mucosal layer. The subject then voided into a uroflow device to measure the total voided volume. Finally, the post-void residual volume (PVR) was measured using the same three BVM6500 devices. All scans which were outside the specified 200 ml to 400 ml volume range were rejected from the analysis. Also, based on aiming arrow information all scans that did not produce well-centered or well-aimed images were also rejected from the analysis. By visual inspection, all cases where the peritoneum merged with the subserosal layer of the bladder were also identified.

FIG. 26 depicts UEBW measurements for a 17 member subject group. Particular embodiments measured the average UEBW on healthy male subjects to be 46 g with a standard deviation of 8.5 g between the various subjects on a total of 103 exams. FIG. 7 shows the actual UEBW measurements for the different subjects. The UEBW was found to be fairly consistent across a single subject at different volumes between 200 ml and 400 ml and between different instruments. The average coefficient of variation (the standard deviation divided by the mean) in the UEBW measurement was 8% and ranged between 2% to 19% for the different subjects. When calculating UEBW by multiplying the thickness measured by the sonographer using the freehand translation ultrasound machine and the surface area measured by the particular embodiments of UEBW device, an average coefficient of variation of 11% was found, indicating a somewhat lower consistency in manual measurement of thickness.

FIG. 27 depicts UEBW measurements for the subject group after excluding cases where the peritoneum merged with the subserosal layer of the bladder wall. Of the 17 subjects, 11 visually identified cases were excluded where the peritoneum merged with the subserosal layer. Of these 11, the average coefficient of variation in the UEBW measurement dropped to 6% with a minimum of 2% and a maximum of 9%. A plot of the remaining 92 UEBW measurements for the 17 subjects is also shown in FIG. 8.

FIG. 28 shows the bladder surface area calculated by particular method embodiments plotted against the bladder volume. The bladder surface area calculated by the methods of the particular embodiments and plotted against the bladder volume. The gray line in the figure shows the bladder surface area if it is assumed the bladder to be a spherical structure. The bladder surface area calculated by the particular embodiments of the method is on an average 18% higher (p value<0.001, minimum of 3% and maximum of 67%) than the surface area calculated under the spherical assumption, indicating that, as expected, the bladder surface cannot be well approximated by a sphere.

The pre-void bladder volume measured by the particular embodiments of the device was compared to the sum of the uroflow measured voided volume and the post-void residual. A mean difference of −4.6% (95% confidence interval, CI, of −2.7% to −6.4%) was found in the volume measurement which corresponds to a difference of −17 ml (95% CI of −11 to −23).

The particular embodiments provide an automatic and convenient method to estimate UEBW. The results show that UEBW can be consistently and accurately determined using 3-D V-mode® ultrasound. The accuracy and reproducibility improve when the 3D ultrasound scan is well centered and the bladder volume is between 200 and 400 ml. Aiming information and bladder volume measurement is provided immediately to the user to acquire the optimal scan.

Although several researchers have previously proposed the measurement of UEBW, their methods have had several limitations that the particular embodiments overcome. The accuracy of existing methods to estimate bladder weight is limited because of the assumption that the bladder is spherical in shape. The particular embodiments provide results that show the bladder to be significantly non-spherical in shape. In addition, since in the existing methods, the thickness is measured manually, the bladder wall measurements suffer from high inter- and intra-observer variability. Moreover, such measurements in everyday practice are difficult due to both the requirement of filling the patient's bladder to a known fixed volume using a catheter and the required availability of an expensive high-resolution B-mode ultrasound machine and an ultrasound technician. The particular embodiments are non-invasive, accurate, reliable and easy to use.

The 8% average coefficient of variability, CV, in UEBW found using the particular embodiments of the method results from a combination of several sources of variability which need to be studied further. Errors in surface area and thickness measurements are two of the possible sources of variability. Differences between the three devices used are another possible source of variability. Yet another source of variability is due to diurnal variations in the actual bladder weight. Yet another possible source of variability is the bladder weight itself, as measured by the particular embodiments of the method, may not be constant at all bladder volumes.

The particular embodiments provides average UEBW measurements for normal subjects to be somewhat higher than the 35 grams average value reported by Kojima et al. This difference may be explained by their assumption of a spherically shaped bladder imposed by Kojima. The actual bladder shape is significantly different from a sphere and using the actual surface area will lead to a UEBW measurement that is at least 18% higher. A second reason for the difference between their UEBW measurements and the particular embodiments may be the method of measuring thickness. The particular embodiments measure wall thickness by measuring the distance between the visible peaks in the sub-mucosal plus mucosal layer and the subserosal layer. Kojima et al. however, measure bladder wall thickness via a leading-to-leading edge distance. This leading-to-leading edge distances contributes to some differences of bladder weight.

The particular embodiments provide for an automatic, convenient, and consistent method to estimate UEBW as a diagnostic marker for bladder outlet obstruction problems. Ultrasound-estimated bladder weight (UEBW) has the potential to become an important indicator for the diagnosis of bladder outlet obstruction (BOO). The various embodiments established an approach to accurately, consistently, conveniently, and non-invasively measure UEBW using three-dimensional ultrasound imaging. A three-dimensional (3D) image of the bladder is acquired using a hand-held ultrasound machine. The infravesical region of the bladder is delineated on this 3D data set to enable the calculation of bladder volume and the bladder surface area. The outer anterior wall of the bladder is delineated to enable the calculation of the bladder wall thickness (BWT). The UEBW is measured as a product of the bladder surface area, the BWT, and the bladder muscle specific gravity. The UEBW was measured on 17 different healthy subjects and each subject was imaged several times at different bladder volumes to evaluate the consistency of the UEBW measurement. Our approach measured the average UEBW on healthy subjects to be 46 g (σ=8.5 g). The UEBW was found to be fairly consistent with an average coefficient of variability of 8% across a single subject at different bladder volumes between 200 ml and 400 ml. Our surface area measurements show that the bladder shape is significantly non-spherical.

Accordingly, the scope of the invention is not limited by the disclosure of these preferred and alternate embodiments. Instead, the invention should be determined entirely by reference to the claims that follow.

What is claimed is:

1. A method to determine bladder wall thickness using an ultrasound transceiver, the method comprising:
positioning an ultrasound transceiver exterior to a patient such that at least a portion of the bladder wall is within the range of the transceiver;
transmitting radio frequency ultrasound pulses to, and receiving those pulses echoed back from, the external and internal surface of the portion of the bladder wall; and, based on those pulses
calculating for the portion of the bladder wall
(a) the surface area of the external and internal surfaces, and
(b) the distance between the external and internal surfaces.

2. The method of claim 1, wherein the radio frequency ultrasound pulses are sent to the bladder in one or more of the forms selected from the group consisting of a scanplane, a spiral, and a random line.

3. The method of claim 2, wherein the form selected is a scanplane, and the scanplane is associated with an array, the array selected from the group consisting of a translational array, a wedge array, and a rotational array.

4. The method of claim 3, wherein the scanplane in the array is selected from the group consisting of uniformly spaced, non-uniformly spaced, and a combination of uniformly spaced and non-uniformly spaced scanplanes.

5. The system of claim 4, wherein the scanplane comprises a plurality of scanlines, the scanlines selected from the group consisting of uniformly space, non-uniformly spaced, and a combination of uniformly space and non-uniformly spaced scanlines.

6. The system of claim 5, wherein the uniform spacing between each scanplane is approximately 7.5 degrees.

7. The system of claim 5, wherein the uniform spacing between each scanline is approximately 1.5 degrees.

8. The method of claim 1, wherein the echoes are classified into latitudinal and longitudinal components.

9. The method of claim 8, wherein the latitudinal and longitudinal components of the echoes reflecting back from the area of the portion of the bladder wall is defined to be S, and comprises a plurality of surface patches, s_{i,j}, where i and j represent the latitude and longitude components, such that the area of S of the portion of the bladder wall is the sum of the plurality of patches, S=Σs_{i,j}.

10. The method of claim 9, wherein the surface patch s_{i,j} is further defined by a vector s_{i,j}(u,v)=x_{i,j}(u,v)i+y_{i,j}(u,v)j+z_{i,j}(u,v)k, where i, j, k, are unit vectors in the x-, y-, and z-directions respectively, and u and v are surface patch coordinates.

11. The method of claim 1, wherein the thickness separating the surface areas is fdr determined from the relationship
$${fd}_{r} = \frac{log ( \frac{max ( {RF}_{r = r - w / 2 , r + w / 2} ) - min ( {RF}_{r = r - w / 2 , r - w / 2} ) + w}{w} )}{log ( \frac{n}{w} )}$$
wherein the terms max (RF_{r=r-w/2, r+w/2}) and min (RF_{r=r-w/2, r+w/2})+w refer to the maximum and minimum radio frequency (RF) value for a window of length w, centered at a given depth, r, along a scanline of a given number of samples, n, such that the fractal dimension is calculated from the difference between the maximum radio frequency (RF) signal value in the window centered at a given depth, r, then normalized with a total number of samples in a scanline, n.

12. The method of claim 11, wherein the thickness separating the inner and outer wall area fdr is adjusted by a parabolic function of the form is determined from the relationship fdᵢ=arᵢ²+brᵢ+c+εᵢ, where there are 3 parameters (a, b, and c) that define a parabola function with the depth along a scanline r, and the addition of a random element ε, wherein the subscript i indicates a specific value of r, fd, and ε.

13. The method of claim 12, wherein the parabolic function is at least 97% of the maximal value of a fractal dimension is determined from the relationship
$${r}_{97 \%} = \frac{- \hat{b} \pm \sqrt{{\hat{b}}^{2} - 4 \hat{a} ( \hat{c} + 0.97 \frac{{\hat{b}}^{2} + 4 \hat{c}}{4 \hat{a}} )}}{2 \hat{a}} ,$$
where the parameters with hats (̂) indicate that the value is the least-squares estimate of those parameters.

14. The method of claim 1, wherein the area each bladder wall is determined for bladders containing approximately 0 ml to approximately 1000 ml.

15. A method to measure wall thickness of an organ using an ultrasound transceiver, the method comprising:
positioning an ultrasound transceiver exterior to a patient such that at least a portion of an organ wall is within the range of the transceiver;
transmitting radio frequency ultrasound pulses as scanlines to, and receiving those pulses echoed back from, the external and internal surface of the portion of the organ wall, and based on those pulses, forming at least one two-dimensional image;
selecting wall loci at a first position of the organ wall from the two dimensional image;
adjusting the position of the wall loci by applying a one-dimensional analysis of the pulse echoes associated with the two-dimensional image to a second position and a third position; and
determining the thickness of the organ wall by calculating the difference of the wall loci between the second and third positions.

16. The method of claim 15, wherein the radio frequency ultrasound pulses are sent to the organ in one or more of the forms selected from the group consisting of a scanplane, a spiral, and a random scanline.

17. The method of claim 16, wherein the form selected is a scanplane, and the scanplane is associated with an array, the array selected from the group consisting of a translational array, a wedge array, and a rotational array.

18. The method of claim 17, wherein the scanplane in the array is selected from the group consisting of uniformly spaced, non-uniformly spaced, and a combination of uniformly spaced and non-uniformly spaced scanplanes.

19. The method of claim 18, wherein the scanplane comprises a plurality of scanlines, the scanlines selected from the group consisting of uniformly space, non-uniformly spaced, and a combination of uniformly space and non-uniformly spaced scanlines.

20. A method of determining organ wall mass, comprising:
positioning an ultrasound transceiver exterior to a patient such that at least a portion of the organ wall is viewable by the transceiver;
transmitting radio frequency ultrasound pulses and receiving echoic pulses corresponding to the transmitted pulses echoed back from eternal and internal surface portions of the organ wall; and
calculating for the portion of the organ wall at least one of:
a surface area of the external and internal surfaces of the organ wall;
a thickness between the surfaces; and
a mass between the surfaces.

21. The method of claim 20, wherein calculating the organ wall is delineated in a 3-D depiction.

22. The method of claim 21, wherein calculating the surface area is determined by a marching cubes algorithm applied to the 3-D depiction.

23. The method claim 22, wherein determining 3-D depiction includes an array of 2-D scan planes and an array of 3-D distributed scan lines.

24. The method claim 23, wherein positioning the transceiver generates ultrasound pulse echoes that are substantially normal to the organ wall.

25. The method of claim 34, wherein the organ wall is a bladder wall.
